(19) United States
(12) Patent Application Publication
(10) Pub. No.: US 2016/0129155 A1
(43) Pub. Date: May 12, 2016

(54) MUSCULOSKELETAL TISSUE FABRICATION

(71) Applicant: University of Pittsburgh - Of the Commonwealth System of Higher Education, Pittsburgh, PA (US)

(72) Inventors: Hang Lin, Pittsburgh, PA (US); Rocky Sung Chi Tuan, Pittsburgh, PA (US)

(73) Assignee: University of Pittsburgh - Of the Commonwealth System of Higher Education, Pittsburgh, PA (US)

(21) Appl. No.: 14/934,646

(22) Filed: Nov. 6, 2015

Related U.S. Application Data
(60) Provisional application No. 62/077,020, filed on Nov. 7, 2014.

(51) Int. Cl. A61L 27/38; A61L 27/20; A61N 5/06; A61L 27/58; A61L 27/54; A61L 27/22; A61L 27/50

(52) U.S. Cl. CPC A61L 27/3834; A61L 27/3847; A61L 27/222; A61L 27/20; A61L 27/50; A61L 27/58; A61L 27/54; A61L 27/3852; A61N 5/062; A61L 2300/64; A61L 2400/06; A61L 2300/412; A61L 2430/02; A61L 2430/06

(57) ABSTRACT

Described herein are methods of fabricating human cell-based engineered musculoskeletal tissues (hCEMTs) using three dimensional fabrication technology that involves injectable materials with in situ polymerization/solidification capability and/or solid free-form fabrication. Also described is the usage of hCEMTs for tissue repair and drug testing.

### CROSS REFERENCE TO RELATED APPLICATIONS

This application claims the benefit of U.S. Provisional Patent Application No. 62/077,020, filed Nov. 7, 2014, which is incorporated herein by reference in its entirety.

### ACKNOWLEDGMENT OF GOVERNMENT SUPPORT

This invention was made with government support under Grant No. TR000532 awarded by the National Institutes of Health, and Grant Nos. W81XWH-10-1-0850 and W81XWH-08-2-0032 awarded by the ARMY/MRMC. The government has certain rights in the invention.

### FIELD

The present disclosure relates to the fabrication of engineered human musculoskeletal tissues and their application for tissue repair and for drug testing.

### BACKGROUND

In the United States, musculoskeletal disorders, including those affecting bone, cartilage, muscle, and tendon/ligament, result in 70 million office visits and 130 million clinical treatments annually. For example, fracture-delayed unions or non-unions constitute 10-15% of the approximately 6.5 million bone fractures reported in the US yearly. Osteoarthritis, a degenerative joint disease, affects 27 million Americans, including about 60% of men and 70% of women above 65 years of age. Total joint replacement, while effective as a terminal, irreversible restorative procedure, involves complete removal of the articular joint and associated tissues, and has a finite life expectancy (˜10-15 years) due to failure of the implant-tissue interface. The only biologically based approach available for musculoskeletal repair is the transplantation of cell/tissue grafts, such as tissues of autologous origin. However, this method potentially causes donor site morbidity and is limited by tissue availability. The future of musculoskeletal medicine is thus being developed along two directions:

### Development of Disease Modifying Drugs

The traditional strategy involves a well-defined pathway of laboratory drug development and screening, animal tests, and Phase I, II, and III clinical trials, with final approval of the FDA. In spite of scientific advances in pharmacochemistry, and cellular and genomic biology, current success rates for compounds that reach Phase II and Phase III clinical trials have decreased recently. There are two main reasons for the poor success rates: (1) the lack of reliable toxicity test systems before clinical trials; and (2) the insufficient knowledge of the differences between human and model animals in their responses to drugs. I n particular, species-dependent differences include metabolic pathways, drug sensitivity, tolerance to toxicity, as well as anatomy and physiology. Therefore, human cell-based engineered musculoskeletal tissues (hCEMTs), that mimic native tissue physiology and function, represent a highly promising platform for the assessment of drug toxicities and the prediction of drug efficacy for human application prior to use of the drug in clinical studies. Desirable engineered tissue platforms have some or all of the following features: (1) based on human tissue specific cells; (2) reproducible constructs with custom-designed and biomimetic architecture and infrastructure; (3) three-dimensionality; and (4) production scheme amenable to medium- and high-throughput.

### Engineering of Musculoskeletal Tissues in vitro, ex vivo, or in situ—to Replace or Regenerate, and Restore the Function of Damaged, Injured and Diseased Tissues

This approach involves the combination of biomaterial scaffolds, cells, and signaling from bioactive molecules for new tissue formation. It is desirable to produce hCEMTs to perform these functions. However, currently, no hCEMTs fully replicate or function as native tissues.

Advancements in both of these applications require the in vitro engineering of human tissues. Generally, functional tissue engineering includes cells seeded into three-dimensional (3D) biocompatible scaffolds, with appropriate stimulation by signaling biofactors. As stated above, an ideal hCEMT should be of customizable shapes to accurately mimic native tissue anatomy or to completely fill in the injury defects, as well as possess appropriate internal micro-architecture to facilitate cell/tissue growth. For example, in terms of musculoskeletal tissues, the reported optimal scaffold pore sizes are 100-500 μm for engineered bone, while for engineered cartilage, the optimal pore size is 20-50 μm.

### SUMMARY

Described herein are exemplary methods of fabricating human musculoskeletal tissue. Some methods comprise injecting a liquid material into a musculoskeletal defect site and applying photoillumination to the injected liquid material within the musculoskeletal defect site to cause photocrosslinking of the polymer. The liquid material can comprise a biodegradable and biocompatible polymer, a photo-activated photoinitiator, and human cells capable of producing musculoskeletal tissue. The application of photoillumination causes photocros slinking of the polymer such that the liquid material solidifies into a scaffold having a shape that corresponds to a shape of the musculoskeletal defect site with the human cells encapsulated within the scaffold.

The photoinitiator can be activatable by visible light. For example, applying photoillumination can comprise applying visible light wavelength photoillumination having a wavelength range from about 405 nm to about 490 nm. In some embodiments, the photoinitiator comprises LAP. In some methods, the application of photoillumination causes the liquid material to gelate into an mGL hydrogel in the defect site.

The polymer can comprise natural materials, such as natural gelatin or native collagen, and/or can comprise synthetic polymeric materials. The polymer material can comprise a synthetic, biodegradable material that is not native to humans. The polymer can also comprise or by combined with hyaluronic acid in a combination polymer material. The liquid material can comprise a combination polymer material in addition to the photo-activated photoinitiator and the human cells, wherein the combination polymer material comprises at least one polymer and at least a second material, and wherein the combination polymer material is biodegradable.

In some embodiments, the human cells comprise hBMSCs, hMSCs, or hASCs. In some embodiments, the liquid material comprises a soluble osteoinductive and chondroinductive biofactor, such as BMPs or TGF-βs. In some embodiments, the liquid material comprises a viral vector. In some embodiments, the method further comprises methacrylating the polymer using methacrylic anhydride.

Some methods further comprise dissolving methacrylated gelatin in physiological saline to form a gelatin solution, and then adding LAP into the gelatin solution to form a gelatin/LAP mixture, such that the gelatin/LAP mixture is capable of producing free radicals and photocrosslinking upon visible light exposure. Some such methods further comprise mixing human stem cells into the gelatin/LAP mixture to create an injectable liquid material.

In some methods, the liquid material comprises predifferentiated hMSCs and the method comprises promoting chondral/osseous differentiation of the hMSCs after injection and solidification of the liquid material.

In some methods, the fabricated scaffold has physical properties of stiffness, elasticity, viscoelasticity, hardness, and/or tensile strength that are approximate that of native musculoskeletal tissue at the defect site. The defect site can comprise muscle, bone, and/or cartilage tissue, in any combination, for example.

Also described herein are human cell-based engineered musculoskeletal tissue fabricated using the methods describe herein. In some embodiments, the fabricated musculoskeletal tissue mimics the physiology and function of native musculoskeletal tissue at the musculoskeletal defect site.

Also described herein are systems for fabricating human musculoskeletal tissue in situ. An exemplary system comprises an injection portion and a photoillumination portion. The injection portion is operable to injecting a liquid material directly into a musculoskeletal defect site, with the liquid material comprising a biodegradable and biocompatible polymer, a photo-activated photoinitiator, and human cells capable of producing musculoskeletal tissue. The injection portion can comprise at least a first injector configured to contain the polymer and the photo-activated photoinitator, and at least a second injector configured to contain the human cells, wherein outputs of the first and second injectors merge and join with a needle having an outlet for ejecting and delivering the combined liquid material into the defect site. The photoillumination portion can comprise a light source and a light emitter operable to deliver light to liquid material ejected from the injection portion within the musculoskeletal defect site. The emitted light is sufficient to cause photocrosslinking of the polymer such that the light causes the liquid material to solidify into a scaffold in the defect site having a shape that corresponds to the shape of the musculoskeletal defect site with the human cells encapsulated within the scaffold.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 illustrates a method of projection stereolithography (PSL) fabrication of a computer-aided design (CAD) scaffold encapsulated with lenti bone morphogenetic protein-2 (BMP-2) transduced human-bone-marrow-derived mesenchymal stem cells (hBMSCs).

FIG. 2A is a graph showing medium BMP-2 concentration of cell-laden constructs at culture days 8 and 56.

FIG. 2B includes graphs showing osteocalcin (OCN) and bone sialoprotein II (BSP II) gene expression at culture day 56.

FIG. 2C are ALP stainings of constructs at day 56. Darker/purple staining indicates ALP activity.

FIG. 2D shows osteocalcin IHC at day 56 in vitro culture. Darker/brown staining indicates osteocalcin.

FIG. 3A shows MicroCT images at days 14, 28 after implantation. The circles indicate ectopic bone formation.

FIG. 3B shows macro morphology of constructs in Protein or Gene group at day 56 after implantation.

FIG. 3C illustrates the setting of a three point bending test for testing the physical properties of constructs.

FIG. 3D is a graph showing maximum force recorded during the test shown in FIG. 3C.

FIG. 3E is a graph showing calcium deposition in constructs shown in FIG. 3B at day 56.

FIG. 3F are stainings of the Protein and Gene constructs at day 56.

FIG. 4 is a graph showing medium BMP-2 concentration of cell-laden constructs at culture days 8 and 56. “Protein” represents naïve cells/BMP-2 protein-laden scaffold; “Gene” represents lenti-cytomegalovirus(CMV)-BMP-2/green fluorescent protein (GFP) transduced hBMSC-laden scaffold.

FIG. 5 shows alkaline phosphatase (ALP) staining of constructs at day 56 in vitro culture. Darker/purple staining indicates ALP activity.

FIG. 6 shows osteocalcin IHC at day 56 in vitro culture. Darker/brown staining indicates osteocalcin.

FIG. 7 shows MicroCT images at days 14, 28 and 56 after implantation. Arrows indicate ectopic bone formation.

FIG. 8A shows illustrates a method of visible light (VL)-activated gelation using illumination from a light source emitting light with wavelengths in a range of 430-490 and power/intensity of about 1400 mw/cm².

FIG. 8B are images from a live/dead analysis of hBMSCs encapsulated into methacrylated gelatin (mGL) hydrogel at different depths (⅓ and ⅔ from surface), using ultraviolet (UV)+12959 or VL+lithium phenyl-2,4,6-trimethylbenzoylphosphinate (LAP).

FIG. 9A is a graph showing compressive Young's moduli of mGl hydrogels prepared by different exposure times up to 8 mins.

FIG. 9B is a graph showing collagenase digestion behavior of MGL hydrogels prepared by 2-, 4-, and 8-min light exposure. Wet weight loss of the scaffolds in collagenase solution is shown at different times up to 180 mins.

FIG. 10A is a live/dead assay of hBMSCs cultured in agarose at day 0.

FIG. 10B is a live/dead assay of hBMSCs cultured in MGL hydrogel at day 0.

FIG. 10C is a live/dead assay of hBMSCs cultured in agarose at day 90.

FIG. 10D is a live/dead assay of hBMSCs cultured in MGL hydrogel at day 90.

FIG. 10E is a graph showing MTS assays of mGL and agarose constructs at days 0 and 90.

FIG. 11 includes three graphs showing real-time reverse transcription-polymerase chain reaction analysis of chondrogenic gene expression in hBMSC-laden agarose or mGL hydrogels at day 90 (*p<0.05, **p<0.01). Genes analyzed include Sox 9, collagen type II, and aggrecan. hBMSCs cultured on tissue culture plastic without chondrogenic induction served as control. All values are normalized to control.

FIG. 12A is a graph showing quantitation of glycosaminoglycan (GAG) content deposited on hBMSC-laden constructs after 90 days of chondrogenic culture. Total GAG in a construct. Blank agarose and mGL without cells served as control.

FIG. 12B is a graph showing quantitation of glycosaminoglycan (GAG) content deposited on hBMSC-laden constructs after 90 days of chondrogenic culture. GAG production normalized to DNA content (**p<0.01).

FIGS. 13A-13D are Alcian blue stainings of sGAG in histological section of the hBMSC-laden mGL and agarose constructs after 90 days of chondrogenic culture with Fast Red as nuclear counterstaining.

FIG. 14 is a graph shown mechanical properties of hBMSC-laden mGL and agarose constructs at day 0 and 90 culture in chondrogenic medium (*p<0.05).

FIGS. 15A-15G illustrate a method and apparatus for in vitro assessment of integration of hBMSC-laden mGL and agarose constructs into native cartilage.

FIG. 15A shows a ring shaped cartilage explant.

FIG. 15B shows an inner gap in a cartilage explant filled with hBMSC-laden mGL or agarose.

FIG. 15C shows the composite constructs after being cultured in chondrogenic medium for 6 weeks, ready to be subjected to push-out mechanical testing.

FIGS. 15D-15G illustrate an exemplary push-out test device. The composite constructs are placed in a chamber (4) that holds the cartilage ring in position, allowing push-out access to the inner-implanted component. A motor (1) controls the movement of a plunger (3), and a sensor (2) records the real-time push-out force.

FIG. 15H is a graph showing stress-displacement curves for the agarose/hBMSC and mGL/hBMSC constructs from the push-out test using the device and method of FIGS. 15D-15G.

FIG. 15I is a graph showing failure stress values for different test groups (*p<0.05, **p<0.01).

FIG. 16 is a schematic representation of a multiwell, dual chamber bioreactor system, with a 96 well bioreactor platform shown on the right, and a cross-sectional view of a single bioreactor on the left.

FIG. 17A is a graph showing results of a bioreactor system leak test. Trypsin inhibitor-488 and BSA-555 were simultaneously perfused through the top and bottom space of a bioreactor, respectively, and the percent leakage at different times was estimated based on the bottom-to-top ratio of 488 nm fluorescence readings (Bottom/Top 488) and top-to-bottom 555 nm fluorescence readings (Top/Bottom 555).

FIG. 17B is a graph showing results of a bioreactor system leak test. IL-1β was included in top or bottom stream and perfused for 24 hours. Its concentration in Top or Bottom medium was then measured. There was statistical difference of IL-1β concentration in top and bottom medium. *(p<0.05).

FIG. 18 includes six graphs showing expression of cartilage and bone markers in engineered osteochondral constructs. After 4 weeks of culture in a bioreactor, osteochondral constructs were separated into chondral (Cartilage) and osseous (Bone) components, and each were analyzed for expression of cartilage (Sox9, co12, aggrecan) or bone (RUNX2, osteocalcin, BSPII) markers. Expression levels are normalized to 18S rRNA expression and then to corresponding 2D control expression levels. Expression of cartilage markers was found only in the chondral component, and bone markers in the osseous compartment. *(p<0.05), **(p<0.01).

FIGS. 19A and 19B show histologies of an engineered osteochondral construct. Top: chondral component (CC); bottom: osseous component. FIG. 19A uses Alizarin Red staining and FIG. 19B uses Safranin O/Fast Green staining. Dash lines indicate the border between CC and OC. Bar=100 μm.

FIG. 19C shows an actual macroscopic view of an engineered osteochondral construct. Top: chondral component (CC); bottom: osseous component.

FIG. 20 includes six graphs showing effects of IL-1β treatment on cartilage gene expression in engineered osteochondral microtissue. After treatment of either osseous (Bone) or chondral (Cartilage) component with 10 ng/ml IL-1β for 7 days, cartilage components were analyzed for expression of cartilage markers and MMPs. Untreated constructs were used as controls. Expression levels are normalized to 18S rRNA expression and then to corresponding gene expression under control conditions. *(p<0.05), **(p<0.01).

FIG. 21 includes six graphs showing effects of IL-1β treatment on bone gene expression in the engineered osteochondral microtissue. After treatment of either osseous (Bone) or chondral (Cartilage) component with 10 ng/ml IL-1β for 7 days, bone components were analyzed for expression of bone markers and MMPs. Untreated samples were used as controls. Expression levels are normalized to 18S rRNA expression and then to corresponding gene expression under control conditions.

FIG. 22 includes six graphs showing effects of IL-1β treatment on osseous and chondral MMP secretion in the engineered osteochondral microtissue. After treatment of either osseous (Bone) or chondral (Cartilage) component with 10 ng/ml IL-1β for 1 or 7 days, medium samples collected from the bone or cartilage medium compartment were analyzed by ELISA for the levels of secreted MMP-1, -3, and -13. Values are normalized to those measured under control conditions, which involved untreated osteochondral constructs.

FIGS. 23A-23F illustrate an exemplary method of repairing a cartilage defect using an injectable photocrosslinking gelatin/hyaluronic acid scaffold.

FIG. 23A shows drilling/removal of unwanted material from the defect.

FIG. 23B shows the empty defect after drilling/removal.

FIG. 23C shows the injection of the liquid scaffold containing BMSCs.

FIG. 23D shows in situ curing of the injected material using light.

FIGS. 23E-23F compare results using the illustrated treatment versus results without the treatment.

FIG. 24A shows a defect in a knee joint.

FIG. 24B shows the defect of FIG. 24A after being in situ filled with photocrosslinking gelatin/HA.

FIG. 25 shows testing parameters used in testing the effects of applying various drugs to engineered cartilage tissue (chondrogenesis induced with TGFβ3).

FIGS. 26 & 27 include graphs showing the results of the tests of FIG. 25.

FIG. 28A is a graph showing transduction efficiency at different AAV:cell ratios.

FIG. 28B is a graph showing the MTS assay of constructs with different AAV:cell ratios.

FIG. 28C shows fluorescence microsopy imaging of constructs with different AAV:cell ratios.

FIGS. 29A-29D shows in situ fabrication of gene & cell activated scaffold for the repair of skull bone defect in mice.

FIG. 29A shows preparation of the defect including removal of unwanted material from the defect.

FIG. 29B shows the injection of the liquid scaffold containing polymer, AAV, and cells.

FIG. 29C shows in situ curing of the injected material using light.

FIG. 29D shows the filled and cured defect region.

FIGS. 30A and 30B show micro-CT images of bone defects before and after being treated with different photocrosslinked composite materials.

FIG. 31A shows CAD models of methacrylated poly-D,L-lactic acid/polyethylene glycol/poly-D,L-lactic acid (mPDLLA)-polyethylene glycol (PEG) constructs generated using VL-PSL. (A, B) PDLLA-PEG hydrogels with spherical, cuboidal, and cylindrical architecture.

FIG. 31B shows mPDLLA-PEG constructs based on the CAD models of FIG. 31A.

FIG. 31C shows alphanumeric CAD models of mPDLLA-PEG hydrogels.

FIG. 31D shows mPDLLA-PEG constructs based on the CAD models of FIG. 31C.

FIG. 32 is a graph showing mechanical properties of mPDLLA-PEG/HA scaffolds incubated in HBSS at 37° C. for different times up to 28 days. Difference between every 2 groups is statistically significant.

FIGS. 33A, 33B, 33C, 33E, & 33G show cell viability in VL-PSL generated mPDLLA-PEG scaffolds at 0 days and 28 days after fabrication. Calcein-AM staining (green, live cells).

FIGS. 33D, 33F, and 33H show EthD-1 staining (red, dead cells) in scaffold show the cell viability following fabrication throughout VL-PSL method. Cells were seen to be uniformly distributed at different layers.

FIG. 33I is a graph showing live/dead staining results, cell viability at day 0 and day 28 in control as well as TGF-β3 group.

FIG. 33J is a graph showing MTS assay of constructs at day 0 and day 28 in control as well as TGF-β3 group.

FIGS. 34A-34D are graphs showing real time RT-PCR analysis of gene expression in hASCs at day 28 in 2D tissue culture plate, control group, and TGF-β3 group. All results are normalized to gene expression in 2D culture without chondrogenic induction.

FIG. 35 is a graphs showing hydroxyproline levels in hASC-encapsulated VL-PSL fabricated constructs in the control and TGF-β3 groups at day 0 and day 28. Levels of hydroxyproline in day 0 and day 28 control groups were negligible, and levels in the day 28 TGF-f33 group measured at 21.16±7.15 μg/construct.

FIG. 36A is an immunohistochemical staining for collagen type II in hASC-encapsulated VL-PSL fabricated constructs. Day 0 immediately after fabrication.

FIG. 36B is immunohistochemical staining for collagen type II in hASC-encapsulated VL-PSL fabricated constructs. Day 28 control group.

FIG. 36C is an immunohistochemical staining for collagen type II in hASC-encapsulated VL-PSL fabricated constructs. Day 28 TGF-β3 group. Positive collage type II staining (brown) was only seen in the day 28 TGF-β3 as indicated by arrows.

FIGS. 37A & 37C show glycosaminoglycan (GAG) and proteoglycan content in hASC-encapsulated VL-PSL fabricated constructs visualized by Alcian Blue and Safranin O/Fast Green staining at day 28. Alcian Blue and Safranin O staining, respectively, of control group.

FIGS. 37B & 37D show that negligible amounts of GAG and proteoglycan are detected. Alcian Blue and Safranin O staining, respectively, of TGF-β3 group at day 28, showing strong staining.

FIG. 38 is a graph showing compressive modulus of hASC-encapsulated VL-PSL fabricated constructs measured at culture day 0 and day 28 in the control and TGF-β3 groups.

FIGS. 39A-39D shows Live/Dead staining for four groups utilizing combinations of different polymers after 9 weeks culture in TGF-β3 containing chondrogenic medium: (A) 10% Gelatin, (B) 9.5% Gelatin+0.5% HA, (C)9% Gelatin+1% HA, and (D) 8.5% Gelatin+1.5% HA. In each combination, 20 million/ml hBMSCs were incorporated.

FIGS. 40A-40E are graphs showing expression of chondrogenic genes in real time PCR.

FIG. 40F is a graph showing total GAG assay for the four different polymer combinations.

FIGS. 41A-41D are Safranin O/Fast green stainings for the four different polymer combinations.

FIG. 42 shows a method of medical imaging-guided fabrication of human osteochondral tissues using projection stereolithography for the repair of osteochondral defect.

FIGS. 43A-43D show conversion of an imaging file into 3D models as the template for scaffold printing. FIG. 43A shows micro-computed tomography (micro-CT) imaging of a rabbit condyle. FIG. 43B shows a 3D model being converted from

FIG. 43A. FIG. 43C shows a fabricated solid scaffold. FIG. 43D shows a fabricated porous scaffold.

FIG. 44A shows an exemplary system for delivering polymer and cells materials to a defect site and applying light to the injected material to cure the material in situ.

FIG. 44B shows the distal portion of the system of FIG. 44A, suitable for insertion into a tissue site.

### DETAILED DESCRIPTION

Described herein are methods of fabricating or producing human cell-based engineered musculoskeletal tissues (hCEMTs) using three-dimensional (3D) fabrication technology that involves injectable materials with in situ polymerization/solidification capability and/or solid free-form fabrication. Also described is the usage of hCEMTs for tissue repair and drug testing.

### hCEMTs Produced Using Injectable Materials

Because of the inherent irregular shape of musculoskeletal lesions/defects, it is desired that hCEMTs are capable of filling the defect completely. To be used in minimally invasive procedures without extensive surgical exposure of the diseased tissues and the drainage of body fluid, hCEMTs can be introduced via injection and be retained in situ under aqueous conditions. Using an injection method, the hCEMTs can faithfully reproduce both the biological and physical properties of diseased musculoskeletal tissues.

Among different scaffolds, photocrosslinkable biomaterials are especially attractive because photoactivation is fast and requires relatively mild chemical conditions. However, Irgacure® 2959 (12959), a commonly used photoinitiator to catalyze photocrosslinking, is oxygen sensitive, thus requiring the use of air-tight protective barriers that are incompatible with clinical practice. Without the barriers, the photoillumination may take more than 10 minutes to complete the photocrosslinking progress. Because 12959 activation requires ultraviolet (UV) light, the procedure is also potentially damaging to cellular DNA and compromises cell viability and proliferative ability.

In some methods disclosed herein, a visible light photocrosslinkable hydrogel, comprising biocompatible and/or biodegradable natural or synthetic polymers, such as gelatin, collagen, hyaluronic acid, and/or polyethylene glycol (PEG), is applied to engineer hCEMTs, by utilizing a low toxicity photoinitiator, such as lithium phenyl-2,4,6-trimethylbenzoylphosphinate (LAP), and a visible wavelength photoilluminator.

The term “visible wavelength” means a wavelength between about 390 nm and about 700 nm. Similarly, terms such as “visible light,” “visible wavelength light,” and “visible wavelength photoillumination” mean electromagnetic radiation having a wavelength between about 390 nm and about 700 nm. In some embodiments, a wavelength range of from about 405 nm to about 490 nm can be used, and a corresponding photoinitiator can be used that is activated by light in this wavelength range.

In some methods, the polymers/monomers are first methacrylated using methacrylic anhydride. This modification enables polymers/monomers to crosslink and form a hydrogel when exposed to free radicals. In one example, methacrylated gelatin is dissolved in physiological saline at 10% (w/v), and LAP is then added into the gelatin solution at 0.15% (w/v), allowing production of free radicals upon light exposure. Human cells, such as bone marrow derived mesenchymal stem cells (hBMSCs), are pre-mixed with the methacrylated gelatin/LAP solution, and photoillumination is applied on top of the gelatin/LAP/cell solution. Upon illumination, free radicals from LAP induced by light exposure act to crosslink gelatin into a hydrogel that encapsulates the seeded hBMSCs simultaneously. The hCEMTs produced by this 3D fabrication process can then be used in drug testing, musculoskeletal tissue repair applications, and/or in other applications. hCEMTs produced from such injectable materials have been successfully applied for in vivo repair of osteochondral defects in rabbits and cranial defects in mice, as described in the examples that follow.

### hCEMTs Produced Using Visible Light-Based Projection Stereolithography (VL-PSL)

Scaffolds derived from different materials and with different properties and structures have been fabricated using PSL. Current photoinitiators applied in PSL are not water soluble and must be dissolved in organic solvents. The cytotoxicity of organic solvents thus precludes the use of these photoinitiators in the fabrication of scaffolds from live cell-containing monomer solutions. The requirement of UV activation for the majority of these photoinitiators also carries the risk of generating double-stranded DNA breaks in the encapsulated cells. Other technical hurdles and limitations of current PSL technologies include: (1) lack of temperature control to enhance cell viability; (2) uneven cell distribution and cell clumping within the scaffold, resulting from settling of cells in the static solution during photopolymerization; and (3) presence of organic solvent as carrier for water-insoluble photoinitiators that will compromise cell viability. Therefore, in prior uses of VL-PSL technology, cells are seeded onto the scaffolds after fabrication, rather than incorporated within the scaffold during fabrication. Because the inherent physical barriers imposed by the structure of the scaffold, such cell seeding is inefficient and often incomplete and non-homogeneous. These challenges have greatly limited the application of PSL in live cell-scaffold fabrication.

Thus, in some methods disclosed herein, to produce hCEMTs, novel 3D printing equipment is used with hydrophilic and biocompatible materials to overcome these limitations. 3D printing equipment used in such methods can be include one or more of the following features not present in conventional 3D printing equipment: (1) addition of a temperature controller inside the machine (e.g., inside the building hood) to maintain a desired physiological temperature (e.g., 37° C.) for optimal cell viability; (2) addition of a pump in the machine to circulate and gently agitate the cells/monomer solution to minimize settling of the cells—this feature also allows continuous transition from one biomaterial component to another component, which can be important for multi-component tissue fabrications, such as bone/cartilage and bone/tendon interfaces; (3) placement of the machine in a customized laminar flow hood (e.g., similar to a tissue culture hood) to minimize contamination; (4) use of biomaterials that are photocrosslinkable with visible light illumination, e.g., similar to those used for the injectable materials (see above); and (5) use of a physiological solution, e.g., Hank's Balanced Salt Solution (HBSS), as the solvent carrier for the hydrophilic, photocrosslinkable, and biocompatible monomers.

These technical innovations have thus allowed the successful application of VL-PSL for the production of hCEMTs, suitable as a platform for drug/toxicity testing and as engineered constructs for tissue repair and regeneration. By continuous osteogenic factors stimulation, these VL-PSL hCEMTs have successfully formed matured trabecular bone when implanted into mice.

### Examples of Problems Solved

(1) Tissue constructs fabricated using the 3D photopolymerization technologies described herein are able to fill lesions that have high irregularity and variable depth in anatomy, as often found in clinical musculoskeletal defects, and thus overcome the 3D geometric challenge.

(2) Completely or substantially biodegradable and biocompatible materials may be used, eliminating or minimizing the use of synthetic materials that are not biodegradable, and thus allowing cell remodeling of the extracellular matrix and more efficient and effective integration of the engineered constructs into the surrounding host tissues.

(3) The 3D fabricated hCEMTs described herein may be produced in sufficiently large number and in an identical manner to facilitate medium- to high-throughput in vitro microphysiological platforms (tissue-on-a-chip systems) for drug testing and toxicity screening, thus overcoming the limitations in the development of disease-modifying drugs as described above.

### Exemplary Benefits and Features

(1) Visible light utilized in 3D fabrication eliminates the potential risk of UV light on cells.

(2) The process of hCEMT fabrication is carried out under a biocompatible environment that supports long-term cell survival.

(3) Described methods allow the use of natural polymers, such as gelatin and hyaluronan, to produce hCEMTs in situ at the site of the diseased/damaged tissues for point-of-care musculoskeletal reparative/regenerative therapies.

(4) hCEMTs formed using the described techniques can be made of biocompatible and biodegradable natural extracellular matrix components, such as collagen, gelatin, and hyaluronan, that enhance long-term tissue integration and repair.

(5) hCEMTs may be custom constructed based on computer-assisted design (CAD) reconstruction of clinical images (e.g., from micro-computed tomography, or “micro-CT”) to fit the structural anatomy of the defect sites, which allows better tissue repair and integration.

(6) Large number of identical hCEMTs can be quickly produced.

### Exemplary Applications

The disclosed visible light-based, photocrosslinking fabrication methods can be used to produce cell-encapsulated constructs that develop into functional skeletal tissues, including bone and cartilage, in particular those mimicking the osteochondral junction of the articular joint. As described in the examples that follow, such engineered tissues have been used to examine the effect of bioactive factors that influence skeletal tissue activities, including transforming growth factor (TGF-β1 and -β3), bone morphogenetic protein-2 (BMP-2), interleukin-1β (IL1-β), Indian hedgehog (Ihh), and cyclopamine, with respect to chondrogenesis, hypertrophy, and osteogenesis of human bone marrow derived MSCs (BMSCs). In addition, 3D fabricated hCEMT constructs have been used for the repair of osteochondral defects in rabbits and cranial defects in mice. The results showed that these hCEMTs exhibit effective tissue repair, based on non-invasive medical imaging, and histological and molecular analyses. The hCEMTs produced by means of the 3D fabrication technologies described herein represent a promising product for clinical repair of musculoskeletal defects and for in vitro screening of candidate disease modifying drugs and potential toxicants.

The following enumerated examples provide a more detailed description of some of the many possible applications of the disclosed technologies. Additional information regarding live cell scaffold fabrication techniques, products, and applications can be found in Lin et al., “Application of visible light-based projection stereolithography for live cell-scaffold fabrication with designed architecture,” Biomaterials, 34, 331-339 (2013), which is incorporated by reference herein in its entirety, and in a document by Lin et al. entitled “Single-Step Projection Stereolithographic Fabrication of Gene-Activated Bone Scaffold Encapsulating Human Bone Marrow Stem Cells and Lenti-BMP-2 Viral Vector,” which is appended hereto as part of the disclosure of this application.

### EXAMPLE 1

### PSL Fabrication of Bone Scaffold Encapsulated with Lenti-BMP-2 transduced Human Bone Marrow Stem Cells (hBMSCs)

Large bone defect and nonunion require scaffolds to support local tissue regeneration. To accelerate the process, stem cells and osteogenic factors are often included in the scaffolds. Bone marrow stem cells (BMSCs) represent a promising candidate cell type owing to their osteogenic potential and relative ease of isolation. Visible light-based projection stereolithography (VL-PSL) technology for scaffold fabrication, as described herein, is compatible with concomitant human BMSC (hBMSC) encapsulation and amenable to incorporating medical imaging-derived geometry and internal architecture. Bone morphogenetic protein 2 (BMP-2) is the most commonly used osteoinductive factor, but sustained delivery of bioactive BMP-2 protein is challenging. Ex vivo gene transfer represents an effective method for BMP-2 delivery. hBMSCs, having been previously transduced ex vivo with a lenti-viral vector carrying both BMP-2 and GFP gene expression cassettes (lenti-cytomegalovirus(CMV)-BMP-2/GFP), can be encapsulated into gelatin scaffolds using VL-PSL. The encapsulated vector-transduced hBMSCs can sustainably produce BMP-2 protein to drive spontaneous bone formation by the cell-laden constructs, without BMP-2 addition in vitro or in vivo. Bone formation efficacy in vivo has been assessed using the intramuscular implantation model.

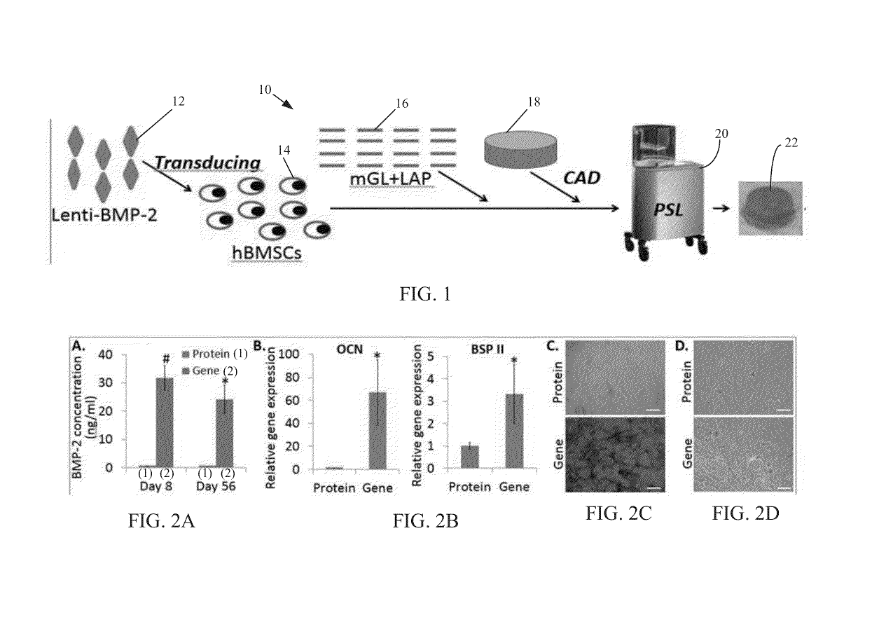

FIG. 1 shows an exemplary PSL fabrication method 10 in which hBMSCs 14 are transduced using Lenti-BMP-2 12 and then encapsulated in a scaffold material 16 and fabricated based on a geometrical CAD model 18 using a PSL apparatus 20 to produce a desirably shaped construct 22 suitable for in vitro use (e.g., BMP-2 ELISA, ALP staining real time PCR, Osteocalcin IHC, etc.) and in vivo use (microCT imaging, H&E staining, 3-point bending test, etc). In an exemplary method, methacrylated gelatin (mGL) was synthesized by reacting gelatin with methacrylic anhydride. hBMSCs can be obtained, for example, from total a joint arthroplasty patient and transduced with lenti-CMV-BMP-2/GFP. Scaffold 16 fabrication using VL-PSL can be carried out using lithium phenyl-2,4,6-trimethyl benzoylphosphinate (LAP) as a photoinitiator. Alkaline phosphatase (ALP) staining (Sigma) and osteocalcin immunohistochemistry (IHC) analysis can be performed.

To test in vivo bone formation, the bioactive scaffolds (2 groups) were individually implanted intramuscularly in both hind limbs in 2 month-old SCID/J mice. Ectopic bone formation was monitored biweekly using micro-computed tomography (micro-CT). Three-point bending tests were performed on the implants to estimate mechanical property of implants.

After VL-PSL encapsulation, in vitro cultured lenti-BMP-2 transduced hBMSCs showed expression and accumulation of BMP-2 protein with high efficiency (FIG. 2A), resulting in increased osteogenic gene expression (FIG. 2B), ALP enhancement (FIG. 2C) and osteocalcin deposition as revealed by immunohistochemistry (IHC) (FIG. 2D). In comparison, naive hBMSCs treated with BMP-2 protein (100 ng/ml) group showed inferior ALP staining, osteocalcin immunostaining, and osteogenesis, likely due to BMP-2 inactivation and dilution after medium change.

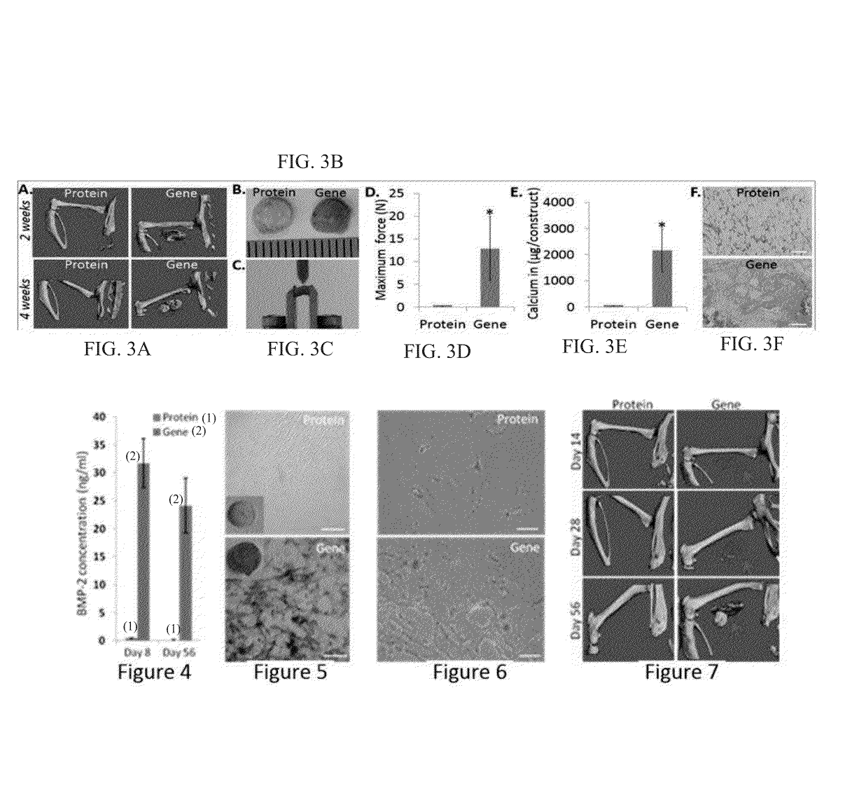

At 14, 28 days post-intramuscular implantation, micro-computed tomography (micro-CT) imaging showed detectable mineralized areas in the lenti-BMP-2 transduced hBMSC constructs (FIG. 3A), suggesting rapid bone formation. Similar areas were not seen in scaffolds in the protein group. After 56 days, constructs in the gene group showed native bone morphology (FIG. 3B). The bone formation was further confirmed by enhanced mechanical properties, such as three-point bending stiffness (FIGS. 3C and 3D) and calcium deposition (FIG. 3E). Hematoxylin-eosin (H&E) staining (FIG. 3F) showed trabecula bone formation in the gene group.

This BMP-2-expressing, hBMSC-encapsulated scaffold fabrication procedure can be applicable for the treatment of bone defects. BMP-2 produced from transduced hBMSCs drove rapid bone formation.

### EXAMPLE 2

### Efficient Bone Formation ex vivo in Constructs of BMP2-Transduced Human MSCs Encapsulated in Gelatin Scaffold Fabricated by Projection Stereolithography

Two current challenges in cell-based bone tissue engineering are: (A) promoting efficient osteogenic differentiation, and (B) fabricating scaffolds that precisely fit the local anatomy and structure. Osteogenesis requires the sustained introduction of osteoinductive biofactors, such as bone morphogenetic protein-2 (BMP-2). However, their short half-lives and rapid clearance in vivo limit their application. Applying viral vectors to deliver BMP-2 has shown therapeutic benefits in healing bone defects. Because of the physical and structural variation in different bones and defects, regenerative repair strategies can include flexible design capabilities to mimic local tissue architecture. Technologies of scaffold fabrication can thus incorporate medical images of the lesion and surrounding tissues as templates. VL-PSL technology for scaffold fabrication is compatible with concomitant human BMSC (hBMSC) encapsulation and is amenable to incorporating medical imaging-derived geometry and internal architecture. This technology may also be used to combine ex vivo gene therapy with biomimetic scaffold fabrication to enhance bone repair. Lenti viral-BMP-2 transduced human bone marrow mesenchymal stem cells (hBMSCs) can be encapsulated within a gelatin-based scaffold using VL-PSL. The ex vivo BMP-2 gene-transduced hBMSCs can be uniformly distributed throughout the scaffolds fabricated with designed architecture, and can sustainably produce BMP-2 protein to drive hBMSC osteogenesis and efficiently form bone upon implantation in vivo without exogenous BMP-2.

In an exemplary method, mGL was synthesized by reacting gelatin with methacrylic anhydride. hBMSCs were obtained from total joint arthroplasty patient and transduced with lenti-CMV-BMP-2/GFP. Scaffold fabrication using VL-PSL was carried out as described herein. ALP staining and osteocalcin immunohistochemistry (IHC) analysis were performed. To test in vivo bone formation, the bioactive scaffolds (2 groups) were individually implanted intramuscularly in both hind limbs in 2 month-old SCID/J mice. Ectopic bone formation was monitored biweekly using micro-CT.

After VL-PSL encapsulation, in vitro cultured lenti-BMP-2 transduced hBMSCs showed expression and accumulation of BMP-2 protein with high efficiency (FIG. 4), resulting in ALP enhancement (FIG. 5) and osteocalcin deposition (FIG. 6). In comparison, naive hBMSCs treated with BMP-2 protein (100 ng/ml) group showed poor ALP staining, osteocalcin immunostaining, and osteogenesis, likely due to BMP-2 inactivation and dilution after medium change. In vivo ectopic bone formation ability was assessed by intramuscular implantation in SCID mice. At 14, 28 & 56 days post-operation, micro-CT imaging (FIG. 7) showed detectable mineralized areas in the lenti-BMP-2 transduced hBMSC constructs, suggesting rapid bone formation. Similar areas were not seen with naïve cells/BMP-2 protein-laden scaffolds.

These VL-PSL fabricated 3D gelatin-based scaffolds, incorporating lenti-viral BMP-2 gene modified hBMSCs, showed efficient BMP-2 production in seeded cells that led to rapid and high-yield osteogenesis in vitro, as well as efficient ectopic bone formation upon intramuscular implantation in SCID mice.

The described BMP-2-expressing, hBMSC-encapsulated PSL scaffold can be applicable for the treatment of large bone defect. The medical imaging-guided fabrication capability of projection stereolithography can further allow such a bioactive scaffold to precisely fit the local anatomy structure without trimming.

### EXAMPLE 3

### Cartilage Tissue Engineering Application of Injectable Gelatin Hydrogel with in situ Visible Light-Activated Gelation Capability in Both Air and Aqueous Solution

Encapsulation of chondroprogenitor cells in a chondrogenically supportive, 3D hydrogel scaffold represents a promising, regenerative approach to articular cartilage repair. In this example, an injectable, biodegradable methacrylated gelatin (mGL) based hydrogel was developed, capable of rapid gelation via visible light activated crosslinking in air or aqueous solution. The mild photocrosslinking conditions permitted the incorporation of cells during the gelation process. Encapsulated hBMSCs showed high, long-term viability (up to 90 days) throughout the scaffold. To assess the applicability of the mGL hydrogel for cartilage tissue engineering, the efficacy of chondrogenesis of the encapsulated hBMSCs was evaluated, using hBMSCs seeded in agarose as control. The ability of hBMSC-laden mGL constructs to integrate with host tissues after implantation was further investigated utilizing an in vitro cartilage repair model. The results showed that the mGL hydrogel, which can be photo-polymerized in air and aqueous solution, supports hBMSC growth and TGF-β3 induced chondrogenesis. Compared to agarose, mGL constructs laden with hBMSCs are mechanically stronger with time, and integrate well with native cartilage tissue upon implantation based on push-out mechanical testing. Visible light photocrosslinked mGL scaffold thus represents a promising scaffold for cell-based repair and resurfacing of articular cartilage defects.

Articular cartilage, found at the surface of the articular joint, has the principal mechanical function of allowing low-friction motion and absorbing and distributing loads. Exposures to forces outside the physiological range, combined with an intrinsic inability of the articular cartilage to manage normal loads, are sufficient to initiate cartilage degeneration and result in osteoarthritis (OA). Due to its avascular nature, cartilage has very limited capacity for self-repair. Currently, there is no effective therapy for OA, with only symptomatic relief until the need for surgical joint replacement. For small cartilage defects, the procedure of microfracture, which involves controlled drilling into the subchondral bone, is commonly employed by surgeons to introduce access to the bone marrow and its constituent cells for cartilage repair. However, the cartilage formed is usually of a fibrocartilaginous nature, and is structurally and mechanically inferior to the native hyaline articular cartilage, thus providing only relatively short-term benefits.

Regenerative medicine approaches that combine cells with scaffolds and signaling factors represent promising, alternative therapies for OA treatment. Chondrocytes and stem cells derived from different tissue sources have been tested as the starting cells population. Bone marrow derived mesenchymal stem cells (BMSCs) are considered a candidate therapeutic cell type for cartilage regeneration because of their self-renewal ability, chondrogenic potential, and other biological characteristics, such as anti-inflammatory activity. Because the articular cartilage is bathed in synovial fluid and is under constant movement, cells for cartilage repair or regeneration need to be delivered to the tissue site encased in a suitable 3D scaffold, such as a hydrogel, in order to limit diffusion of cells as well as to support site-specific proliferation and differentiation. In addition, because of the inherent irregular shape of cartilage lesions, it is desirable that the cell-seeded construct be able to fill the defect completely. To be used in minimally-invasive procedures without drainage of the synovial fluid, it is desirable that the biomaterial be injectable and able to cure under aqueous solution. It is also desirable that the scaffold biomaterial contains cell binding ligands, such as epitopes specific for cell surface integrins, to enhance cell binding and subsequent differentiation and tissue remodeling, as well as integration into the neighboring host tissue. Hydrogel materials can be effective scaffolds because of their high water content, similar to the cartilage extracellular matrix, allowing load transfer from environment to chondrocytes as in native cartilage.

Collagen is a major extracellular matrix component in cartilage, and is thus a suitable biomaterial choice for the fabrication of cartilage tissue engineering scaffolds. However, collagen scaffolds have limitations in terms of concentration (usually <10 mg/ml) as well as stiffness. In addition, hBMSC-seeded pure collagen scaffolds typically contract and lose their original structure, and thus may not be suitable for tissue repair. Gelatin, a denatured form of collagen, is an alternative to native collagen for cartilage repair. Gelatin retains many of the native molecular epitopes for cell adhesion and signal transduction in collagen that are important for the maintenance of the chondrocyte phenotype, and its considerably higher saturation point in aqueous solution (up to 200 mg/ml) serves to increase the structural stability of a gelatin-based scaffold, compared to that formed using collagen.

Different forms of gelatin scaffolds can be used. For example, photocrosslinkable gelatin is a desirable form because photoactivation is fast and requires relatively mild chemical conditions. However, the commonly used photoinitiator (I2959) utilized to initiate photocrosslinking is oxygen sensitive, and thus requires the use of air-tight protective barriers that are incompatible with clinical practice. In addition, I2959 activation requires ultraviolet (UV) light, which is potentially damaging to cellular DNA.

LAP can be used to crosslink polyethylene glycol-diacrylate (PEGDA) for VL-PSL. This crosslinking protocol is capable of rapid generation of cell-laden scaffolds with high cell viability. However, PEGDA scaffolds do not contain cell binding motifs and are not biodegradable, thus limiting cell proliferation and tissue integration. Moreover, an elaborate instrument is required to perform the photocrosslinking. Instead, visible light photocrosslinking can be performed (e.g., with a dental lamp) for the production of a gelatin-based 3D hydrogel scaffold, using mGL/LAP, which is injectable prior to photocrosslinking, biodegradable and biocompatible, exhibits excellent in situ space-filling qualities in air or aqueous solution without the use of protective barriers, and is resistant to swelling and contraction. The applicability of a photocrosslinked gelatin scaffold for cartilage tissue engineering was assessed first by examining the viability, metabolic activity, and chondrogenic activity of hBMSCs encapsulated within the scaffold, as compared to agarose hydrogels. Secondly, the mechanical properties of both cell-seeded mGL and agarose constructs were tested immediately after polymerization/gelation and after 90 days of chondrogenic culture. Finally, the ability of the engineered construct using the mGL scaffold to integrate into host tissue, as a measure of space filling, was investigated employing an in vitro cartilage repair model. The results show that the mGL-based biomaterial scaffold developed in the following study is a promising scaffold for cell-based repair and re-surfacing of acute and/or osteoarthritic articular cartilage defects.

### Exemplary Materials and Methods

### Isolation of hBMSCs

hBMSCs were isolated from human bone marrow, obtained from femoral heads of patients undergoing total hip arthroplasty. Trabecular bone was cored out using curette or rongeur and flushed using 18G hypodermic needles with rinsing medium (α-MEM, 1% antibiotics-antimycotic). The flushed marrow was then passed through 40 μm strainers, and the cells in flow-through were pelleted by centrifugation for 5 min at 300×g. After the supernatant was discarded, cells were re-suspended in hBMSC growth medium (GM, cc-MEM containing 10% fetal bovine serum, 1% antibiotics-antimycotic, and 1.5 ng/ml FGF-2), and then plated into 150 cm² tissue culture flasks. After 4 days of culture, non-adherent cells were washed with phosphate-buffered saline (PBS) and fresh GM was added. Medium was changed every 3 to 4 days. Once 70 to 80% confluence was reached, cells were detached with 0.25% trypsin containing 1 mM EDTA and passaged. hBMSCs isolated from individual patients were validated as capable of osteogenic, adipogenic and chondrogenic differentiation upon stimulation. All experiments were performed with passage 3 (P3) hBMSCs. hBMSCs from 3 patients (54 years old female, 52 years old female and 57 years old male) were used in this study.

### Visible Light-Activated Photocrosslinking of mGL

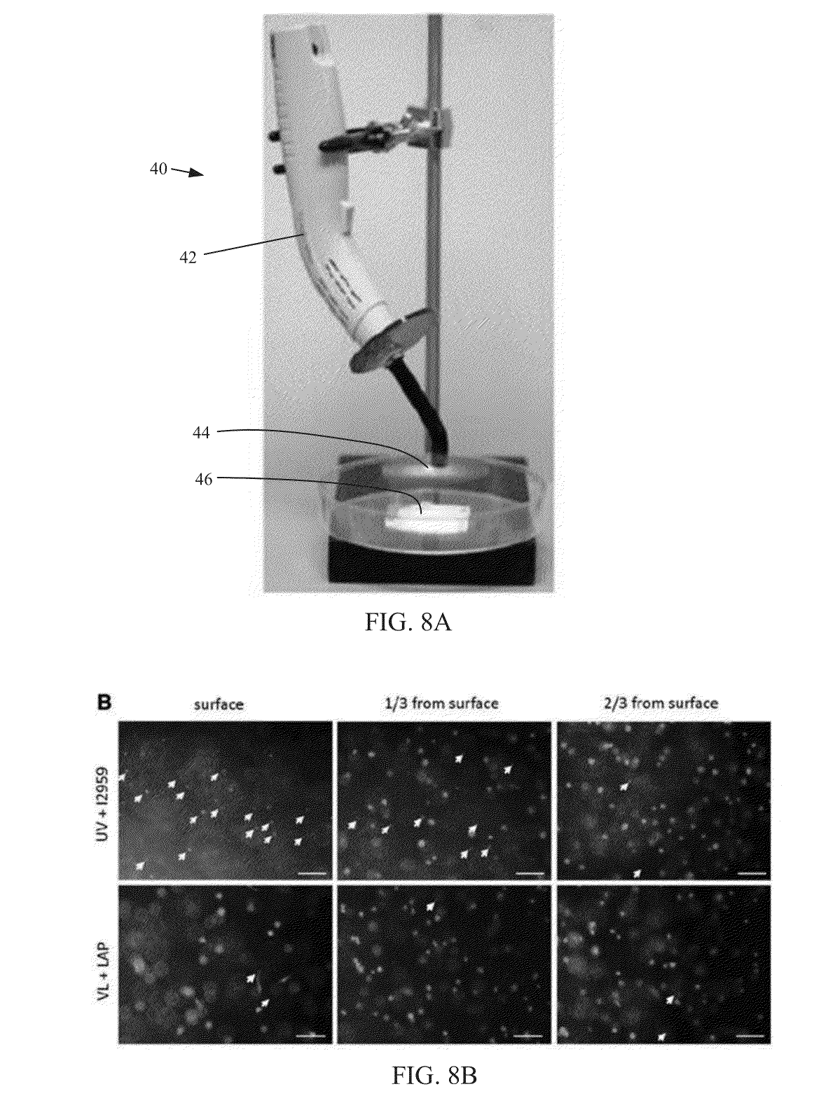

In an exemplary method, mGL was dissolved in Hank's Balanced Salt Solution (HBSS) at 10% (w/v), and after adjustment of pH to 7.4 using 1 N NaOH, the photoinitiator LAP (0.15%, w/v) and 1% antibiotic-antimycotic were added. For scaffold fabrication, the mGL/LAP solution was poured into a 2 mm height silicone mold. As shown in FIG. 8A, the method 40 includes using light source 42 (e.g., a hand-held lamp) to produce a curing light 44 (e.g., visible light at 430-490 nm wavelength and 1,400 mw/cm² power) that is applied directly over the mGL/LAP solution 46 without any covering on top of the solution. The total exposure time was from 1.5 to 8 min. After exposure, the photopolymerized mGL hydrogel was removed from the mold and placed in HBSS for mechanical and degradation testing.

### Analysis of Cell Viability in Different Hydrogels

hBMSCs were suspended in the 10% mGL/0.15% LAP HBSS solution at a final density 4×10⁶/ml and the suspension were poured into a mold with size 20 mm×20 mm×2 mm (L×W×H). After photocuring (4 min visible light exposure as described before), the cell-laden mGL hydrogels were extracted and punched into 5 mm diameter by 2 mm height cylinders. This group was designated as “visible light (VL)/LAP”.

For comparison, another type of hydrogel was produced using the photoinitiator Irgacure 2959 (12959, 0.15% (w/v) final concentration) instead of LAP. mGL solution concentration and cell density were identical to those used in the VL/LAP group. Without any covering to block air, mGL/I2959/hBMSCs solution was subjected to 12 min exposure to long wave length UV light (5000 μW/cm² UV-A). The cell-laden mGL hydrogels were extracted and punched into 5 mm diameter by 2 mm height cylinders. This group was designated UV/I2959.

Cell viability was assessed after 1, 3 and 7 days of culture with the Live/Dead Viability/Cytotoxicity kit and observed by means of epifluorescence microscopy. Four microscopic fields (720 μm×533 μm) per sample were analyzed. Percentage of live cells was calculated based on the number of green stained cells divided by the total number of cells (green and red stained cells, dual stained counted once as dead).

### Mechanical Testing of mGL Scaffolds with Variable Photocrosslinking

Acellular mGL scaffolds fabricated as described above with different exposure times were tested under 10% uniaxial, unconfined compression in an electromechanical tester with a 250 μm load cell. The exact dimensions of cylinders were measured with a caliper, and the cylinders were then placed between stainless steel discs and preloaded to 0.06 μm. The samples were subjected to 10% strain applied at room temperature using a constant rate (0.05%/s). Compressive moduli of mGL scaffolds were determined from the slope of force versus displacement plots.

### In vitro Degradation of Scaffolds

The biodegradability of the mGL scaffolds with different exposure times (2, 4 and 8 minutes) was determined by incubating them with collagenase (1 scaffold in 4 ml 0.05% (w/v) collagenase in PBS (pH 7.4)). After determining the original wet weight (W1), the 8 mm diameter/2 mm height scaffold was completely immersed in the collagenase solution and incubated at 37° C. with shaking at 50 rpm/min, and the wet weight (after blotting) at different time points (W2) was determined. The process was continued until all samples appeared to be completely degraded. The degradation percentage (Q) was calculated as follows: Q=(1-W₂/W₁)×100%.

### hBMSC Encapsulation into mGL Scaffolds for Chondrogenesis

hBMSCs pellets were dissociated and the single cells were suspended within a 10% mGL solution (containing 0.15% LAP) to a final density of 4×10⁶ cells/ml. After curing by 4 min visible light exposure using dental curing lamp, the cell-laden mGL hydrogels were extracted and punched into 5 mm diameter by 2 mm height cylinders. Agarose (2%, w/v) scaffolds seeded with 4×10⁶ cells/ml hBMSCs at identical densities and with identical dimensions served as controls.

The constructs were cultured in chondrogenesis-inducing medium (DMEM with high glucose, 1% penicillin-streptomycin, 0.1 μM dexamethasone, 50 μg/mL ascorbate 2-phosphate, 40 μg/mL L-proline; 1×Insulin-Transferrin-Selenium, 10 ng/ml TGF-β3. Cell viability was assessed after 1, 3, 7 and 90 days of culture with the Live/Dead Viability/Cytotoxicity kit as described before.

### Mechanical Testing of Live Cell-Constructs

After 90 days of culture in chondrogenesis medium, the mechanical property of both mGL and agarose constructs was tested as described above.

### Quantitative Real-Time Reverse Transcription Polymerase Chain Reaction (Real-time RT-PCR)

Total RNA was extracted using TRIZOL-chloroform and purified using the RNeasy Plus mini kit with DNase I treatment. SuperScript III kit was utilized to complete the reverse transcription. Real-time RT-PCR was performed using the StepOnePlus thermocycler and SYBR Green Reaction Mix. Sox 9, Aggrecan and collagen type II (COL2A1) primer sequences are: forward, 5′-AGCCTGCGCTCCAATGACT-3′ & reverse, 5′-TAATGGAACACGATGCCTTTCA-3′; forward, 5′-GGCAATAGCAGGTTCACGTACA-3′ & reverse, 5′-CGATAACAGTCTTGCCCCACTT-3′; and forward, 5′-TTCCGCGACGTGGACAT-3′ & reverse, 5′-TCAAACTCGTTGACATCGAAGGT-3′, respectively. Transcript level of ribosomal protein L13a (RPL13A) was used as endogenous control: forward, 5′-GACACAGGACCACTCATGAAGT-3′ & reverse, 5′-GTGCGGCTGCTTCCATAAG-3′. Gene expression folder changes were calculated using the Comparative CT (AACT) method.

### Glycosaminoglycan (GAG) Quantitation in Constructs

Samples were papain-digested and analyzed for GAG content using the dimethylmethylene blue dye binding assay, with a chondroitin sulfate standard. Digested samples were also evaluated for total DNA content using the Picogreen assay.

### Histological Analysis

Constructs were fixed in 4% paraformaldehyde, paraffin embedded, sectioned at 8 μm thickness, and stained with Alcian Blue/Fast Red using standard protocols for sulfated proteoglycans and cell distribution analysis. Color images were captured using a microscope equipped with digital camera.

### Integration Testing of Cell-Laden Constructs into Cartilage

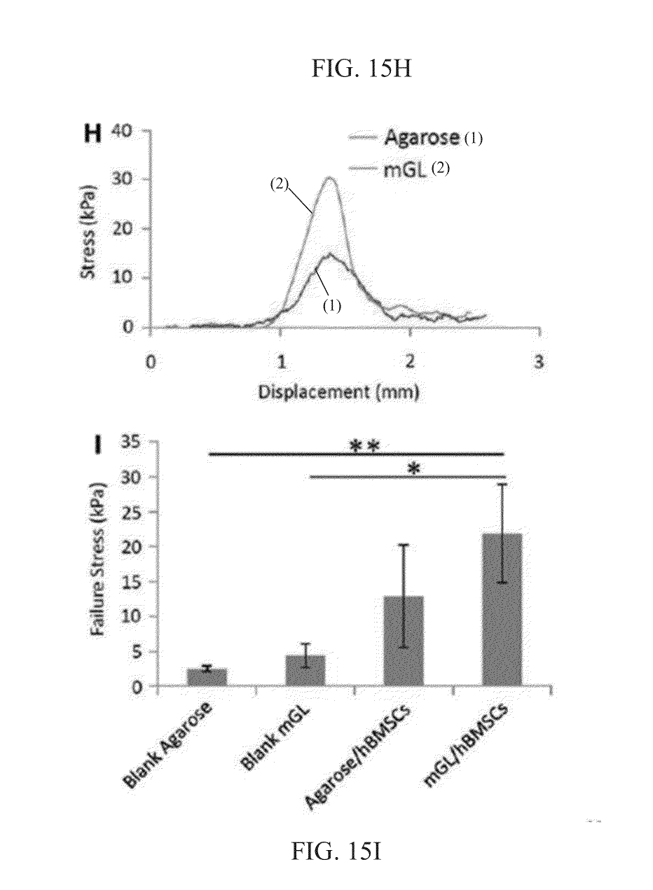

To test the ability of constructs to integrate into the surrounding host tissues, adhesive strength testing of the interface between constructs and native cartilage was employed as shown in FIGS. 15A-15G. Cartilage tissues (about 2 mm thickness) were harvested from the femoral condyles of adult calves and cultured in the chondrogenesis medium for one day. These tissues were then punched into 8 mm diameter cylinders using biopsy punches, and a cartilage ring 30 with a central defect was further created by coring out the center using a 4 mm biopsy punch (FIG. 15A). The cartilage rings 30 were then blot-dried, and hBMSC-laden agarose or mGL solutions (4×10⁶/ml), prepared as described above, were added slowly into the defects until completely filled, followed by gelation by cooling or photocrosslinking, respectively, to form scaffold plugs 32 inside the rings 30 (FIG. 15B), and excess material outside the defect sites removed carefully with a scalpel. The tissue composites 34 (FIG. 15C) were then cultured under chondrogenic conditions, with medium change twice weekly. After 6 weeks, mechanical push-out testing (FIG. 15D) was employed to estimate the interface strength between scaffolds 32 and native cartilage 30. Cartilage samples containing internal constructs were placed on a chamber 4 with a center hole (6 mm diameter, aligned to the defect of cartilage) as shown in FIGS. 15E and 15G. The constructs were pushed out with a hard plastic plunger 3 (FIG. 15F) using the electromechanical tester 1 at a rate of 0.025 mm/sec, and the applied force was recorded using sensor 2 until failure.

### Statistical Analysis

Each study was carried out with three experimental replicates and the results were expressed as the mean ±SD. Significant differences among different groups were determined by two-tailed Student's t-test for two-group comparisons or ANOVA followed by post-hoc analysis for multiple group comparisons. Significance was considered at p<0.05(*) and p<0.01(**).

### Results

### Visible Light-Activated mGL Scaffold Fabrication

Prior to polymerization, the freshly-made mGL/LAP solution had a viscous consistency, spontaneously forming a soft gel at 4° C. that quickly melted at 37° C. The soft mGL gel was readily stabilized through covalent and irreversible LAP-mediated photocrosslinking in the presence of visible light. These properties of mGL show that it is well-suited for in situ tissue repair. As proof-of-concept, a test was performed of the visible light-activated gelation capability of the mGL solution in the context of in situ repair of articular cartilage defects in a tissue specimen obtained as surgical waste following total joint arthroplasty. A portable visible-light dental lamp was able to successfully crosslink mGL, with LAP, in both air and aqueous environment in the absence of O₂ barriers. The capability of crosslinking mGL under aqueous conditions indicates its applicability for tissue repair in clinical settings. A further study of mGL for articular cartilage repair was then performed.

Uniform distribution and survival of cells encapsulated within scaffolds can be important to construct performance in tissue regeneration. Comparison of the viability and distribution of hBMSCs within mGL hydrogel scaffold produced using LAP (visible light mediated crosslinking) or the conventional photoinitiator, I2959 (UV light mediated crosslinking) is shown in FIG. 8B. With LAP, a 4 minute visible light exposure produced a construct with a compressive modulus of 19.8 kPa, whereas using I2959, without the use of an O₂ barrier, a 12 minute UV light exposure was required to produce a construct of comparable mechanical property (18.9 kPa). In constructs produced using LAP and visible light mediated photocrosslinking, cell death was uniformly low (<6%) as shown by Live/Dead staining. In contrast, in constructs fabricated using UV crosslinking, cell death approached ˜100% at the surface (on the side of light exposure) and averaged 22% in regions 600-700 μm from the surface (approximately ⅓ the depth of the construct).

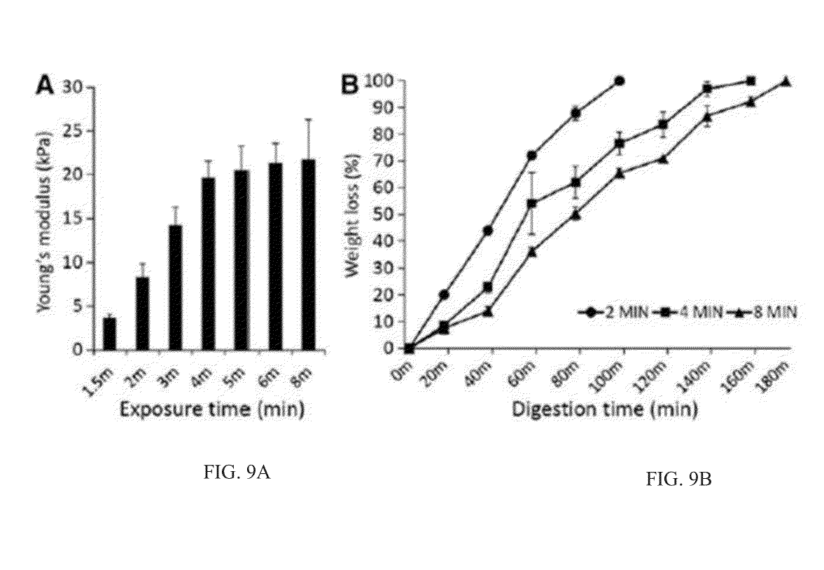

The stiffness of visible light crosslinked mGL scaffolds increased with exposure time, reaching a plateau at 19.8 kPa (compressive modulus) after 4 minutes of visible light exposure. The plateau indicates maximum attainable crosslinking (FIG. 9A). Thus, the scaffold stiffness can be modulated by adjusting the time of visible light exposure. Correspondingly, the time required for scaffold digestion with collagenase also increased with increased crosslinking (FIG. 9B). Even at maximal photocrosslinking, the scaffolds were totally biodegraded within three hours.

### Chondrogenic Differentiation of hBMSCs Encapsulated Within mGL Scaffold

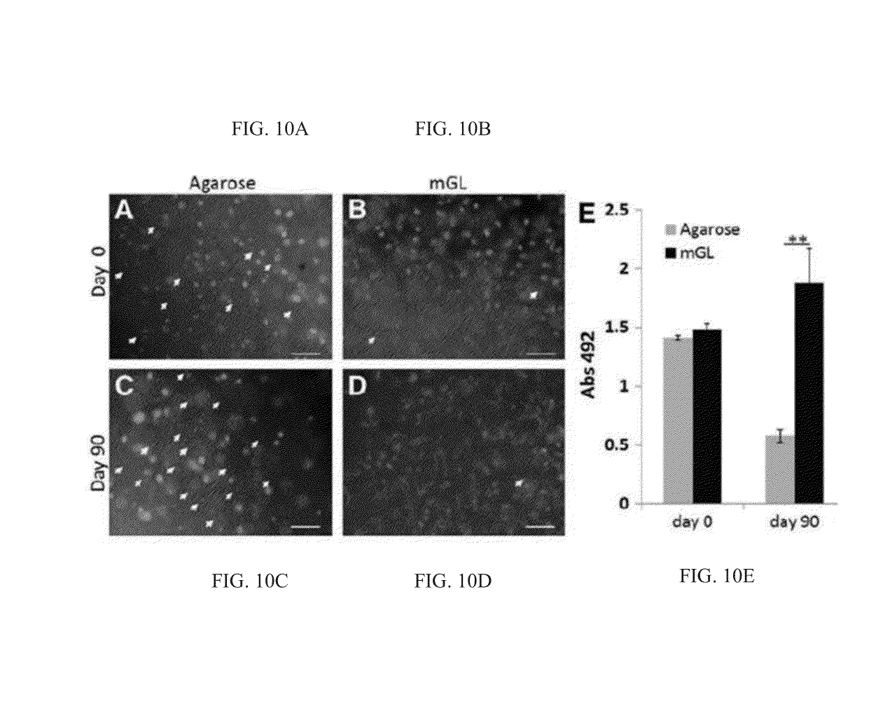

The ability of visible light-crosslinked mGL to support chondrogenic differentiation of encapsulated hBMSCs was examined next. Because the I2959/UV-fabricated mGL scaffold resulted in low cell viability in mGL, agarose was therefore employed as the control as a representative of commonly used biomaterial scaffolds in chondrogenesis studies that also could be delivered via injection for gelation. All constructs (mGL and agarose) were subjected to up to 90 days of culture under chondrogenic stimulation. The high level of viability of the encapsulated cells in photocrosslinked mGL was maintained throughout long-term tissue culture, with viability averaging 92% after 90 days of culture, based on Live (green)/Dead (red) staining (FIG. 10B). FIG. 10D shows the live-dead staining after 90 days. In contrast, cell viability in agarose scaffolds decreased from 75% at day 3 to 40% at day 90 (FIG. 10A). FIG. 10C shows the live/dead staining after 90 days. This observation was supported by metabolic data (MTS assays), which showed that although cell numbers were identical in all constructs at the time of scaffold formation, cell viability was significantly higher in mGL constructs as compared to agarose constructs by day 90 (FIG. 10C). FIG. 10E is a graph showing MTS assays of the mGL and agarose constructs at days 0 and 90.

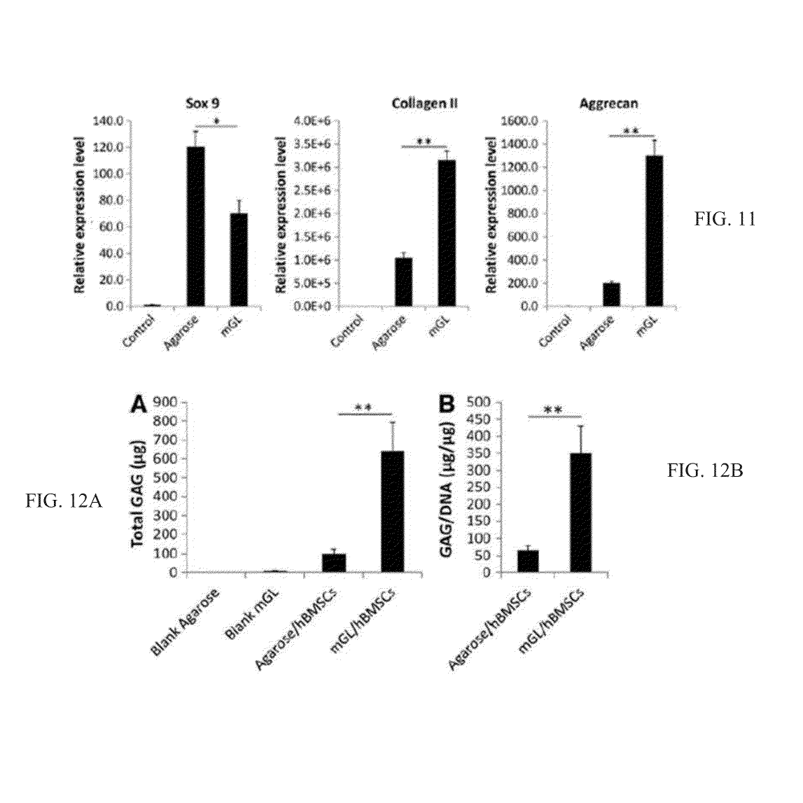

As shown in FIG. 11, after chondrogenic culture for 90 days, substantially higher cartilage matrix gene expression (collagen type II and aggrecan) was seen in mGL constructs versus agarose. This result was supported by biochemical data for GAG production that showed higher GAG levels in mGL scaffolds than agarose scaffolds, calculated on the basis of both per scaffold or per cell after 90 days of chondrogenic culture (FIGS. 12A and 12B). Sox 9 expression was lower in VL/LAP mGL constructs.

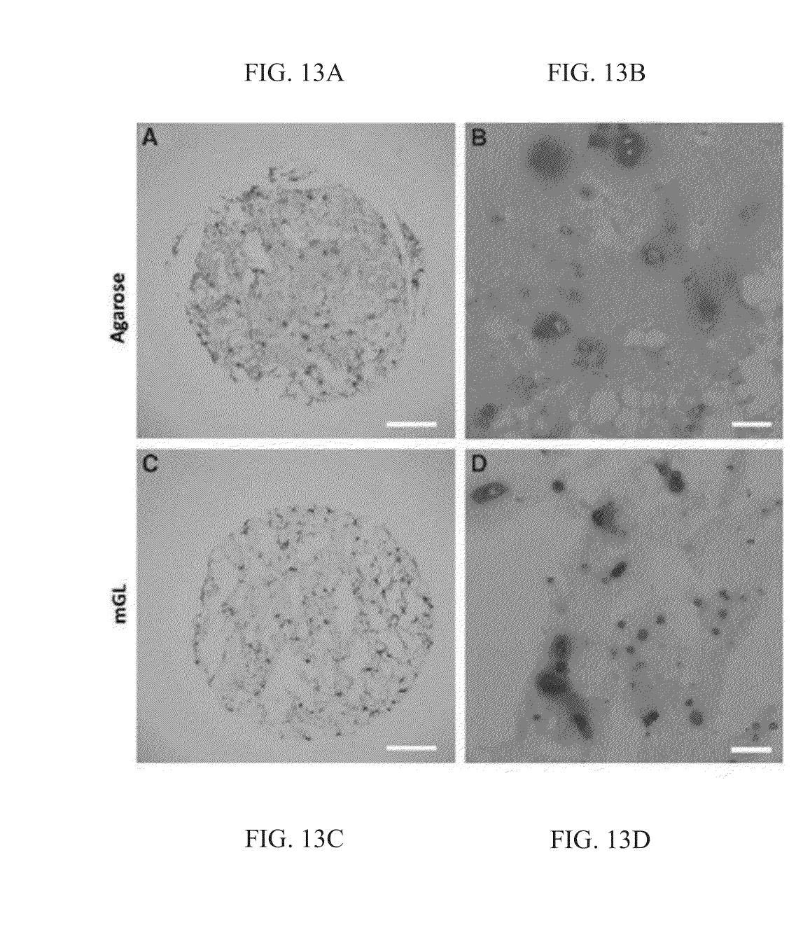

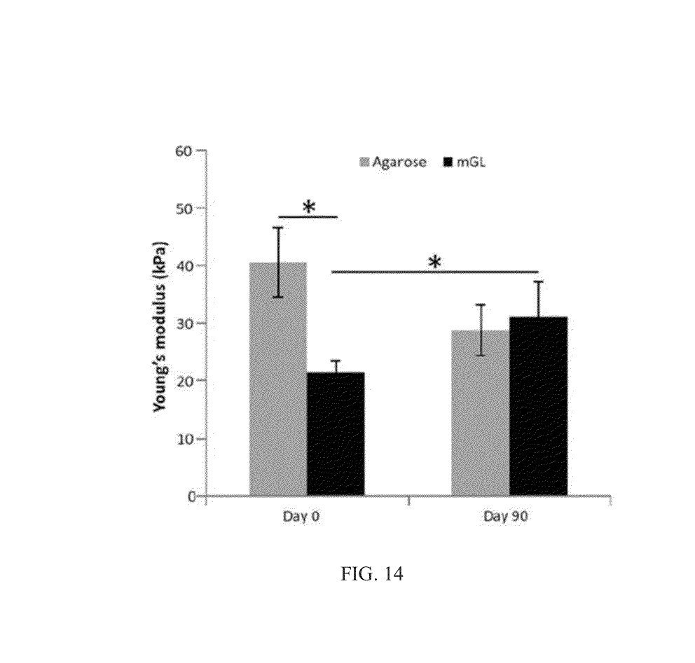

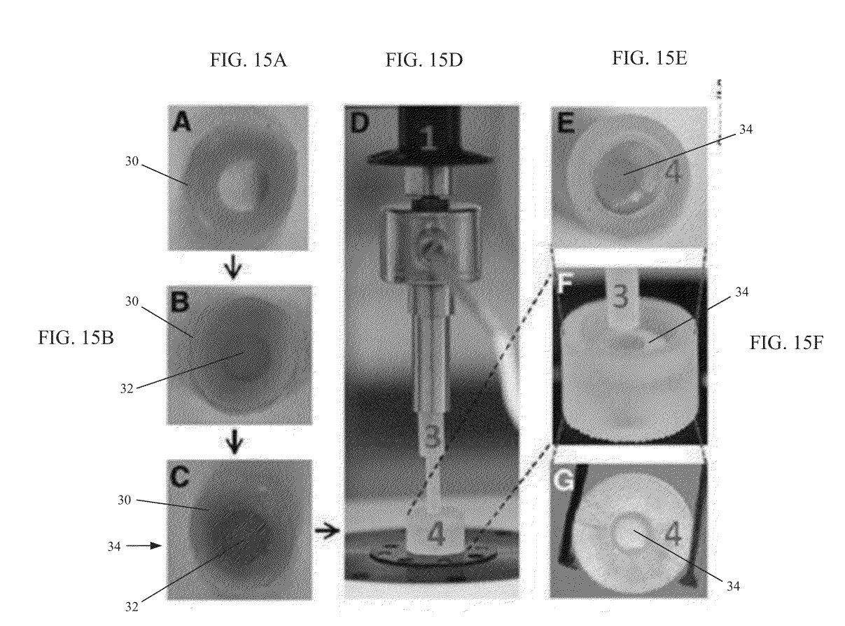

Histological examination revealed increased Alcian Blue stained GAG-rich matrix in the pericellular regions of mGL constructs as compared to agarose constructs (FIGS. 13A-13D), although the overall staining intensity was rather low compared to that typically seen in hyaline cartilage tissue (not shown). Both types of constructs maintained their shape and dimension over the 90 day period. Finally, mechanical testing revealed that immediately after curing, mGL scaffolds had a lower compressive modulus than agarose constructs (FIG. 14). However after 90 days of chondrogenic stimulation, the mGL scaffolds became stronger, attaining a compressive modulus of 31.1 kPa as compared to 21.5kPa at the beginning of culture. In contrast, the stiffness of agarose constructs decreased to 28.8 kPa.

### Integration of mGL Cartilage Construct in an in vitro Cartilage Repair Model

After 90 days of culture, hBMSCs-laden mGL constructs were often bound to each other via neo-matrix. This phenomenon was not observed in the agarose constructs, suggesting that mGL could be specifically well-suited for enhanced tissue integration. To test the potential application of mGL and the process of LAP-initiated visible light crosslinking for in situ articular cartilage repair, an in vitro testing of implant integration was performed. In this study, the center 4 mm diameter zone of an 8 mm diameter disc of full thickness osteochondral articular tissue, harvested from a bovine femoral condyle, was cored out and filled with hBMSC-laden constructs fabricated in situ. After culturing the composite in chondrogenic medium for a pre-determined number of days, the extent of graft-host tissue integration was assessed by measuring the push-out force required to displace/remove the neo-tissue.

FIG. 15H shows the typical stress-displacement curves in hBMSCs-laden mGL and agarose constructs. The force increased as the plunger pressed against the constructs. When the interface between the graft and host tissue failed (the peak force at failure), the curve quickly returned to baseline. All data were collected after 6 weeks of culture in chondro-stimulatory conditions. Without cells, blank mGL constructs placed within native osteochondral tissue had a greater peak force at failure than blank agarose (FIG. 15I), likely due to the formation of some crosslinking between the mGL and native matrix during polymerization. With cells encapsulated, both agarose and mGL had significantly increased peak force at failure stress compared to cell-free constructs, possibly reflecting the influence of the neo-matrix produced. However, although hBMSC-laden mGL constructs displayed, on average, higher push-out strength to failure than agarose constructs FIG. 15I), the difference was not statistically significant.

### Discussion

This example describes a visible light-activated photocrosslinking procedure, using the photoinitator LAP and a portable/handheld light source (e.g., 430-490 nm wavelength range), to prepare injectable mGL hydrogel for cartilage repair. Compared to the commonly used I2959-initiated, UV-based method, the described procedure is able to encapsulate hBMSCs into mGL scaffolds in both air and aqueous solution without the use of any oxygen barriers, and maintain substantially higher cell viability. The mechanical stiffness of the scaffolds is proportional to photocuring time until reaching a platform after 4-minute light exposure. Full biodegradability of the mGL scaffolds is indicated by sensitivity to collagenase digestion. hBMSCs encapsulated in the mGL scaffolds show high viability and chondrogenesis upon stimulation. Compared to agarose, mGL promotes higher chondrogenic ECM gene expression in hBMSCs, as well as GAG deposition and mechanical property enhancement. Because these properties permit direct injection to deliver scaffolds containing cells and/or biofactors into tissue sites in situ using minimally invasive procedures, the mGL scaffold described here is a clinically promising scaffold for articular cartilage repair.

A wide variety of materials and composites have been studied for the production of bioscaffolds for the repair of articular cartilage lesions. Hydrogels are good candidates for cartilage because the microenvironment they provide mimics some properties of native cartilage, including a highly hydrated structure that can transmit biological and mechanical cues, favorable capacity for uniform cell loading, and ready conformation to the recipient tissue site geometry. In addition, hydrogels composed of natural materials can also provide molecular signaling cues that may promote enhanced chondrogenic responses of the encapsulated cells. Most hydrogels, however, lack the required mechanical properties and geometric persistency to repair large articular defects. One solution to this problem is to increase covalent interactions between the hydrogel components through photocrosslinking. Exemplary methods for visible-light based photocrosslinking permit live-cell encapsulation with PEG hydrogels in the process of PSL. The resulting cell-laden scaffolds can be fabricated with greater than 95% cell viability. While PEG may be biocompatible, it is relatively inert and does not support the induction of chondrogenesis as natural matrix macromolecules. Thus, in this study the utility of the visible light photocrosslinking method was assessed for producing constructs based on gelatin, a more bioactive material, for the purpose of articular cartilage wound repair.

The importance of uniformly encapsulating cells within prospective scaffolds for tissue repair has been demonstrated. Integral to this is the need to limit cytoxicity and cell stress during scaffold fabrication. Cells are easily mixed in hydrogels, but the commonly used means of crosslinking using UV light and electron donors such as VA-086, I2959, Darocur TPO, and Eosin Y/TEA/VPL, are all cytotoxic to varying extents, and the stress experienced by the surviving cells may delay, alter or inhibit resident cell differentiation. Moreover, these methods are very oxygen sensitive, and thus require a barrier, such as glass coverslip, to block exposure to air during the gelation process, thus presenting technical complications to in situ gelate and are incompatible with micro-injection to deliver scaffolds with cells or bioactive factors to tissue sites. When the protective barrier is not employed, the curing time of these methods greatly increase, e.g., up to 12 minutes as shown here, greatly impairing cell viability due to long UV exposure (FIG. 8B). In contrast, the visible light crosslinked mGL tested here quickly gelates without the use of a cover, maintains a high percentage of viable cells, and enhances hBMSC chondrogenesis compared to agarose encapsulation, a commonly employed hydrogel for the study of chondrogenesis.

The importance of space filling and geometric persistence is vital to implant success. For example, collagen gels loaded with MSCs have been shown to contract dramatically during differentiation. Gelatin retains many native collagen epitopes that may support chondrogenesis while forming a stable gel. mGL cross-linked via LAP and visible light for 4 minutes attains a compressive modulus comparable to that produced by I2959-initiated UV-crosslinking of 10% mGL, indicating that a similar level of crosslinking has been achieved. Mechanically, constructs prepared using 10% mGL have a Young's modulus of 26 kPa, lower than that of 2% agarose constructs prepared in this study (40 kPa). These values are considerably lower than the 1-2 MPa modulus of native cartilage, severely limiting the utility of the scaffolds in their current formulation for application to articular cartilage repair. Whereas the Young's modulus of agarose constructs decreases with time, mGL-based constructs increase their strength and retain their geometry, and upon placement into native osteochondral tissue show enhanced physical and possible biochemical interaction with the host tissue. Maintenance of construct geometry is indirectly tested here using a scaffold integration test employing an in vitro cartilage repair model, in which space filling of defects and subsequent integration of neo-tissue with host tissue can be measured as a function of the force required to push out the implant. In addition, in both in vitro and in situ articular cartilage simulated wound repair model, the same level of crosslinking is achieved when the process of gelation is performed when the process is carried out submerged in aqueous buffer (not shown), a feature that addresses a significant need in developing an arthroscopic application of this technique.

In summary, the visible light-activated hydrogel described in this study has many properties desirable for an injectable scaffold suitable for articular cartilage repair. Such desirable properties can include any combination of the following: (1) uniform cell distribution at the time of gelation/polymerization and, in particular, delivery of sufficient numbers of viable cells undamaged by harsh chemistry or UV light to enable controlled, robust neo-tissue formation; (2) biocompatibility and chondrogenic stimulatory capacity through its matrix epitope availability and/or growth factor delivery; (3) controlled time and degree of gelation to facilitate defect filling in situ; and (4) a stable, persistent construct geometry during long term differentiation. These properties strongly suggest that the scaffold described here is more suitable for in situ cartilage and osteochondral repair than the hydrogels currently in use. The described scaffold may also be applicable for cell-based articular cartilage repair in vivo. Technical optimization and material modifications to the described formulation may enhance the immediate mechanical properties of the scaffold while retaining its chondrogenic and injectable capacities.

### Conclusions

We have developed a visible light based crosslinking procedure for the preparation of mGL hydrogel. This method allows rapid gelation in air or aqueous solution without any protective barrier, and hBMSC encapsulation with high viability. The mGL scaffolds are injectable, biodegradable, and support efficient chondrogenic differentiation of hBMSCs. Compared to agarose, hBMSC-laden mGL constructs promote enhanced integration between grafts and native cartilage tissue. The mGL scaffold produced by means of visible light mediated crosslinking is thus a promising scaffold for clinical repair of cartilage defects.

### EXAMPLE 4

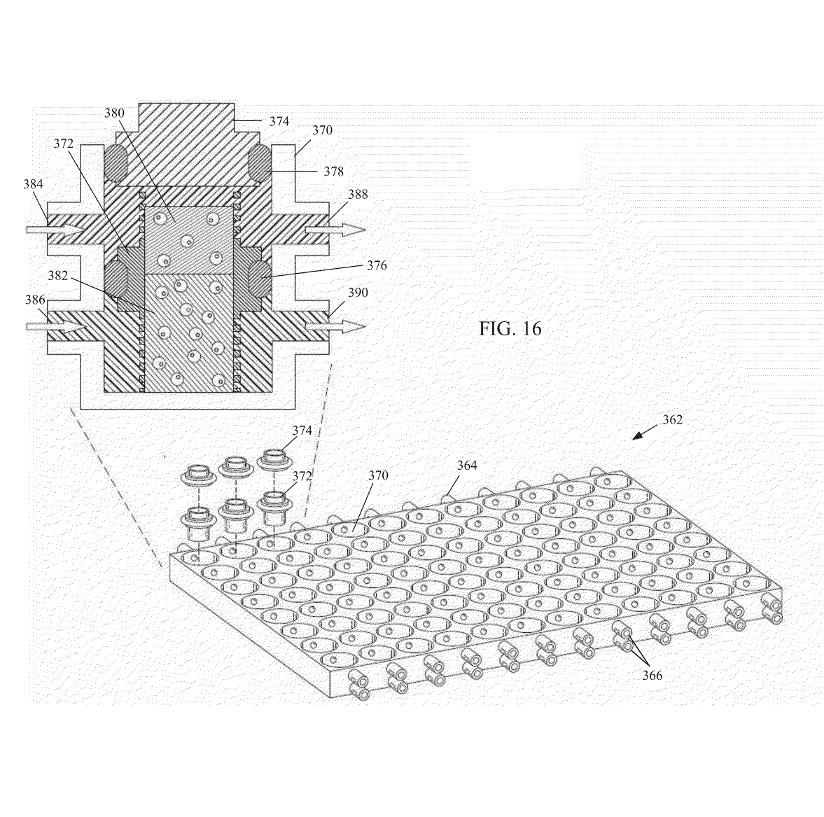

Modular, Microfluidic, Mechanically Active Bioreactor for 3D, Multi-Tissue, Tissue Repair International Patent Application No. PCT/US2014/052348, filed Aug. 22, 2014, which is incorporated by reference herein in its entirety, describes the construction of an in vitro 3D microsystem that models the structure and biology of tissues that are adjacent or contiguous in the body such as the osteochondral complex of the articular joint. Addition information regarding bioreactors and their uses can be found in U.S. Provisional Application No. 62/238,033, filed on Oct. 6, 2015, which is incorporated herein by reference. In certain embodiments, two or more different tissues can be grown adjacent to one another in a bioreactor (e.g., the bioreactor shown in FIG. 16; see detailed discussion in Example 5 below). A bioreactor can be configured with at least two chambers, each independently provided with nutrients and/or fluids, such that different tissues grown in the bioreactor can be fed with different nutrients or fluids. Thus, two or more tissues can be grown adjacent to one another and their interaction(s) can be studied.

In certain embodiments, a bioreactor can include an upper chamber having inlet and outlet ports and a lower chamber having inlet and outlet ports. The inlet ports can be fed by the same or independent sources of biological nutrients, such as liquid cell growth medium, that is perfused through each chamber from the inlet port to the outlet port. A first tissue can be situated in the upper chamber so as to be exposed to the biological nutrients fed through the upper inlet port, and a second tissue can be situated in the lower chamber so as to be exposed to the biological nutrients fed through the lower inlet port. In certain embodiments, one or more additional tissue layers can be situated at an interface that extends partially or completely between the first and second tissues. For example, the additional tissue layer may be a stem cell layer that can differentiate into the first tissue and/or the second layer, and/or that mediates biochemical communication between those layers. In particular examples, the additional layer is a stem cell layer of ectoderm, mesenchyme, or endoderm. In some embodiments, the upper chamber and second chamber can establish substantially separate microenvironments for the first and second tissue by supplying separate media or nutrient flow through the upper and lower inlet ports. Biochemical communication between the separate microenvironments can occur via biochemical signals produced by the additional intermediate layer at the interface instead of via the nutrient media flow.

One exemplary application of the devices, systems and methods described herein is in improved studies of the osteochondral complex and OA. While previous OA studies have focused on the investigation of either the cartilage or the bone component of the articular joint, the osteochondral complex represents a more physiologically relevant target as OA ultimately is a disorder of osteochondral integrity and function. Thus, interactions between both bone and cartilage are central to OA progression, and in studying OA, bone and cartilage are capable of being studied together instead of separately. Thus, the present disclosure describes 3D microtissue constructs including both cartilage and bone, in order to appropriately study the osteochondral environment and OA in vitro.

Different osteogenic and chondrogenic tissue components can be produced using adult human mesenchymal stem cells (hMSCs) derived from bone marrow and adipose seeded within biomaterial scaffolds photostereolithographically fabricated with a well-defined internal architecture. A 3D perfusion-ready container platform, such as a 3D printed platform, can house and maintain an osteochondral microsystem having any combination or all of the following features: (1) an anatomically representative cartilage/bone biphasic structure with a functional interface; (2) all tissue components derived from a single stem cell source, such as an adult mesenchymal stem cell source, to eliminate possible age/tissue type incompatibility; (3) individual compartments to constitute separate microenvironments, for example for the “synovial” and “osseous” components; (4) accessible individual compartments which can be controlled and regulated via the introduction of bioactive agents or candidate effector cells, and tissue/medium sampling and compositional assays; and (5) compatibility with the application of mechanical load or other perturbations, such as chemical, toxicological and other physical perturbations. In certain embodiments, the container platform is dimensioned to fit within the wells of multiwell tissue culture plates, such as 24, 48, or 96 well plates, to perform high-throughput assays. The bioreactor can also have remote imaging capability to allow non-invasive functional monitoring of the bioreactor tissues.

The consequences of external perturbations, such as mechanical injury, exposure to drugs or inflammatory cytokines, and compromised bone quality, on degenerative changes in the cartilage component can be examined in the osteochondral microsystem as a first step towards its eventual application as an improved and high-throughput in vitro model for prediction of efficacy, safety, bioavailability, and toxicology outcomes for candidate disease modifying osteoarthritis drugs (DMOADs). For example, the effects of corticosteroids, anti-inflammatory compounds, or osteoactive agents on the different tissue types, such as bone and cartilage tissue, can be assessed. In addition, drug screening can be performed to identify potential therapeutic agents to treat OA.

In some embodiments, a bioreactor can include a fluidic well plate having dimensions equivalent to those of standard laboratory multi-well plates. The fluidic well plate can have various numbers of wells, such as one well, six wells, twelve wells, twenty-four wells, or ninety-six wells. The wells of the well plates can be arranged in a grid having rows and columns, and a row or a column of wells can be fluidically connected by a first conduit feeding upper portions of each of the wells in the row or column and by a second conduit feeding lower portions of each of the wells in the row or column. Each conduit can begin and terminate at the end of the plate at an inlet or an outlet port.

In some embodiments, a bioreactor can include a fluidic well insert configured to fit tightly within one of the wells of the fluidic well plate and to support biological tissues at an interior of the insert. The insert can include a circumferential flange which seals the insert against the inside surface of one of the wells of the fluidic well plate, thereby separating the respective well into the upper and lower portions fed by the first and second conduits, respectively. The insert can be hollow and thus biological tissues can be housed inside the insert. The circumferential flange can separate an upper portion of the insert from a lower portion of the insert, and each of the upper and lower portions of the insert can include pores through which fluids can flow. The insert can be configured to be situated within a standardized, commercially available well plate.

In some embodiments, a bioreactor can include a lid and an associated support system which is configured to seal the fluidic well plate. The lid can include a micro-mechanical actuator and a force sensor to provide controllable deformation or load to tissue constructs in the well plate. The micromechanical actuator can be associated with and aligned on center with a well of the well plate. The lid can be used with a commercially available well plate with or without an insert situated in a well thereof.

Some embodiments include a modular, microfluidic, multi-tissue, mechano-active 3D bioreactor. A bioreactor can include a microfluidic base, a bioreactor insert, and a mechanoactivating lid assembly. In various embodiments, a base, insert, and lid assembly can be used in various combinations, sub-combinations, or individually. In some embodiments, a base permits direct or indirect interaction of two or more native or engineered tissue types while simultaneously providing separate fluid types to the various tissue types via microfluidic conduits which feed the tissue directly or via biological or physical intermediates within the geometry of standard multi-well plates.

A bioreactor can be amenable and adaptable to common tissue culture practices and devices (e.g., multi-channel pipettes, etc.) and high-throughput formats, depending on the scale of the wells. The insert can divide a single well into upper and lower compartments which do not communicate directly. They may interact indirectly only through the intervening tissue/construct disposed within an inner chamber. Two or more tissues in the inner chamber can interact with each other directly or indirectly while being exposed to two different environments. The dimensions of the inserts can be adapted to fit tissue culture containers of any size and shape. Tissues grown in a bioreactor can be exposed to mechano-activating or other damaging forces. A mechano-activating lid assembly can load and test tissue along a vertical axis while maintaining sterility of the system.

Some embodiments allow growth of an anatomic biphasic structure with a functional interface, and allow growth of each tissue type from a single cell source to eliminate possible age/tissue type incompatibility. Some embodiments include individual compartments to constitute separate microenvironments for the different tissue types, such as for the “synovial” and “osseous” components of a microtissue, each being independently accessible to allow introduction of bioactive agents or candidate effector cells. Some embodiments are compatible with the application of mechanical load and perturbation, as well as with imaging capability to allow for non-invasive functional monitoring.

The described devices, systems, and methods can be used to study bone-cartilage interaction to investigate OA, although their applicability is not so limited. The devices, systems, and methods disclosed can be used to study bone-cartilage interaction to investigate other biological processes or effects, or can be used to study the interaction between other types of tissues.

### EXAMPLE 5

### Stem Cell-based Microphysiological Osteochondral System to Model Tissue Response to Interleukin-1β

Osteoarthritis (OA) is a chronic degenerative disease of the articular joint that involves both bone and cartilage degenerative changes. An engineered osteochondral tissue within physiological conditions will be of significant utility in understanding the pathogenesis of OA and testing the efficacy of potential disease-modifying OA drugs (DMOADs). In this study, a multi-chamber bioreactor was fabricated and fitted into a microfluidic base. When the osteochondral construct is inserted, two chambers are formed on either side of the construct (top, chondral; bottom, osseous) that is supplied by different medium streams. These medium conduits are important to create tissue-specific microenvironments in which chondral and osseous tissues will develop and mature. Human bone marrow stem cell (hBMSCs)-derived constructs were fabricated in situ using the 3D visible light photocrosslinking method described herein and cultured within the bioreactor and induced to undergo spatially defined chondrogenic and osteogenic differentiation for 4 weeks in tissue-specific media. Tissue specific gene expression and matrix production were observed, as well as a basophilic interface, suggesting of the features of a developing tidemark. Introduction of interleukin-1β (IL1-β) to either the chondral or osseous medium stream induced stronger degradative responses locally as well as in the opposing tissue type. For example, IL1-β treatment of the osseous compartment resulted in a strong catabolic response in the chondral layer as indicated by increase matrix metalloproteinase (MMP) expression and activity and tissue-specific gene expression. This induction was greater than seen with IL1-β application to the chondral component directly, indicative of active biochemical communication between the two tissue layers and supporting the osteochondral nature of OA. The microtissue culture system developed here offers novel capabilities for investigating the physiology of osteochondral tissue and pathogenic mechanisms of OA, and serving as a high-throughput platform to test potential DMOADS.

To date there are no proven therapies for the prevention or treatment of OA. Pain relief and visco-supplementation are prescribed to attenuate the symptoms of OA until disease progression significantly impairs joint function and joint replacements are required. The lack of disease modifying OA drugs (DMOADs) may be a function of incongruence between in vitro models of OA and the pathogenesis in vivo and between disease mechanisms in humans and model animals. To overcome these issues, there is increasing momentum to develop human cell-based organotypic models in vitro that functionally represent the osteochondral tissue directly affected by OA.

The development of physiologically relevant models requires an understanding of the tissue architecture, physiology and pathophysiological responses to biochemical (or biophysical) insults. This is especially the case for the osteochondral complex, where the main tissues, cartilage and bone, differ so substantially. Cartilage is comprised of a collagen type II/aggrecan-rich, highly hydrated, viscoelastic, anisotropic matrix that encapsulates chondrocytes within biochemically distinct chondrons. In contrast, bone is comprised of a collagen type I-rich, laminated or woven calcified structure that is much stiffer and encapsulating osteocytes, osteoblasts, and osteoclasts, blood vessels and nerves. The cartilage and bone are intimately connected at the osteochondral junction (OCJ), a highly organized structure that represents a significant challenge to mimic in vitro by tissue engineering. It is comprised of distinct, interacting layers that include (epi-to-diaphyseally) deep zone cartilage, a basophilic tidemark, calcified cartilage, the cement line, and the subchondral bone plate. Interestingly, there is growing evidence of significant biochemical communication between cartilage and bone across the OCJ. In the pathogenesis of OA, changes in the physical linkage between cartilage and bone at the OCJ are critical components of disease progression. These include remodeling of the tidemark, microcracks and fissures in both tissues, and ingrowth from the underlying bone of blood vessels and nerves, all of which may enhance the cartilage-bone crosstalk allowing a better passage of growth factors, cytokines and signaling molecules. These OCJ changes accelerate cartilage degeneration and are associated with joint pain and disease morbidity, pointing to the need of a better understanding of the complex network of interactions between bone and cartilage in OA.

Cartilage and bone exist not only in a different matrix but also in very different biophysical environment. In vivo, there is a steep oxygen gradient from bone (essentially normoxic) to cartilage (extremely hypoxic). These differences are reflected in the in vitro culture systems often used to maintain chondrocytes and osteoblasts. Chondrocytes are best maintained in a “starved” environment: low glucose, serum-free medium supplemented with pyruvate and abundant matrix precursors or -enhancing molecules (proline and ascorbate) in hypoxic conditions. On the other hand, osteoblasts are maintained in high glucose, serum containing medium supplemented with β-glycerol phosphate and vitamin D3 in normoxic conditions.

With these fundamental environmental differences between chondrocytes and osteoblasts, it is not surprising that OA elicits specific responses from each tissue. OA disease progression is most frequently characterized by a net loss of cartilage matrix that results from an imbalance between cartilage matrix degradation and synthesis by chondrocytes in the cartilage. Progressive chronic destruction of articular cartilage is the most obvious characteristic of OA, and the etiology of the disease is believed to be at the intersection of genetics and abnormal mechanical forces. Therefore, the primary locus of the disease is traditionally presumed to be the cartilage, and as a result, most in vitro OA models focus exclusively on cartilage to study OA disease mechanisms and therapeutic intervention. However, there is increasing evidence from in vivo and clinical studies that subchondral bone lesions may precede cartilage degeneration, implying that OA is an osteochondral disease and possibly bone dependent. In addition, it has been often reported that the health of mature articular cartilage in vitro is positively impacted by the presence of subchondral bone.

Despite these observations, most in vitro OA research has not taken into account the effects of bone-cartilage interactions, focusing primarily on cartilage alone. This may account for the dearth of new therapeutics for the prevention and treatment of OA. The development of a model system of osteochondral tissue using human cells in a physiologically relevant environment that can accurately replicate in vivo osteochondral tissue homeostasis and pathophysiology may lead to greater predictive power in the development of DMOADs. Exemplary challenges in developing such a system include: (1) mimicking or inducing production of appropriate extracellular matrix so critical to the function of cartilage and bone, (2) replicating the tissue architecture, (3) reconciling the different growth and maintenance conditions of bone and cartilage while promoting their interaction with each other, and (4) replicating the biomechanical environment known to be essential to cartilage and bone health.

Current in vitro models to investigate bone-cartilage interactions are mostly limited to cell co-culture systems in which bone and cartilage cells are both exposed to the same medium, arguably a very distant condition from the in vivo environment. Herein is described the development of an human MSC-based osteochondral tissue engineered using the photocrosslinking method described herein, and contained within a bioreactor designed to accommodate the biphasic nature of the osteochondral tissue by creating two separate compartments for the “chondral” and “osseous” microenvironments. These are separated only by the tissue itself and are supplied by a microfluidic system. The two microenvironments can be independently controlled and regulated via introductions of bioactive agents or candidate effecter cells, and the medium can be individually sampled for compositional assays. A gradient of tissue specific nutrients and conditions may be required for the formation and maintenance of the osteochondral tissue. Furthermore, induction of an OA-like condition in the engineered osseous or the engineered chondral component alone may induce a corresponding OA-like response in the other component. In this study, distinct chondral and osseous zones are generated within the same construct by controlling the different media exposures within the bioreactor. Then, an OA-like response is induced by exposing the osseous or chondral compartments to the pro-inflammatory cytokine (IL-1β) and the intervening changes in expression and secretion from both the engineered chondral and osseous components are assessed.

### Exemplary Materials and Methods

Bioreactor design and fabrication. FIG. 16 is a schematic representation of a multiwell, dual chamber bioreactor system, with a 96 well bioreactor platform 362 shown at the lower right, and a cross-sectional view of a single bioreactor shown at the upper left. The multi-well platform 362 includes a plurality or rows of eight wells 370 that are in fluid communication from one inlet/outlet pair 364 across the row of wells 370 to an opposite inlet/outlet pair 366. Each well 370 is configured to receive a bioreactor insert 372 and a sealing lid 374 (the lid can be replaced with and/or incorporated into a mechanical actuator or piston that applies a mechanical loading pattern downward on the tissue/fluid in the bioreactor). The insert 372 is sealingly engaged with the inner surfaces of the well 370 via an o-ring 376 to form separate upper and lower fluid flow chambers. The lid 374 is also sealingly engaged with the inner surfaces of the well 370 via another o-ring 378 to prevent fluid escaping from the well. The insert 372 can contain at least two layers of biological material, such an upper layer 380 and a lower layer 382 as shown. The upper layer 380 can comprise a chondral construct and/or the lower layer 382 can comprise an osseous construct, for example. One or more additional layers, such as an intermediate layer, can also be included. An intermediate mesenchymal layer can be included, for example. Each well can have two opposing upper inlet/outlets 384 and 388, which allow a first fluid to flow through the upper chamber to interact with the upper layer 380, and two opposing lower inlets/outlets 386, 390, which allow a second fluid to flow through the lower chamber to interact with the lower layer 382. The first fluid can comprise a chondrogenic medium and/or the second fluid can comprise an osteogenic medium, for example.

As illustrated in FIG. 16, the first fluid can enter at 384 and then pass laterally through perforations in the insert 372 to enter the upper layer 380 laterally. The first fluid can then exit the upper layer 380 laterally through the perforations in the insert 372 before exiting the bioreactor at 388. The perforations can extend circumferentially around the insert 372 such that the first fluid can flow around the upper layer and can interact laterally with the upper layer from all lateral sides. Some of the first fluid can also flow over the top of the upper layer and perfuse into and out of the upper layer from its upper surface. Similarly, the second fluid can enter at 386 and then pass laterally through perforations in the lower portion of insert 372 to enter the lower layer 382 laterally. The second fluid can then exit the lower layer 382 laterally through the perforations in the insert 372 before exiting the bioreactor at 390. The perforations can extend circumferentially around the lower portion of the insert 372 such that the second fluid can flow around the lower layer and can interact laterally with the lower layer from all lateral sides. More information regarding the bioreactor shown in FIG. 16 and its possible uses can be found in WO 2015/027186, published on Feb. 26, 2015, which is incorporated by reference herein in its entirety.

Isolation of hBMSCs. hBMSCs were isolated from the femoral heads of patients undergoing total joint arthroplasty, cultured and expanded as previously described (Caterson, 2002; Song, 2004). Briefly, bone marrow was flushed out from the trabecular bone of the femoral neck and head using an 18-gage needle and re-suspend in Dulbecco's Minimal Essential Medium (DMEM). The suspension was filtered through a 40 μm strainer and the flow-through was centrifuged at 300 g for 5 minutes. After the supernatant was discarded, the pellets were suspended using growth medium (GM, α-MEM containing 10% fetal bovine serum (FBS, Invitrogen), 1% antibiotics-antimycotic, and 1.5 ng/ml FGF-2 (RayBiotech, Norcross, Ga.)), and then plated into 150 cm² tissue culture flasks at a density of 20-40,000 nucleated cells/cm³, medium was changed every 3 to 4 days. Once 70% to 80% confluence was reached, cells were passaged. The colony formation and tri-lineage mesenchymal differentiation capacity of hBMSCs was validated before use (data not shown). All experiments were performed with passage 3 (P3) hBMSCs, from 3 patients (3 female patients 44, 52 and 72 years old) which were pooled for use in this study.

Preparation of photoinitiator LAP. The photo-initiator lithium phenyl-2,4,6-trimethylbenzoylphosphinate (LAP) was synthesized as described by Fairbanks et al.

Preparation of methacrylated gelatin (mGL) and hyaluronic acid (mHA). mGL was synthesized by reacting gelatin with methacrylic anhydride (MA) in water. mHA was prepared using sodium hyaluronate powder (research grade, MW ˜66 kDa, Lifecore). Both mGL and mHA are lyophilized and stored in a desiccator for future use.

Bioreactor system leak test. To test medium leakage between (1) the chamber wall and the insert, and (2) the insert and scaffold material, the insert was filled with 10% mGL/0.15% LAP in HBSS and cured by photocrosslinking. Alexa Fluor® 488-conjugated soybean trypsin inhibitor, (TI488, 21kd, Molecular Probes, CA) and Alexa Fluor® 555-conjugated albumin from bovine serum (BSA), (BSA555, 65kd, Molecular

Probes) were diluted in HBSS individually at 10 μg/ml and then perfused through the top and bottom of bioreactor, respectively, at 0.001 ml/minute. At different time points, effluent from the upper and lower medium conduits was collected and the fluorescence intensity at both wavelength measured using a microplate reader (Synergy HT, BioTek, Winooski, Vt.). Leaking between top and bottom conduits was estimated by the ratio of TI488 (Bottom)/TI488 (Top), and BSA555 (Top)/BSA555 (Bottom). Due to the permeable nature of gelatin scaffold used as the scaffold model, leaking was assayed for 24 hours only.

Fabrication of naïve osteochondral constructs in vitro. P3 hBMSCs were pelleted and drained completely in order to prevent the unwanted dilution of polymers. Chondrogenic cell suspension: hBMSCs were re-suspended in 10% mGL/1% mHA/0.15% LAP (w/v) HBSS solution (pH adjusted to 7.4) at a final density of 20×10⁶/ml (Chondrogenic suspension). Osteogenic cell suspension: hBMSCs were re-suspended in the 10% mGL/1% Hydroxyapatite/0.15% LAP (w/v) HBSS solution (pH was adjusted to 7.4) at a final density 20×10⁶/ml. Osteochondral construct preparation: First, the insert was placed within a hollow cylindrical well to prevent suspension leaking from the pores in the insert. Second, 600 of osteogenic suspension was pipette into the insert and cross-linked with photoillumination as described in Example 3. After photocrosslinking, the osseous construct was removed from the chamber. Third, 300 of chondral suspension was added on the top of osseous construct within the same insert, and cured for another 2 minutes. Studies has shown cell viability within the scaffold >90% after photocrosslinking (data not shown). The second round of crosslinking had the added benefit of bonding the osseous and chondral layers together as well, and the fabrication of the osteochondral construct within the insert created a tight seal.

Culture of osteochondral constructs in vitro. The inserts with naïve osteochondral constructs were placed into the microfluidic plate as shown in FIG. 16. Chondrogenic medium (CM) was supplied through the upper conduit while osteogenic medium (OM) through the bottom conduit at a flow rate of 1 μs. The following formulae were used for the differentiation media: OM (GM supplemented with 10 ng/ml BMP-2 (PeproTech, Rocky Hill, N.J.), 1% L-alanyl-L-glutamine (G1utaMAX), 10 nM dexamethasone (Dex), 0.1 mM L-ascorbic acid 2-phosphate (AsA2-P), and 10 mM beta-glycerophosphate (β-GP); CM (DMEM supplemented with 10 ng/ml TGF-β3 (PeproTech), 1% ITS, 50 μM AsA2-P, 55 μM sodium pyruvate, 23 μM L-proline). The perfusion rate was 0.001 ml/minute and used syringes were replaced with syringes and new medium every 3 days. After 4 weeks of differentiation, engineered osteochondral tissues were collected for validation using real time PCR and histological analysis, or treated with IL1-1β.

Real time PCR. Chondral and osseous constructs were collected separately. To avoid the potential contamination, the bonded margin of each construct was cut away using a razor blade. Total RNA was extracted using Trizol (Invitrogen) following the standard protocol and purified with the RNeasy Plus mini kit (Qiagen, Hilden, Germany). SuperScript III kit (Invitrogen) was utilized with random hexamer primers to complete the reverse transcription. Real-time RT-PCR was performed using the StepOnePlus thermocycler (Applied Biosystems, Foster City, Calif.) and SYBR Green Reaction Mix (Applied Biosystems). Sox 9, Aggrecan (ACN) and collagen type II (COL2A1), RunX2, Osteocalcin (OCN), Bone sialoprotein (BSP II) expression were analyzed. Monolayers of hBMSCs cultured in GM on 2D tissue culture plastic were used as negative controls. Transcript level of 18S rRNA was used as endogenous control: forward, Gene expression folder changes were calculated using the Comparative CT (ΔΔCT) method.

Histological analysis. Intact engineered osteochondral tissues were fixed in buffered 10% neutral buffered formalin (Fisher Scientific, Pittsburg, Pa.) for 7 days, dehydrated, embedded in paraffin with 10-μm sections cut from each sample and stained with Hematoxylin and eosin (H&E) for histological observation. Safranin O/fast green staining was used to detect the GAG deposition.

IL-1β treatment in engineered micro-osteochondral constructs. After 4 weeks of differentiation, engineered the osteochondral constructs were treated with IL-1β 5 ng/ml, R&D) on the chondral or osseous sides only to investigate the cell/neo-tissue response to pro-inflammatory cytokines and possible communication through the osteochondral construct. The media used in this test were CM without TGF-β3 (chondral) and OM without BMP-2 (osseous) both supplemented with 5 ng/ml There were experimental groups: (1) CM/GM, (2) CM+IL1-1β/GM, (3) CM/GM+IL1-1β. The treatment lasted 7 days, with effluent medium collected and frozen at 1, 7 days for ELISA. After 7 days, the osteochondral constructs were bisected into the chondral and osseous halves and processed for gene expression analysis as described before. In addition to tissue specific gene expression, matrix metalloproteinase 1, 3, 13 were also analyzed.

Enzyme-linked immunosorbent assay (ELISA). Media was collected separately from chondral and osseous constructs, cleared of cell debris via centrifugation (1000×g), and analyzed via IL-1β (Abcam, Cambridge, Mass.), MMP-1 (R&D), MMP-3 (Abcam, Cambridge, Mass.), and MMP-13 (Abcam) ELISAs according to the manufacturers' instructions.

Statistical Analysis. Results are expressed as mean±standard deviation (SD). Significant differences were determined with ANOVA followed by a Bonferroni post hoc analysis for multiple group comparisons using SPSS Statistics 21 (IBM, Armonk, N.Y.). Significance was determined at p<0.05(*) and p<0.01(**).

### Results

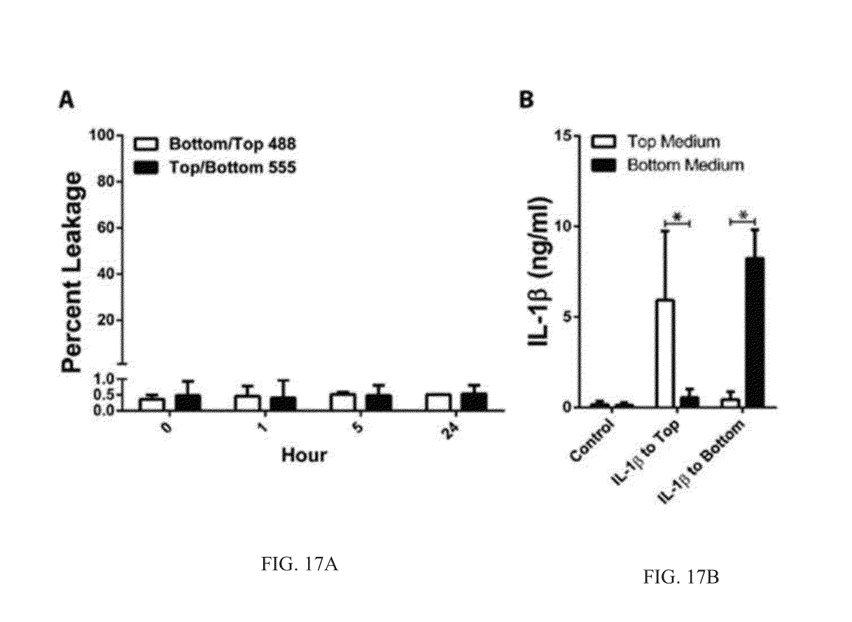

Bioreactor system leak test. The robustness of the microbioreactor was tested by assessing the extent of leakage 2 molecules perfused independently in the upper and lower medium conduits: (1) trypsin inhibitor (21 kD) with a molecular weight similar to the two commonly used osteoinductive (BMP-2, 26 kd) and chondroinductve (TGFβ3, 25 kd) factors, and (2) BSA (65kD), the most abundant protein in serum. As shown in FIG. 17A, after 24 hours of perfusion, the extent of mixing between top and bottom was <1%, indicating there was minimal medium exchange through the interfaces between the chamber wall and the insert, and between the insert and scaffold-only construct. These results were further confirmed by ELISA assay for IL-1β in both medium conduits during the IL-1β test (FIG. 17B).

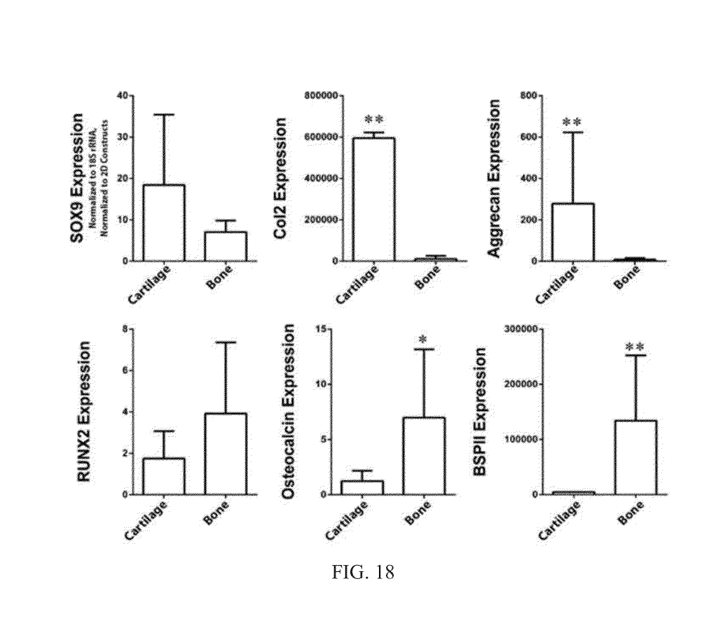

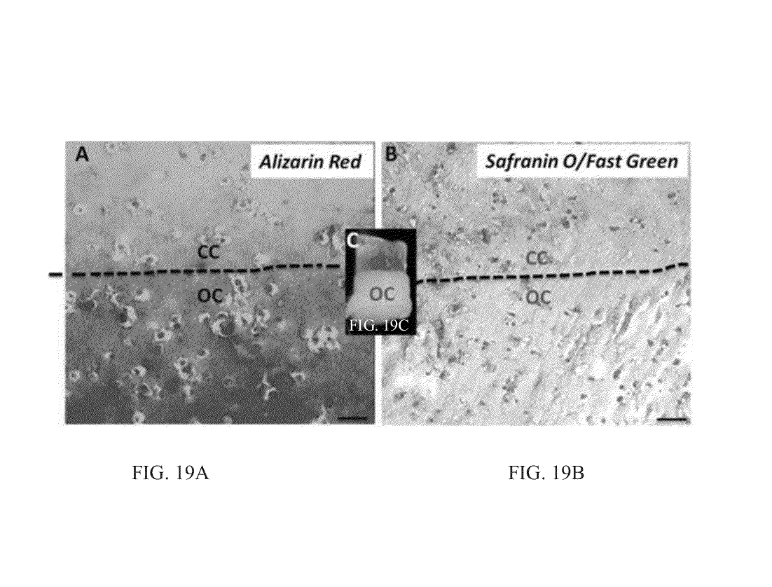

Differentiation of engineered osteochondral construct. The naive hBMSCs seeded within tissue-specific scaffolds in the freshly fabricated osteochondral construct were induced to differentiate using CM in the top stream and OM in the bottom stream. Chondrogenesis was anticipated in the upper, chondral half of the construct and osteogenesis in the bottom, osseous half. After 4 weeks of differentiation, biphasic osteochondral constructs were produced. As shown in FIG. 18, cells in the chondral half showed enhanced expression of chondrogenic genes, including Sox 9, aggrecan and collagen type II as compared to those in the osseous half, while the cells in the osseous half had higher expression of osteogenic genes, including RunX2, osteocalcin and BSP II. Monolayers of hBMSCs cultured in GM on 2D tissue culture plastic were used as negative controls. Histological staining with Alcian Blue/Alizarin Red revealed high matrix GAG content in the upper chondral half than the osseous half (FIG. 19B); although the amount of calcium deposition in the osseous half was not detectable. Taken together, these results strongly indicate a spatially defined, biphasic differentiation of these engineered osteochondral constructs, with the chondral component undergoing more characteristic differentiation. In addition, H&E staining revealed a distinct, <100 μm wide basophilic band in the interface between the chondral and osseous halves, potentially indicative of a developing tidemark (FIG. 19A). FIG. 19C shows a macroscopic view of the engineered osteochondral construct.

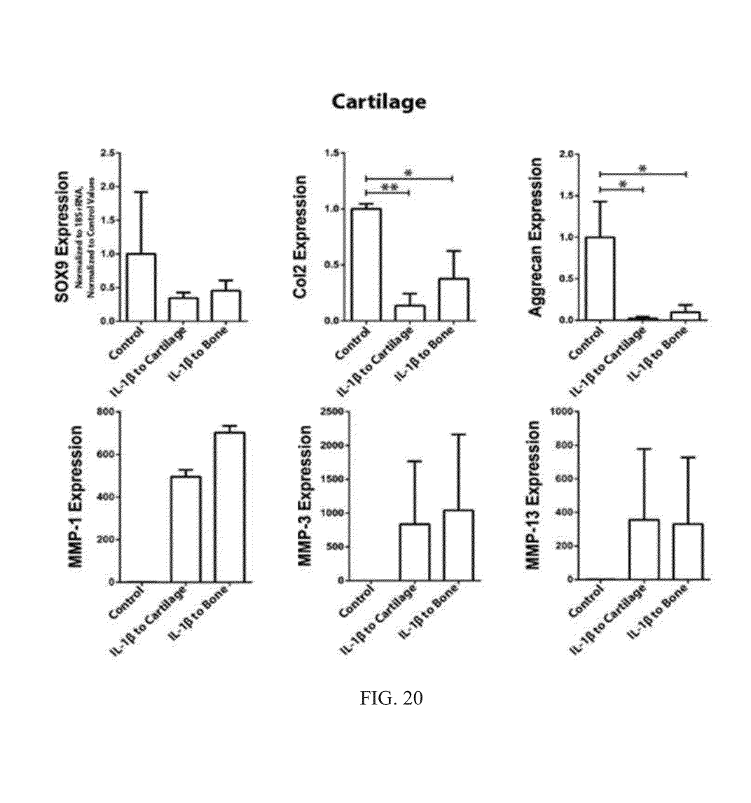

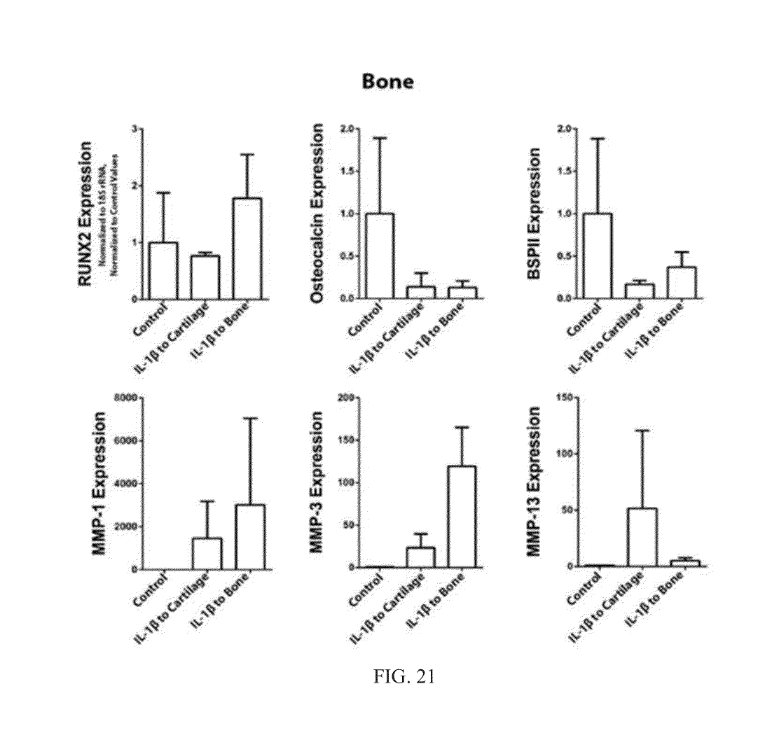

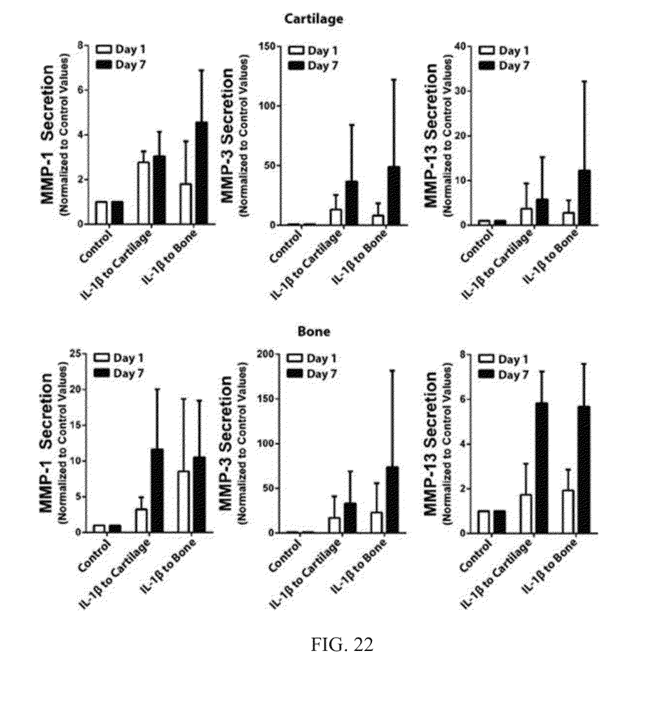

IL1β treatment on engineered osteochondral tissue. As described above, the microtissue bioreactor presented here, with its two separate medium flow systems and biphasic construct compartments, has the capability for targeted treatment of one (or both) tissue construct(s) with soluble factors. Osseous and chondral components were separately treated with the pro-inflammatory cytokine IL-1β (10 ng/ml) for 7 days (control conditions consisted of untreated osteochondral constructs), and the responses of each of the two components were separately analyzed. Media samples were collected from chondral and osseous components streams at day 1 and 7, and, after day 7, the osteochondral constructs were separated into osseous and chondral components, and each was separately analyzed for gene expression of catabolic genes (MMP-1, MMP-3, MMP-13) and either cartilage markers (Sox9, Col2, aggrecan) (FIG. 20) or bone markers (RUNX2, osteocalcin, BSPII) (FIG. 21). Media samples from the chondral and osseous components stream collected at days 1 and 7 were analyzed via MMP-1, MMP-3, and MMP-13 ELISAs (FIG. 22).

Treatment of chondral constructs with IL-1β caused decreases in expression of cartilage genes Sox9, Col2, and aggrecan, consistent with physiological outcomes of damaged or stressed cartilage. Chondral construct expression of these genes also decreased in response to IL-1β treatment of osseous constructs, suggesting signaling between the osseous and chondral components. Evidence of this osseous-to-chondral communication was even more apparent in results concerning expression of catabolic genes; expression of MMP-1, MMP-3, and MMP-13 of the chondral constructs increased substantially in response to IL-1β treatment of osseous component. Crosstalk between the two components was also detected in the case of chondral-to-osseous communication (FIG. 21). IL-1β treatment of the chondral construct caused decreases in expression of the bone genes osteocalcin and BSPII, and increases in MMPs production in the osseous construct, particularly MMP-13, which is one of the most important mediators of OA cartilage degradation.

ELISA analysis of MMPs secreted by the chondral and osseous components at different time points allowed for observations on the rate of signal propagation between the two components (FIG. 22). For example, the chondral construct responded to IL-1β treatment of the osseous component with increases in MMP-1, MMP-3 and MMP-13 secretion. The chondral MMP-13 response occurred quickly, within 1 day, while the chondral MMP-1 response took 7 days. The chondral MMP-3 response time was intermediate between those of MMP-13 and MMP-1. Again, these results are interesting considering the central role MMP-13 plays in cartilage degeneration. The osseous construct response to treatment of chondral component with IL-1β, on the other hand, was quick yet increased further over time, and by day 7 was overall stronger than the chondral responses to the osseous component treatment.

Gene expression and protein levels of MMPs were not be expected to be necessarily consistent due to differences in the ways in which ELISA and PCR samples were collected and measured. ELISA samples consisted of culture media conditioned by cells for 24 hours and were collected at the day 1 or day 7 for each experiment. The proteins contained in day 1 samples were secreted between days 0 and 1, and day 7 samples contained proteins secreted between days 6 and 7. PCR samples, on the other hand, were collected after 7 days of cultures and represent the expressional activities taking place at the moment of collection. In other words, the mRNA levels analyzed by PCR at day 7 are not necessarily totally reflective of the protein levels analyzed by ELISA in day 7 conditioned media samples. This disconnect between PCR and ELISA measurements may be more pronounced in MMPs, which need to be translated, secreted, and then diffuse out of the 3-D construct before they are detected by ELISA. Furthermore, differences between PCR and ELISA values also arise from differences in normalization. PCR results are normalized to 18S rRNA expression, thereby taking into account cell number. ELISA results are instead representation of the entire culture and normalized to control conditions. Thus, any experimental treatment that may affect cell number would have a larger impact on ELISA results than PCR results. Since chondrocytes are particularly sensitive to IL-1β, this may explain why results concerning cells of the chondral component exhibit the greatest degree of inconsistency between PCR and ELISA measurements when chondral constructs are directly stimulated by IL-1β.

### Discussion

This study describes the development of a novel bioreactor system for the engineering of osteochondral tissue. Real time RT-PCR and histological analyses showed that hBMSCs-derived naïve constructs have been successfully differentiated into cartilage-like tissue on the top and bone-like tissue on the bottom, using separated culture medium for 4 weeks. A transition layer between 2 tissues is also observed. The response of engineered osteochondral tissue to IL-1β treatment was further tested. The results show that IL-1β exposure decreases the ECM anabolic gene expression, but greatly enhances the levels of MMP expression and secreted amount into the medium. Interestingly, the IL-1β insulted osseous construct induces a catabolic gene expression response into the untreated chondral component which is not due to leakage of IL-1β, suggesting active osseous-chondral interaction and the likely importance of bone injury in OA development.

In this study, a dual-chamber bioreactor has been developed to generate and maintain osteochondral constructs derived from human hBMSCs. The design parameters included individual compartments to separate the chondral and osseous microenvironments that are individually accessible for the introduction of bioactive agents and/or candidate effector cells, tissue and medium sampling, and compositional assays, including non-invasive imaging techniques. Furthermore, the total dimension and geometry of the bioreactor matches that of a multi-well culture plate chamber for the development of medium- to high-throughput analysis. Validation of the system included successful, simultaneous differentiation of osseous and chondral constructs from hBMSCs from the same source (pooling of three donors) and subsequent application of IL-1β, a potent inflammatory mediator implicated in OA pathophysiology, to test the physiological response of the OCC.

In the course of 6 weeks, hBMSCs undergo tissue-specific differentiation in response to the tissue specific growth media and hydrogel composition provided. The differentiating hBMSCs expressed tissue-specific transcription factors and ECM molecules, as shown by RT-PCR and histological staining. Furthermore, there was an indication of a basophilic, tidemark-like zone separating the chondral and osseous components. This region is quite broad and therefore not an artifact. This shows a biochemically relevant, “anabolic” or “homeostatic” interaction between the chondral and osseous components. The recreation of the a tidemark-containing biphasic tissue is vital to drug testing using an osteochondral organotypic culture since changes in the OCJ are mechanistically involved in OA progression and likely to be a target of toxicants and DMOADs.

The response of the MSC-based osteochondral tissue to IL-1β was subsequently tested. The test served two purposes: (1) to validate the utility of the bioreactor in osteochondral studies and (2) to assess the physiological replication of the OC tissue by the MSCs in this bioreactor. Specifically, a detectable communication between the tissues was sought. IL-lbeta is almost ubiquitous in inflammatory diseases, is prominent in advanced OA in both the cartilage and synovial lining, and is frequently employed as a pathogenic initiator in in vitro models of OA. Application of IL-1β to both osseous and chondral components results in clear matrix degeneration and phenotypic changes in the resident cells, similar to what has been observed in monocultures of chondrocytes and osteoblasts. While chronic degeneration of the articular surface is most prevalent in OA, alterations in the subchondral bone or articular cartilage may be the primary trigger in OA. Using the bioreactor in this study, interactions between cartilage and bone were able to be studied which may contribute to OA progression

Clear osseous and chondral tissues interactions are observed when IL-1β is applied to the osseous component, which results in low levels of anabolic gene expression (50X9, Col2 and aggrecan) but robust expression of MMPs in the cartilage component. The MMP expression patterns are further validated by ELISAs. Conversely, application of IL-1β to the cartilage induces in bone low levels of anabolic bone gene expression (Runx2, OPN, and BSPII) but robust expression of MMPs. This apparently contradictory simultaneous induction of anabolic and catabolic processes within a tissue is entirely in keeping with the hypothesis that OA begins initially with a shift in the balance between anabolic and catabolic activities, followed by phenotypic changes in the cells in response to the modified environment. To some degree, inflammation can have beneficial effects on tissues, but at higher concentrations, inflammatory mediators induce tissue remodeling/destruction. Focusing on the response of chondral component to IL-1β treatment of the osseous component, it is interestingly that direct application of IL-1β to the chondral component has a less impressive catabolic response than indirect exposure via the osseous component. This result implies that the affected osteoblasts in the osseous component are producing bioactive factors, in addition to IL-1β, that are causing greater catabolic responses than IL-1β itself and vice-versa. Further, it shows that there is biochemically relevant, “degenerative” communication between the cartilage and the bone.

The formation of an OCJ between regions of engineered cartilage and bone is often reported, but generally is not analyzed beyond histological identification. In this study, opposing chondrogenic and osteogenic nutrient gradients were employed to stimulate OCJ formation by naïve, differentiating MSCs. It is thus difficult to relate the potential tidemark development in the described construct with frequently reported constructs that combine solid, porous polymeric sponges and hydrogels for osteochondral engineering in vitro or in vivo because of the great disparity in tissue architecture and scaffold biochemistry. Tidemark development similar to what is seen here has been reported in studies employing microparticle-mediated spatially-restricted growth factor release, microbead-encapsulated MSC-derived chondrocytes and osteocytes, MSCs encapsulated within scaffold material gradients, and MSCs stimulated by growth factor gradients. In all cases, the basophilic tidemark is indistinct and broad, particularly in models without loading. It is expected that appropriate mechanical loading and enhancement of cell differentiation, e.g., with an oxygen gradient may enhance the collection of metabolites to form the tidemark at the deep zone/calcified cartilage interface, particularly if differentiation is enhanced with an oxygen gradient and/or the addition of hydroxyapatite.

While formation of OCJ has been reported in in vivo implanted cell-seeded scaffold, there have been relatively few studies using a controlled bioreactor as reported here. The features of the current bioreactor design, including separate compartments for the “chondral” and “osseous” microenvironments supplied by independent tissue-specific media that can be controlled and regulated via introductions of bioactive agents or candidate effecter cells, and capability of individual sampling of the different compartments, are thus of potential value in allowing more individual manipulations. Specifically, its application for the assessment of drug and environmental factor toxicity is envisioned. Catabolic insults to one tissue component comprising the osteochondral unit may influence the other in a manner reminiscent of tissue degeneration in OA. Of particular interest is to investigate the communication of biomechanical signals/forces. This may be the first evidence of communication between different compartments of an osteochondral construct in response to catabolic cues (IL-1β). The tissue responses reported here reflect a subset of pathophysiological conditions reported in in vitro and in vivo models of OA. IL-1β is utilized in models of both rheumatoid and OA, with the effect of causing cartilage matrix breakdown and down regulation of cartilage matrix gene expression as shown here. In contrast, IL-1β has been reported to induce increased bone matrix deposition, although matrix of inferior quality, which may explain the response of the osseous component to direct exposure to IL-1β in the described model. The fact that catabolic gene expression in the osseous component is more enhanced by exposure of the overlying chondral component to IL-1β suggests that the chondral construct is producing additional signals and catabolic factors that travel to and affect the osseous component below, possibly constituting a form of inter-cellular communication not previously reported.

In summary, a new microfluidic based, multi-chamber bioreactor was fabricated for osteochondral differentiation and toxicity testing. Clear biphasic tissue differentiation was demonstrated in response to opposing chondrogenic and osteogenic gradients produced by tissue-specific differentiation factors supplied by independent medium streams to the chondral and osseous components of the construct. Also, MSC-based chondral and osseous tissues were shown to be capable of responding to IL-1β in a relevant manner, and changes in one tissue compartment are communicated, and perhaps amplified, to the other along the osteochondral axis. This bioreactor/organotypic osteochondral culture combination will enable a focus on the relationship of cartilage and bone in growth and degeneration and perhaps help to elucidate the roles of each tissue in OA. Finally, with minimal modification and appropriate coupling, the bioreactor described here can be adapted into a tissue component of an interacting multi-tissue bioreactor platform to study systemic tissue interactions.

### EXAMPLE 6

### Repair of Osteochondral Defect Using Photocrosslinked Gelatin/Hyaluronic Acid Scaffold

Osteoarthritis (OA) affects about 60% of men and 70% of women above 65 years of age, and represents a global growing health and economic problem. A visible light-activated methacrylated gelatin/hyaluronic acid (mGL/mHA) hydrogel, which is biocompatible and biodegradable, can maintain high cell viability and promote the encapsulated human mesenchymal stem cells (hMSCs) to undergo chondral or osseous differentiation with high efficiency by chondrogenic or osteogenic stimulation. The applicability of this novel scaffold for osteochondral repair in vivo was further examined using a rabbit model.

A cylindrical defect of 4 mm in diameter and 3 mm in depth was surgically created in the young (5-7 weeks) or aging (13-15 weeks) rabbit femoral condyle and a 3D predifferentiated hMSCs -biomaterial construct was fabricated in situ to fully fill the lesion site. The efficacy of the in situ formed neo-cartilage to repair the lesion site was estimated at 12 weeks post implantation.

Young rabbits showed strong self-healing ability 12 weeks post-surgery. In all groups including defect without treatment, the osteochondral lesion was completely covered. The regeneration variation was significant among aging rabbits. Based on the macro appearances of defects, the application of scaffold generally improved filling of the defect and osteochondral regeneration in aged rabbits. Further assessment was performed by microCT and histological analysis.

Results from this in vivo study strongly suggest the feasibility of using this injectable mGL/mHA scaffold produced by means of visible light-mediated crosslinking for clinical repair of osteochondral defects.

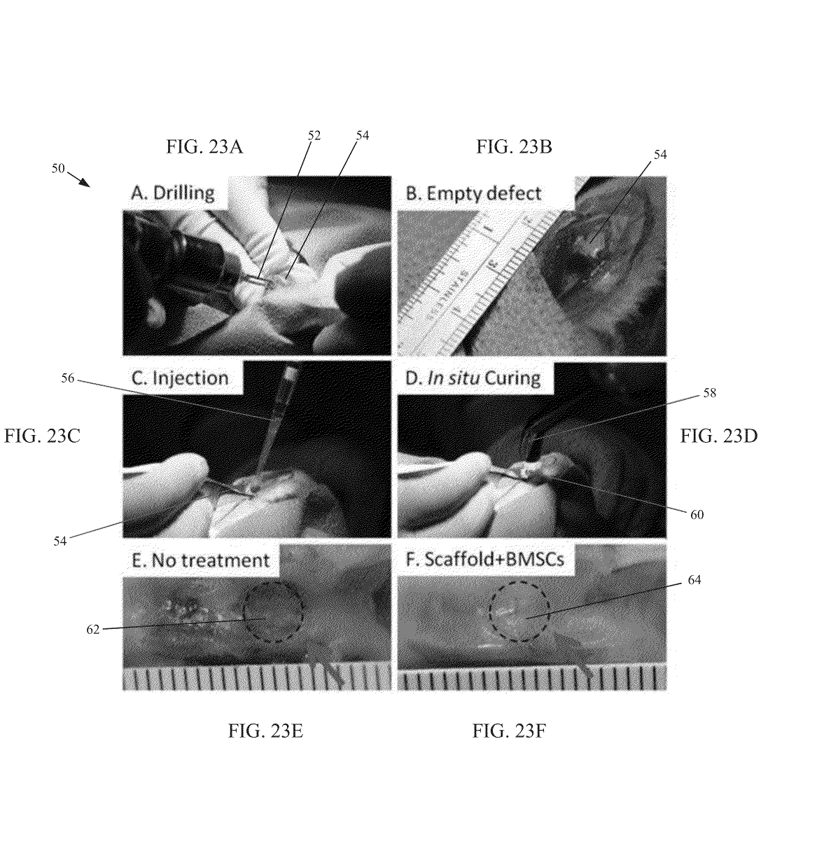

FIGS. 23A-F illustrate an exemplary method 50 for repairing a cartilage defect using an injectable photocrosslinking gelatin/hyaluronic acid scaffold. FIG. 23A shows drilling with a tool 52 and removal of unwanted material at the defect site 54. FIG. 23B shows the empty defect 54 after drilling. FIG. 23C shows the injection of the scaffold containing BMSCs from an injection device 56. FIG. 23D shows in situ curing of the injected material 60 using visible light from a light source 58. FIGS. 23E and 23F compare results using the illustrated treatment versus results without the treatment. After 12 weeks, a significant improvement in tissue repair 64 is evident in FIG. 23F compared to the tissue 62 with no treatment as shown in FIG. 23E.

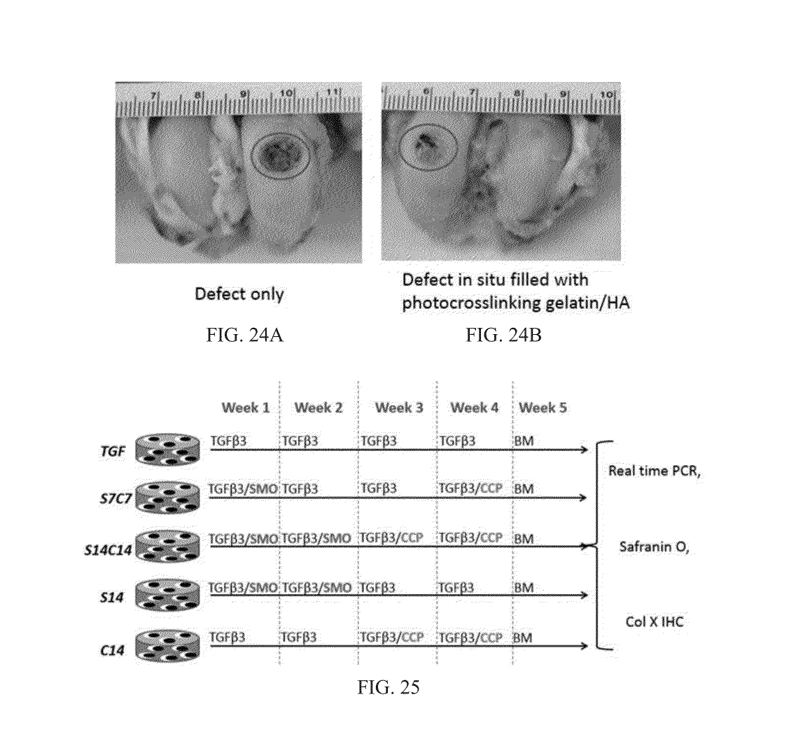

The applicability of the injectable, photocrosslinkable gelatin/hyaluronic acid scaffold for a large animal defect was tested in a goat knee cartilage defect model, and is illustrated in FIGS. 24A and 24B. FIG. 24A shows a defect seven days after surgery without the use of the injectable gelatin/hyaluronic acid scaffold, and FIG. 24B shows a defect seven days after surgery with the use of the injectable gelatin/hyaluronic acid scaffold. An observable improvement in defect repair is evident with the introduction of the MSC-containing hydrogel.

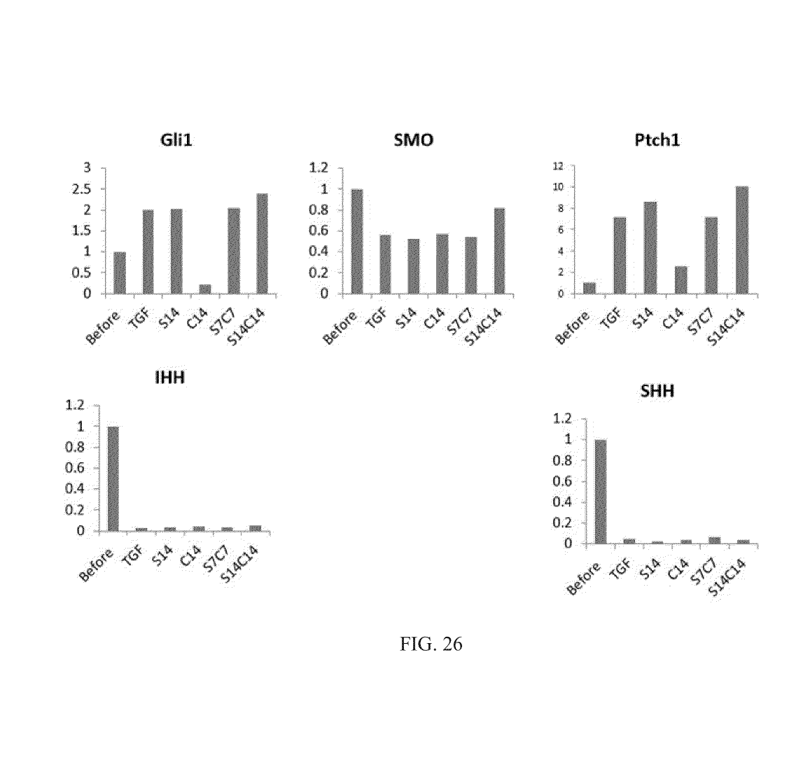

FIGS. 25-27 below illustrate exemplary tests and results regarding how engineered cartilage using hBMSCs encapsulated in a photocrosslinked hydrogel poly(DL-lactic acid)(PDLLA)-polyethylene glycol (PEG) (PDLLA-PEG) scaffold may be used to study the functional consequences of perturbing Hedgehog signaling on cartilage bioactivity and health. Two agents were used: a Smo Agonist (SMO) and an antagonist, cyclopamine (CCP). FIG. 25 shows the testing parameters used in testing the effects of applying various drugs to engineered cartilage tissue (chondrogenesis induced with TGFβ3). FIGS. 26 and 27 show the results of these tests.

Take together, results from this study clearly illustrate that the hCEMTs, composed with chondrogenic hBMSCs encapsulated in photocrosslinked hydrogels, recapitulate the biological activities of native cartilage.

Furthermore, a visible light-activated methacrylated gelatin/hyaluronic acid (mGL/mHA) combination hydrogel has been created, which is biocompatible and biodegradable, maintaining high cell viability and promoting the encapsulated human mesenchymal stem cells (hMSCs) to undergo chondral or osseous differentiation with high efficiency by chondrogenic or osteogenic stimulation. The applicability of this novel biodegradable scaffold for osteochondral repair was further examined using a rabbit model.

In an exemplary experiment, a cylindrical defect of 4 mm in diameter and 3 mm in depth were surgically created in the young (5-7 weeks) or aging (13-15 weeks) rabbit femoral condyle and a 3-dimensional (3D) predifferentiated hMSC-biomaterial construct was fabricated in situ to fully fill the lesion site. The efficacy of the in situ formed neo-cartilage to repair the lesion site was estimated at 12 weeks post implantation. Young rabbits showed strong self healing ability 12 weeks post surgery. In all groups including defect without treatment, the osteochondral lesion appeared completely covered. Tissue regeneration variation was significant among aging rabbits. Based on the macro appearances of defects, the application of scaffold generally improved filling of the defect and osteochondral regeneration in aged rabbits. Further assessment was performed by microCT and histological analysis. Results from this in vivo study illustrate that the injectable, visible light crosslinked mGL/mHA combination scaffold can be used for clinical application in the repair of osteochondral defects.

### EXAMPLE 7

### BMP-2 Gene & Cell-Functionalized 3D Scaffolds for the Repair of Cranial Bone Defect

Although bone is one of the most commonly repaired tissues of the body, fracture-delayed unions or non-unions constitute 10-15% of the approximately 6.5 million bone fractures reported in the US yearly. Autologous bone grafts have been considered as the clinical “gold standard”, but this method causes donor site morbidity and is greatly restricted by tissue availability. Tissue engineering using adult mesenchymal stem cells (MSCs) seeded within biomaterial scaffolds has shown the potential to enhance bone healing. An injectable, visible light-based photocrosslinking (VL-PCL) method to fabricate a 3D hydrogel, as described herein, is especially effective in producing scaffolds with defect-matching architecture and may be uniformly seeded with high viable stem cells and viral vectors, and can allow custom-designed bone tissue engineering and promote construct placement and integration into bone lesion sites.

In addition to scaffolds, another major requirement in some stem cell-based bone tissue engineering methods is the promotion of robust osteogenic differentiation of cells, which requires sustained stimulation with soluble osteoinductive biofactors, such as bone morphogenetic protein-2 (BMP-2). However, the direct application of these factors remains challenging due to their short half-life and rapid systemic clearance into the bloodstream. Given the well demonstrated benefits of recombinant adeno-associated viral (rAAV) vector, including long-term gene transfer efficiency and relative safety, a combination of gene and cell therapies can be been applied to support clinical bone tissue regeneration. However, traditional ex vivo gene transfer to stem cells, prior to their seeding onto engineered biomaterial scaffolds, is a time consuming process and requires multiple steps of cell culture in vitro.

In this study, the applicability of single-step VL-PCL fabrication of 3D gelatin scaffold was critically assessed for the delivery of rAAV-viral vector encoding BMP-2 to address the need for sustained BMP-2 presence within scaffolds for the repair of mouse cranial bone defect. In this method, rAAV-BMP-2 and human bone marrow MSCs (hBMSCs) are simultaneously included into poly(DL-lactic acid)(PDLLA)-polyethylene glycol (PEG) co-polymer PDLLA-PEG scaffolds during scaffold formation using visible light. The subsequent release of rAAV-BMP-2 constructs from the scaffold matrix can result in efficient expression of BMP-2 by hBMSCs seeded within the scaffolds, and can thus enhance their osteogenic differentiation and the formation of new bone upon implantation in the cranial defect in mice or other bone defect.

The rAAV6-green fluorescent protein (GFP) and rAAV6-BMP-2 vectors used this study were designed and prepared using an established protocol. Methacrylated gelatin (mGL) was produced using existing lab protocol. hBMSCs were obtained from total joint arthroplasty patient. Live cell VL-PCL was performed using a published protocol using 10% mGL and 10×10⁶ hBMSCs/ml. To address potential safety concerns in future clinical applications, the cytocompatibility of gene-activated scaffold was tested by determining hBMSC viability within scaffold using Live/Dead staining. Cell metabolic activity was also analyzed using the MTS assay. Transfection efficiency was determined by counting GFP-positive cells ratio. Animal study included the following 4 groups (7 mice/group): (1) scaffolds loaded with rAAV6-BMP-2 and hBMSCs; (2) scaffold loaded with BMP-2 protein (1 μg/ml) and hBMSCs; (3) scaffolds loaded with hBMSCs that were previously transduced ex vivo with rAAV6-BMP-2; (4) defect only. A 0.5 cm diameter critical size of cranial bone defect model was generated by trephine after mouse was anesthetized, and then the constructs from groups described above were filled with in situ-fabricated biocompatible biomaterial in cranial bone defects. The mice were anesthetized and micro-CT imaging was used to evaluate the skull bone healing process, including analysis of bone volume and density, up to 6 weeks.

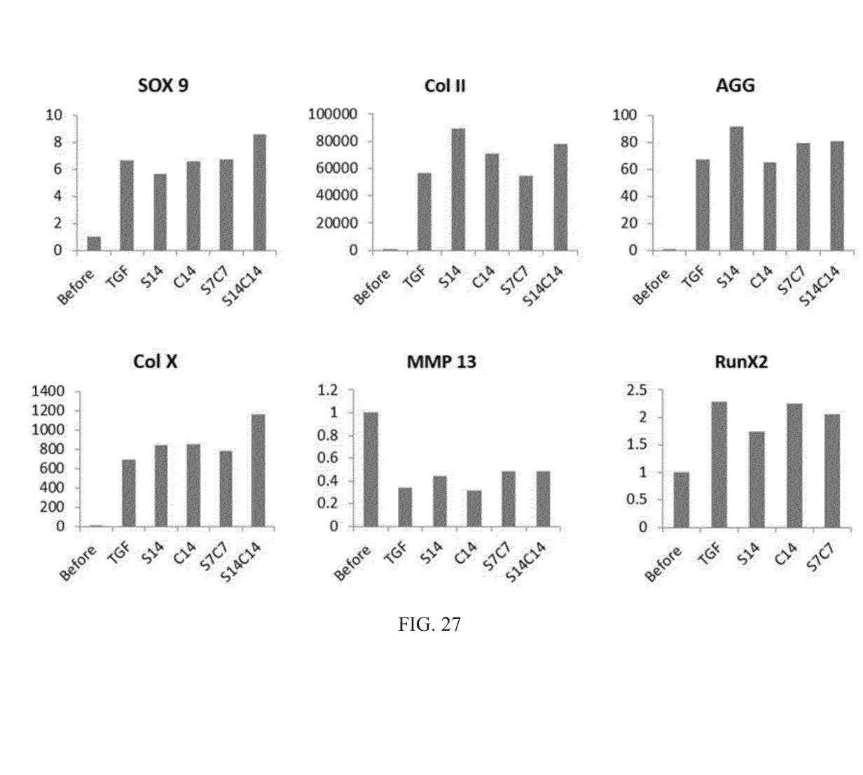

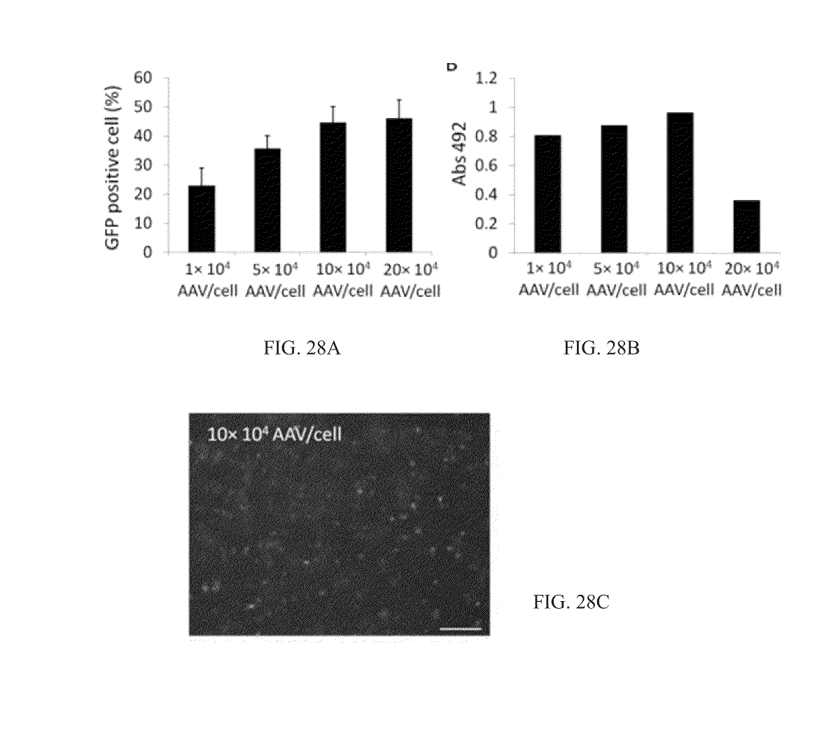

As shown in FIGS. 28A-28C, both rAAV and hBMSCs survive during the scaffold fabrication process. Transfection efficiency increased with increased rAAV/hBMSCs ratio, but cell viability decreased at 20×10⁴ AAV/cell. 10×10⁴ AAV/cell is thus a desirable ratio in maintaining cell viability and achieving transduction efficiency.

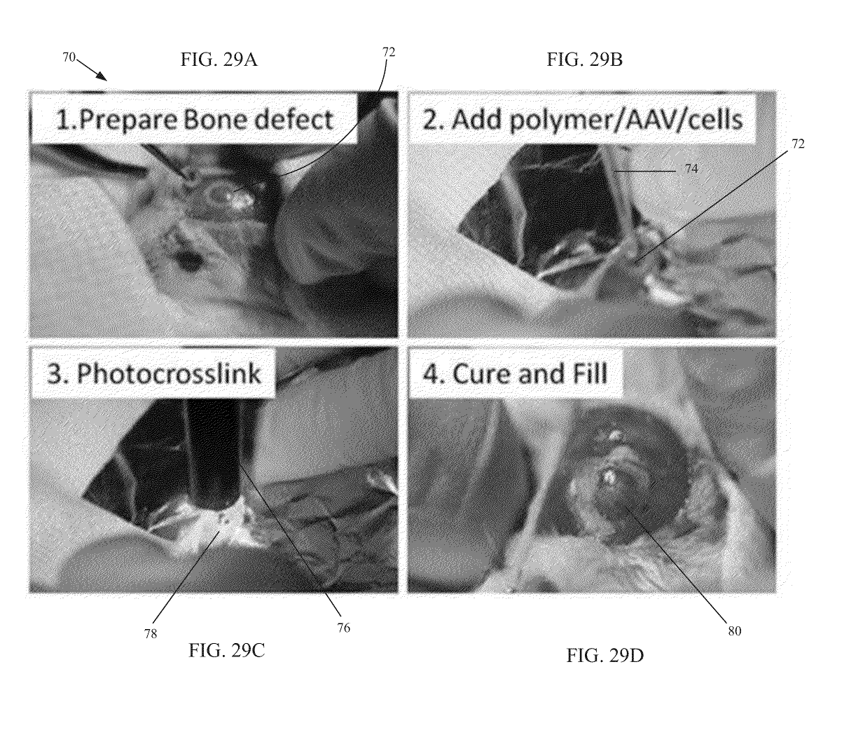

This novel construct was then applied for cranial bone defect repair in SCID mice, as shown in FIGS. 29A-29D. FIG. 29A shows the prepared bone defect site 72. FIG. 29B shows the addition of injected materials into the site 72 using an injection tool 74. FIG. 29C shows photocrosslinking of the injected materials 78 using a light source 76. FIG. 29D shows the site filled with cured materials 80.

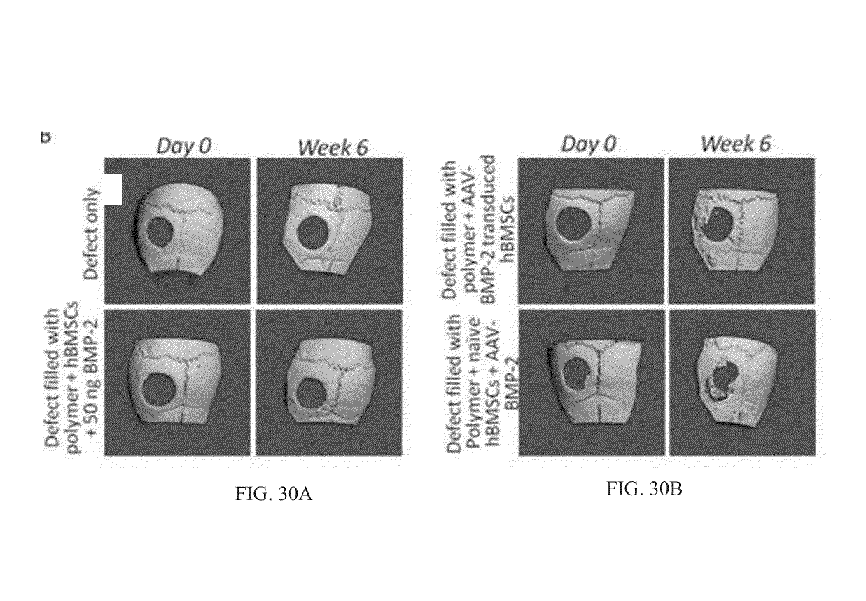

FIGS. 30A and 30B show preliminary micro-CT imaging results illustrating that rAAV BMP-2 & hBMSCs-activated PEG-PDLLA scaffolds, with single step fabrication, effectively promoted bone regeneration in mice cranial bone defects, in a manner significantly superior to that of scaffolds loaded with hBMSCs/BMP-2 protein, and were remarkably compatible to scaffolds loaded with hBMSCs previously transduced ex vivo by rAAV-BMP-2.

Repair of large bone defect remains a challenge in modern trauma and orthopedic medicine. In this study, a single-step fabrication of bioactive bone scaffolds was developed by simultaneously packing rAAV-BMP-2 and BMSCs. Both rAAV and cells survive during the fabrication process, and successful transduction of rAAV into hBMSCs was also observed (optimized ratio: 10×10⁴ rAAV/cell). Using an in situ fabrication procedure, this novel bioactive construct was grafted into the cranial defect in mice and resulted in micro-CT detectable new bone formation. This novel gene and cell activated bone scaffold and analogous methods may also be used for the repair of bone defects in large mammals.

The results shown here demonstrate the feasibility of combining gene and cell therapy with biomaterial engineering via a single-step fabrication to generate a BMP-activated hBMSC-based construct for cranial bone regeneration. The efficiency of this procedure, which requires only less than one day from hBMSC harvesting to implantation, is highly desirable for clinical applications.

### EXAMPLE 8

### PSL Fabrication of Human Adipose Stem Cell-incorporated Biodegradable Scaffolds for Cartilage Tissue Engineering

The high incidence of osteoarthritis (OA) in the United States and very limited self-healing ability of cartilage present a need for the development of methods for cartilage regeneration. Scaffold construction with live stem cell incorporation and subsequent differentiation can be used to obtain tissues that mimic native cartilage. A particular demand of cartilage tissue engineering is the production of scaffolds that can meet the mechanical properties of the weight-bearing tissue. Projection stereolithography (PSL), in particular, offers high resolution and processing speed as well as the ability to fabricate scaffolds that precisely fit the anatomy of cartilage defects using medical imaging as the design template. Accordingly, a visible-light based PSL (VL-PSL) system can be used to encapsulate human adipose-derived stem cells (hASCs) into a biodegradable polymer (poly-D,L-lactic acid/polyethylene glycol/poly-D,L-lactic acid (PDLLA-PEG))/hyaluronic acid (HA) matrix to produce live cell constructs with customized architectures. In an exemplary method, after fabrication, hASCs showed high viability (84%) and were uniformly distributed throughout the constructs, which possessed high mechanical property with a compressive modulus of 780 kPa. The hASC-seeded constructs were then cultured in Control or TGF-β3-containing chondrogenic medium for up to 28 days. In chondrogenic medium treated group (TGF-β3 group), hASCs maintained viability, proliferated, expressed chondrogenic genes and produced a collagen type II and glycosaminoglycan (GAG)-rich extracellular matrix, revealed by immunohistochemistry, Alcian blue and Safranin O staining, suggesting robust chondrogenesis within the scaffold. Without chondroinductive addition (Control group), cell viability decreased with time and showed poor cartilage matrix deposition. The PSL- and PLLA-PEG/HA based fabrication method, using adult stem cells, can therefore be used to produce mechanically competent engineered cartilage for joint cartilage resurfacing in OA patients.

### Introduction

Cartilage damaged by trauma, disease or aging demonstrates very limited capabilities for self-regeneration and ultimately results in osteoarthritis (OA). Given the high prevalence of osteoarthritis in the United States (27 million affected), which is projected to increase due to population aging as well as the obesity epidemic, methods toward managing and treating these cartilage defects are critical. While there exist procedures to treat these defects, such as microfracture and osteochondral grafting, they either finally lead to the formation of fibrocartilage or are limited by tissue availability. In addition, allografting bone and cartilage has the potential risk of infection and disease transmission. Severe cartilage defects ultimately require total joint arthroplasty to reduce pain and improve mobility, but it involves a major surgery and ends the biological life of cartilage. As such, a regenerative approach that can restore the native properties of cartilage represents an attractive alternative.

Recently, regenerative medicine has garnered high interest, which involves the development of cartilage-like constructs through the use of cells, growth factors, scaffolds and combinations. For example, autologous chondrocyte implantation (ACI) and matrix-induced ACI (MACI) are popular procedures that harvest and in vitro expand chondrocytes from the patient's own tissue which are then grafted into the cartilage defect site with or without accompanying extracellular matrix. However, this source of healthy chondrocytes is limited and requires several weeks of cell culturing to obtain adequate cell numbers for transplantation, which results in chondrocyte dedifferentiation. Thus, adult tissue-derived stem/progenitor cells, such as mesenchymal stem cells (MSCs), that have been shown to have the ability to differentiate into a variety of cell lines including chondrocytes, offer a promising substitute for the primary chondrocytes. In particular, adipose tissue derived MSCs (ASCs) have attracted recent attention because they are isolated in higher quantities than stem cells found from other sources, such as bone marrow, and obtainable through minimally invasive procedures, thus offering the advantage in reducing or even eliminating in vitro expansion to allow point-of-care application. In addition, ASCs have been shown to be beneficial in cartilage healing, including reducing pain, and improving function for aging patients with knee OA after intra-articular injection.

A key component in cartilage tissue engineering is a biomaterial scaffold to deliver the candidate cells, such as MSCs, to the defect site and to also temporarily fill the defect to facilitate cell growth. In addition to biocompatibility, the ideal scaffold for cartilage tissue engineering should possess viscoelastic hydrogel-like characteristics that mimic the mechanical properties and functions of native cartilage. Optimally, the biomaterial will also be biodegradable such that as the stem cells differentiate into chondrocytes and produce a cartilaginous extracellular matrix (ECM), the scaffold degrades and the newly secreted ECM remodels the construct into a cartilage-like tissue. In addition, precise fitting of the scaffold into the local structural geometry of the defect and the host tissue anatomy is critical for enhancing the repair process, such that the absence of gaps will optimize integration between implants and native tissue and allow continuous load distribution. To date, many technologies have been developed in the fabrication of scaffolds with different geometry and internal architecture. Traditional technologies such as solvent casting, particulate leaching and electrospinning do allow for limited control of structure, but they are not able to perform the fabrication of the highly detailed structures in a patient by patient basis. In addition, these methods require the use of either organic solvents or conditions unfavorable for cell survival, thus limiting their ability to seed cells directly within scaffolds.

Solid free-form fabrication (SFF) methods offer the ability to control both the macrostructure as well as microstructure of the scaffolds. With the utilization of medical imaging and computer-aided design (CAD) model guided scaffold fabrication, SFF methods can create scaffolds with precise architectures. Different SFF methods can be applied in the fabrication of a variety of biomaterials, including laser sintering, stereolithography, fused deposition modeling, and 3D printing. Of these techniques, stereolithography is the most accurate and is based on light-induced photo-polymerization of derivatized monomers. Projection stereolithography (PSL) in particular is a method that is of great interest due to its high fabrication rate and resolution. By utilizing a layer-by-layer based image projection of defined thickness, fabrication times are drastically reduced from conventional stereolithography. As described herein, PSL using visible light illumination (VL-PSL) can be applied in a one-step live cell-scaffold fabrication in which highly viable human ASCs (hASCs) are uniformly incorporated within the polyethylene glycol diacrylate (PEGDA) scaffolds. While this procedure for the first time allows live cell-scaffold fabrication using VL-PSL, it can be limited by the monomer that is used because PEG is not biodegradable and does not provide cell-binding ligands. In addition, Percoll may be used to suspend the cells during the fabrication, which introduces additional non-native molecules into the final construct. The extent to which chondrogenesis of hASCs is supported in the PEG scaffold can also be of concern.

A number of properties are desirable for a biomaterial suitable for implantation for cartilage repair, such as biodegradability, biocompatibility, strong compressive modulus, and/or presence of cell binding ligands. It is desirable that the material also be water-soluble in order to not interfere with cell survival during the photo-crosslinking scaffold fabrication process. A novel hybrid matrix is described herein in which a synthetic polymer, poly-D,L-lactic acid/polyethylene glycol/poly-D,L-lactic acid (PDLLA-PEG), serves as the structural component, and hyaluronic acid (HA) as the co-polymer to supply cell binding ligands and to inhibit cell settlement during PSL fabrication owing to its viscous property. HA is a glycosaminoglycan present in abundance in the cartilage ECM and synovial fluid and has been shown to promote hASC chondrogenesis through interaction with its surface receptor, CD44.

In an exemplary study described below, hASCs were suspended in a methacrylatd PDLLA-PEG and HA (mPDLLA-PEG) solution and subjected to VL-PSL with different CAD architectures. The cell-seeded fabricated scaffolds were cultured in control medium or TGF-β3-containing chondrogenic medium for up to 4 weeks. Cell viability was examined at different time points and the progression of chondrogenesis of hASCs within the scaffolds was assessed by mechanical testing, real time reverse transcription polymerase chain reaction (RT-PCR) analysis of gene expression, and histological staining. The results showed that the VL-PSL produced hybrid scaffolds precisely mimicked the CAD structure and maintained high cell viability during the fabrication process. In addition, mPDLLA-PEG/HA scaffolds supported efficient hASC chondrogenesis upon induction. Accordingly, the methods described in this example can be used for the development of personalized stem cell-based repair of articular cartilage defects.

### Human Adipose Stem Cell Isolation

hASCs were isolated from lipoaspirate using an automated cell isolation system. The isolated cell pellets were re-suspended in expansion medium (EM: DMEM-high glucose, 10% MSC-certified fetal bovine serum (FBS), 100 units/ml penicillin, 100 μg/ml streptomycin) and plated on tissue culture flask. After 3 days, the nonattached cells were washed out with Hank's Balanced Salt Solution (HBSS). The medium was changed every 3 days. At 80% confluence, cells were detached with 0.25% trypsin in 1 mM EDTA and passaged. All experiments were performed with hASCs obtained at passage 3 (P3). hASCs used in this study were pooled from 2 patients (36 and 28-year females).

### Synthesis of Methacrylated PDLLA-PEG (mPDLLA-PEG) and HA (mHA)

To prepare the mPDLLA-PEG, 50 g of PEG (4 kD molecular weight) was placed into a 250 mL flask and subjected to 600 W microwave irradiation for 3 minutes. Subsequently, 3.5 g (2.80 ml) of stannous octoate [Sn(Oct)2] was added to the molten PEG followed by addition of 7.2 g poly-D,L-lactide. The mixture was subjected to 600 W microwave irradiation for 1 minute. The initial PDLLA-PEG polymer was precipitated in 500 ml cold isopropanol, and was dried under vacuum for 2 days. Dry polymer was dissolved in 100 ml dichloromethane (DCM), followed by addition of 3 equivalents of tetraethylammonium chloride (TEA, ˜5.25 ml) and 3 equivalents of methacrylic anhydride (MA, ˜5.60 ml). The reaction mixture was covered with punctured Parafilm and allowed to stir at room temperature for 7 days. After completion of the reaction, the mixture was precipitated into diethyl ether. For further purification, the macromer was redissolved in minimal amounts of chloroform and reprecipitated in diethyl ether. mHA was prepared by reacting methacrylic anhydride (MA) with sodium hyaluronate.

### Synthesis of Photoinitiator LAP

The visible light sensitive initiator lithium phenyl-2,4,6-trimethylbenzoylphosphinate (LAP) was synthesized as described by Fairbanks et al. (Fairbanks et al., 2009)

### PSL Fabrication of Live Cell Constructs

The PSL apparatus was equipped with digital light processing (DLP) technology. Visible light mode (Hg illumination utilizing a UV barrier filter) was used, with the curing depth of each layer set at 50 μm.

Solutions of polymer, LAP and Phenol Red dye were prepared in 50 ml tubes. For instance, the preparation of mPDLLA-PEG (30% w/v), mHA (0.5% w/v), LAP (0.6% w/v), and Phenol Red (0.025% w/v) was carried out as follows: polymer (12 g mPDLLA-PEG and 0.2 g mHA) was placed in the 50 ml tube followed by slow addition of HBSS close to the 40 ml mark and subsequent addition of LAP (240 mg) and Phenol Red (10 mg). The solution was titrated to pH 7.4 with 10 N NaOH and adjusted to 40 ml using HBSS.

P3 hASCs were pelleted by centrifugation and the supernatant was completely removed. The polymer solution prepared above was used to re-suspend hASCs at 4×10⁶cells/ml, and then the cell-polymer solution was immediately poured into the basement plate of the PSL device for printing with different 3D models as the template.

The fabricated constructs were detached from the platform and washed 3 times to remove uncured polymer solution. The constructs were cultured in control medium (CM, DMEM with 1% L-alanyl-L-glutamine, 55 μM sodium pyruvate, 1× antibiotic-antimycotic, and 1% Insulin-Transferrin-Selenium (ITS)) or chondrogenic medium (CGM, CM supplemented with 10 ng/ml transforming growth factor-β3 (TGF-β3), 100 nM dexamethasone, 50 μM L-ascorbic acid 2-phosphate, and 23 μM L-proline) up to 28 days, which were designated as Control or TGF-β3 group, respectively.

### Degradation Test

Fabricated scaffolds (5 mm diameter and 2 mm height) produced as described above but without cells were immersed in 5 ml HBSS and maintained in cell culture incubator at 37° C. HBSS was changed every 3 days. The degradation of polymer was estimated by measuring the mechanical property of scaffolds at different times.

Mechanical testing of scaffolds was conducted with a mechanical tester (Bose Electroforce model 3230 Series II). Briefly, the cylindrical scaffolds were placed between the compressive motor and load cell and subjected to 10% compression (0.2 mm) at 0.01 mm/sec. The stress-strain curve was then plotted and the linear area was used to calculate the compressive modulus of scaffolds.

### Live/Dead staining

At various time points post-fabrication, cell viability was assessed with the Live/Dead Viability/Cytotoxicity kit as examined by epifluorescence microscopy. The percentage of live cells was calculated as the number of green-staining cells divided by the total number of cells (green & red staining cells).

### Analysis of Gene Expression by Real-Time RT-PCR

Total RNA of the cells within the constructs was isolated using TRIZOL reagent and purified. Reverse transcription reactions were performed. Real-time PCR was performed with a thermocycler. All sample values were normalized to 18S rRNA using the 2^{-ΔΔCt} method.

### Hydroxyproline Quantitation

Total collagen content deposited within the constructs was determined by measuring hydroxylproline levels. The constructs (5 mm diameter and 2 mm thickness) were homogenized in water by grinding and hydrolyzed using the same volume of 12N HCl at 120° C. for 3 hours. Hydroxyproline content in constructs was quantitated using a Hydroxyproline Colorimetric Assay Kit.

### Histology

After 28 days culture, constructs were removed from the incubator, washed twice, and fixed in buffered paraformaldehyde (4%) for 1 day at room temperature. After washed with PBS for 3 times, they were then cryosectioned using a cryostat at 8 μm thickness. For assessment of glycosaminoglycan deposition, the slides were stained with either Alcian Blue or Safranin-O/Fast Green following standard protocols.

### Immunohistochemistry (IHC)

Enzymatic antigen retrieval was performed using chondroitinase/hyaluronidase (1 mg/ml and 5 mg/ml) at 37° C. for 30 min, and was suppressed with 1% horse serum in PBS for 45 min. After blocking endogenous peroxidase (3% H₂O₂ in methanol for 10 min) and nonspecific binding (1% horse serum), slices were incubated with primary antibodies against collage type II overnight at 4° C. After washing, biotinylated secondary antibodies were applied for 30 min. Staining was developed by treating samples with horseradish peroxidase (HRP)-conjugated streptavidin/NovaRED™ peroxidase substrate.

### Statistical Analysis

All studies were performed with 3 experimental replicates. Results were expressed as the mean±SD. Significant differences between control and chondrogenic groups were determined by one-tailed Student's t-test. Significance was considered at p<0.05(*) and p<0.01(**).

### Results

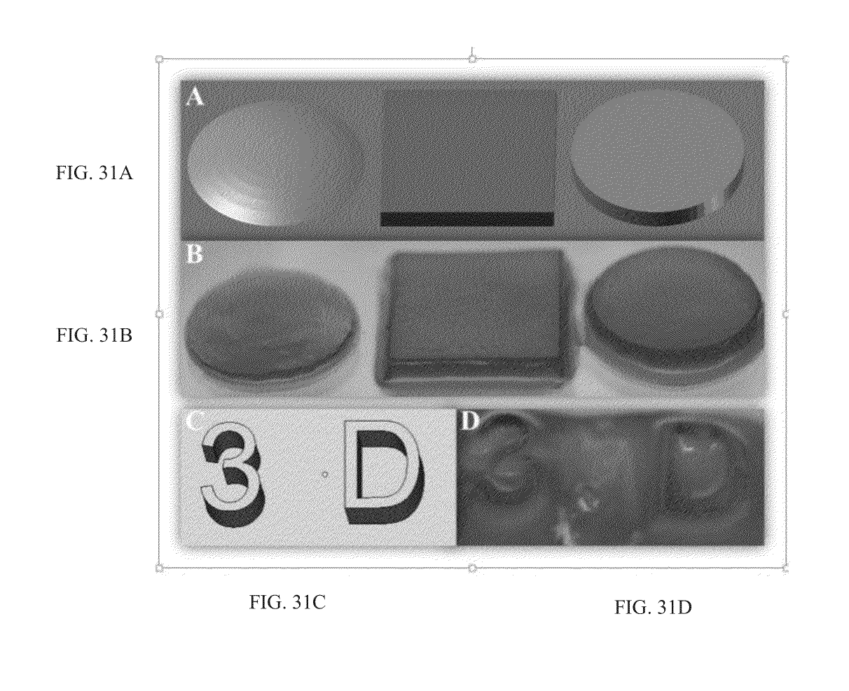

Constructs generated using VL-PSL were first assessed for their ability to faithfully replicate designed architectures. FIGS. 31A-31D illustrate the construction of various 3D shapes, including conical, cubic, and cylindrical (FIGS. 31A and 31B), and more complicated alpha numeric structures (FIGS. 31C and 31D). In all cases, the structures produced using VL-PSL mimicked the designs with high fidelity upon visual inspection.

### Degradation analysis

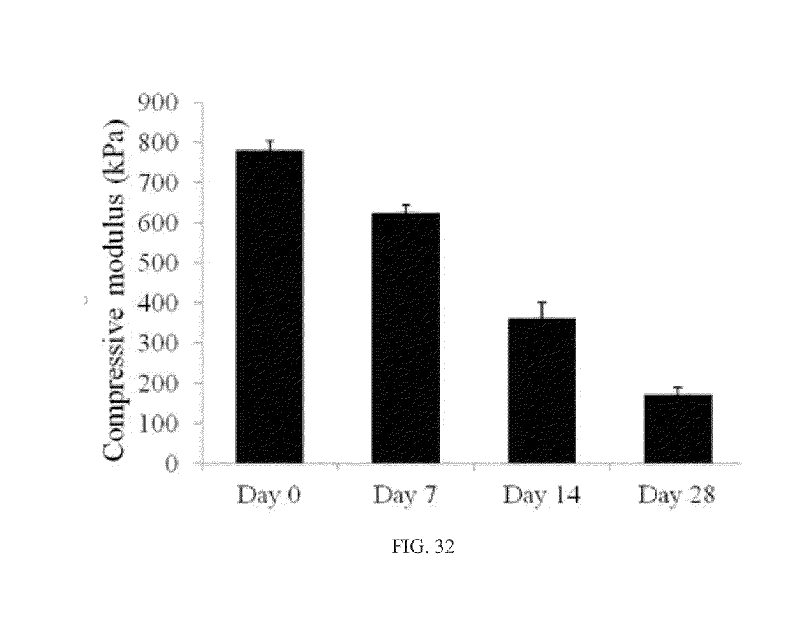

Due to the presence of ester bonds in the mPDLLA-PEG/HA co-polymers, they are expected to be degraded through hydrolytic cleavage. To test the degradation behavior, PSL-fabricated mPDLLA-PEG/HA scaffolds were incubated in PBS at 37° C. and their mechanical properties tested at different time points up to 4 weeks. As shown in FIG. 32, the compressive modulus of scaffolds significantly decreased with time, demonstrating structural degradation in aqueous solution. After 4 weeks, scaffolds retained only ˜25% of their original mechanical strength.

### Cell Viability Assessment

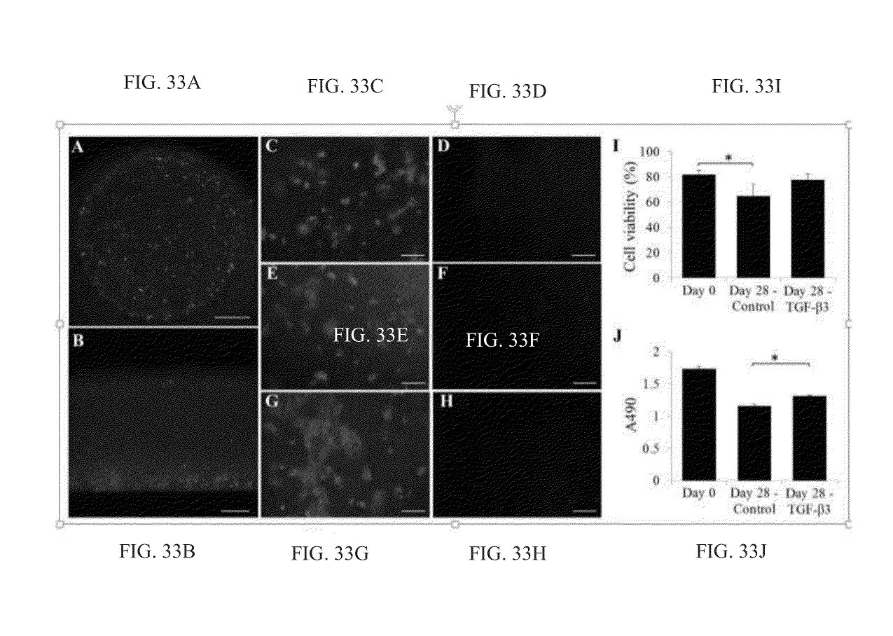

Cell viability was determined immediately after fabrication and also at 28 days after culturing in control and chondrogenic medium. FIG. 33A shows a surface view of a Calcein-AM stained construct, and FIG. 33B shows a cross sectional view of the construct at day 0. Uniform distribution of single cells throughout the construct was clearly seen, showing that cells remained suspended and separated from each other in the fabrication solution for at least 30 min. Cell viability was determined to be high at 81% after fabrication (FIGS. 33C, 33D, and 33I). After 28 days of culture in control medium, cell viability decreased to 65% (FIGS. 33E, 33F, 33I, and 33J). In contrast, chondrogenic medium supplemented with TGF-β3 not only maintained higher cell viability (77%, FIGS. 33G, 33H, and 33I) but also promoted cell proliferation as indicated by MTS assay (FIG. 33J). There is a statistically significant difference between the control and the TGF-β3 group in cell number.

### Chondrogenesis Analysis

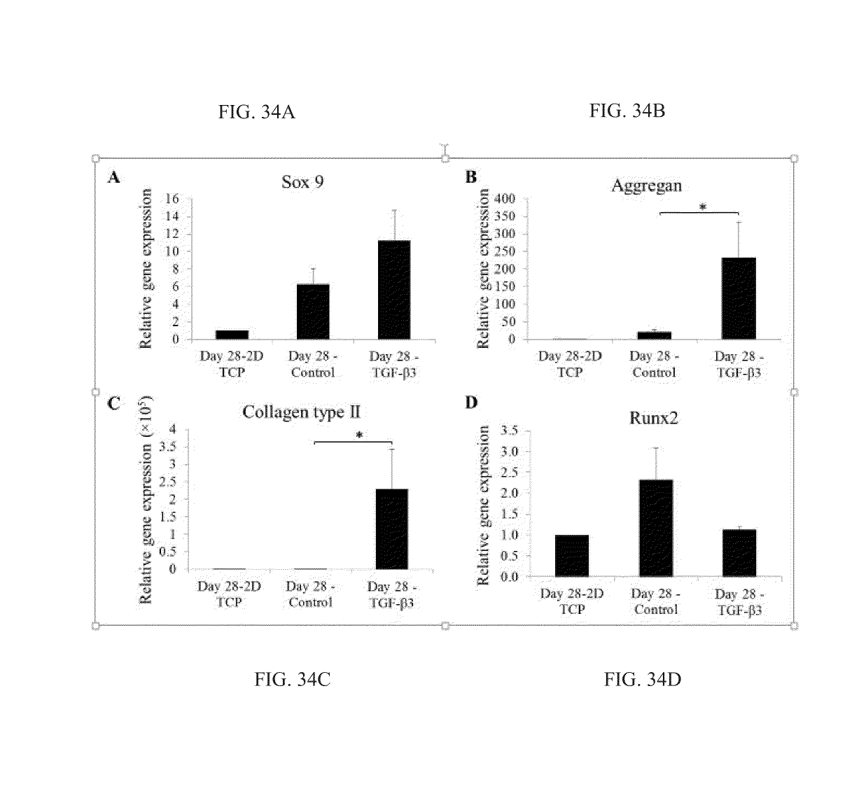

Chondrogenic differentiation of the hASCs seeded within the dense mPDLLA-PEG/HA scaffolds and exposed to TGF-β3 containing chondrogenic medium was analyzed by real time RT-PCR for the expression of genes associated with chondrogenesis. FIGS. 34A-34D show the relative levels of gene expression for Sox 9, aggrecan, collagen type II, and Runx2. All three chondrogenic genes, Sox 9, aggrecan and collagen II, were also significantly higher in the TGF-β3 group. In contrast, Runx2, an osteogenesis marker, expression was found to be higher in the control group. Thus, TGF-β3 chondrogenic medium effectively induced chondrogenesis and concurrently inhibited osteogenesis in hASCs encapsulated within the PSL-fabricated scaffolds.

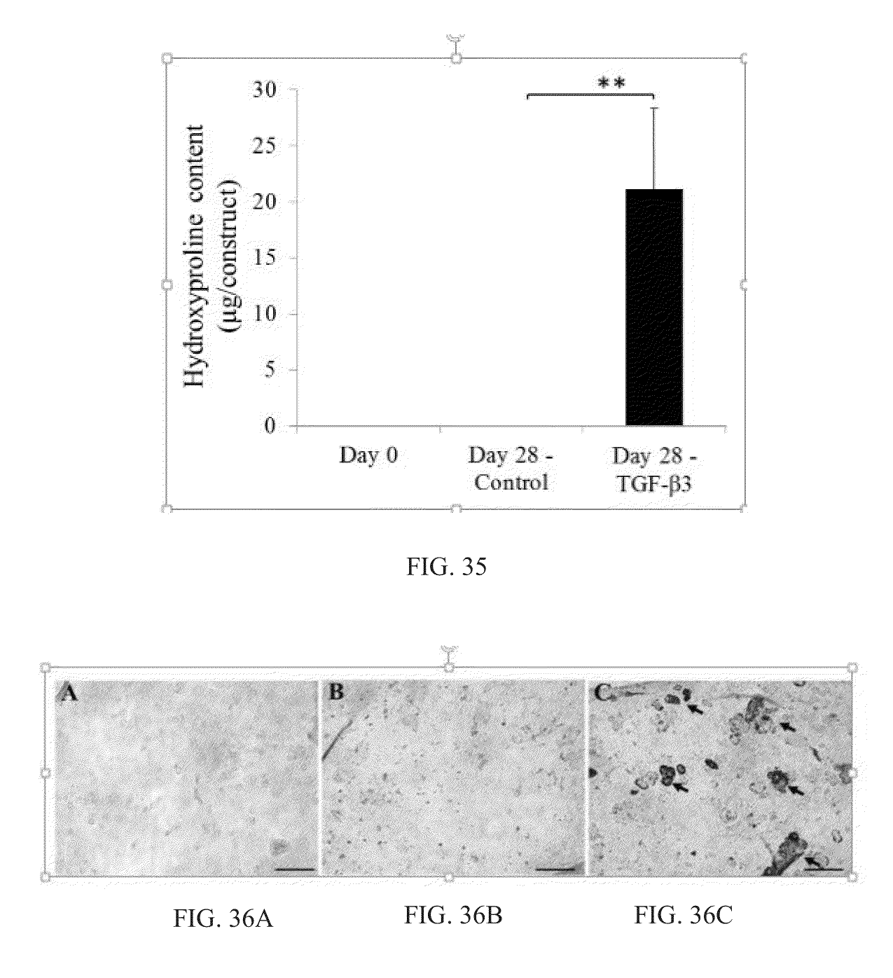

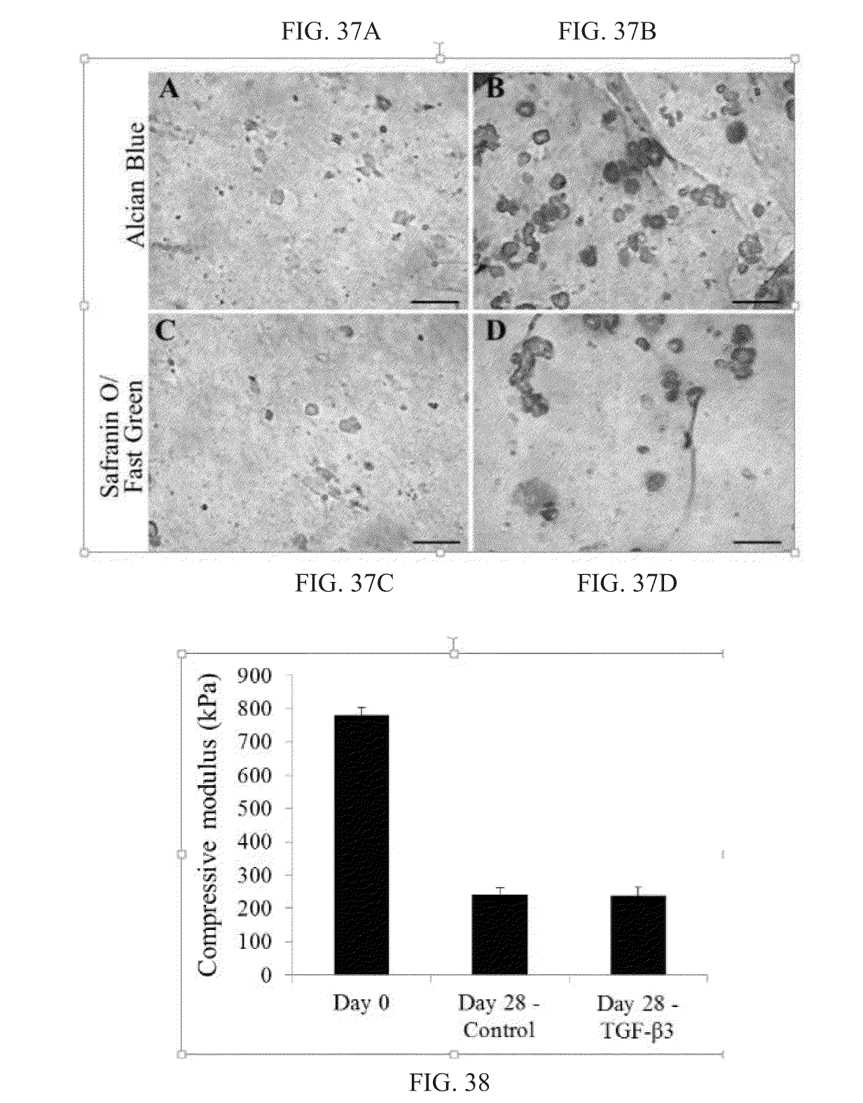

The chondrogenic activity of the cells within the constructs cultured in the chondrogenic medium was next assessed based on hydroxyproline assay to estimate the content of newly synthesized collagen. The TGF-β3 group showed a hydroxyproline level of 21.16±7.15 μg/construct versus undetectable level in the control group (FIG. 35). In addition, IHC revealed positive staining for collagen type II, a major ECM component in cartilage, only in the TGF-β3 group (FIGS. 36A-36C). Glycosaminoglycan (GAG) content was also estimated by histological staining with Alcian Blue and Safranin O, respectively. Weak staining was seen in the control group after 28 days culture (FIGS. 37A and 37C), while dense, strong staining was seen in in the TGF-β3 group (FIGS. 37B and 37D). Taken together, these results clearly demonstrate that with TGF-β3 induction, robust chondrogenic differentiation of the hASCs took place within the PSL-fabricated constructs.

Lastly, the compressive moduli for the constructs containing the mPDLLA-PEG polymer were measured. At day 0, the compressive modulus was 780±23 kPa, which fell to 240±20 kPa in the control group and 238±25 kPa in the TGF-β3 group by day 28 (FIG. 38), principally due to the degradation of the scaffold material.

### Discussion

Live cell-scaffold fabrication is an effective route towards the construction of customized grafts for tissue regeneration in vivo. Described in this example are the application of a VL-PSL protocol with a biodegradable polymer mPDLLA-PEG/HA and utilizing hASC cells to produce constructs with cartilage-like properties and high compressive moduli. Following fabrication of the scaffold, cells were uniformly distributed within the construct and cell viability was high. Importantly, chondrogenesis was observed in this dense scaffold in the group treated with TGF-β3. Real time RT-PCR showed high levels of aggrecan, collagen type II, and Sox 9 expression, characteristic of a robust chondrocytic phenotype, while Alcian Blue and Safranin O staining confirmed the presence of GAGs. Collagen production was high, consistent with the immunodetectable level of collagen type II in the TGF-β3 group. Taken together, these findings clearly indicate that the PSL-fabricated mPDLLA-PEG/HA scaffold supported the proliferation as well as robust TGF-β3 induced chondrogenesis of the encapsulated hASCs.

Tissue engineering has been considered a promising approach to repair degenerated articular cartilage in degenerative joint diseases, such as OA. The goal has been to construct or regenerate tissues that possess the properties of native cartilage, but currently there are many technical challenges that the described technology overcomes. For example, articular cartilage is an avascular tissue, which limits the ability of cells of surrounding tissues to infiltrate the defect site following implantation of the engineered tissue implant. Thus, a uniform cell distribution throughout the scaffold that makes up the construct is needed so that tissue repair can occur within the entire scaffold and not be confined to the superficial zones. Another challenge is the mechanical environment within the articular joint environment, namely the natural high loads that any scaffold or neo-tissue must withstand. The scaffold thus needs to be inherently mechanically stiff immediately following grafting. A third challenge is that articular cartilage defects, by trauma-induced loss or chronic osteoarthritic lesions, are irregular in shape, which makes moldable cell-seeded materials desirable. No currently available hydrogel materials have completely met these requirements.

As described elsewhere herein, live cells can be uniformly distributed within scaffolds with designed architectures utilizing a visible light-based PSL method for live cell-scaffold fabrication. Such methods present progress towards the challenges noted in the previous paragraph. However, the PEG material sometimes used, and other similar materials, is non-biodegradable and does not allow for conversion to native cartilage tissue. Given the limitations of the PEG methacrylate hydrogel, we have developed biomaterials that possesses the ability to be applied in VL-PSL (biocompatibility and water-solubility) as well as exhibits both higher mechanical strength and biodegradability. For example, PDLLA-PEG can provide these properties. In addition, high MW HA (>70 0kd), or similar materials, can be used to replace Percoll to maintain hASCs in suspension during fabrication, which not only eliminates the presence of Percoll but also provides native cell binding ligands. The results in FIG. 33B clearly demonstrate that cells remain sufficiently suspended uniformly throughout the scaffold during the 30-min fabrication time.

The scaffolds constructed using mPDLLA-PEG/HA rapidly degrade in aqueous solution (FIG. 32). For example, in one experiment, half of a PDLLA-PEG (4:1, w/w) scaffold was degraded in 5 days. The relative slow degradation rate of such a scaffold may be due to lower PDLLA:PEG ratio and inclusion of photocrosslinkable HA. After 12 weeks culture in HBSS, the scaffolds still maintain the original architecture.

Even with a slower degradation rate, the biodegradable scaffolds can lose 75% of strength in 4 weeks (FIG. 32). However, they are still stronger than hydrogels such as agarose, alginate and gelatin. This result indicates good mechanical properties of mPDLLA-PEG/HA. The addition of TGF-β3 maintains cell survival (77%). In addition, together with the enhanced levels of gene expression of Sox 9, aggrecan, and collagen type II as well as the presence of collage type II protein, GAGs, and proteoglycan, TGF-β3 is able to diffuse into the scaffolds and act to stimulate the encapsulated cells.

To optimize deposition of ECM, a higher initial cell loading density can be used and/or further optimized chondroinductive conditions can be employed. For example, a higher cell density such as 60×10⁶ cells/ml can result in considerably higher ECM content than low cell density culture and can improve the mechanical properties of whole construct. Also, the addition of BMP-6 can dramatically enhance the matrix production by hASCs.

In summary, this example describes new combinations of biodegradable water-soluble polymers compatible with the VL-PSL fabrication process that has the ability to accommodate seeding as well as chondrogenic differentiation of hASCs and possesses high compressive modulus and/or other mechanical properties.

Using the degradable mPDLLA-PEG/HA matrix, VL-PSL can be successfully applied to fabricate scaffolds based on CAD models as the template with high fidelity. hASCs can be introduced into the scaffolds during the fabrication and maintained high viability. TGF-β3-containing chondrogenic medium can enhance hASC survival and can also effectively induce hASCs chondrogenesis, as indicated by increased chondrogenic gene expression and cartilage ECM deposition. Live cell-based PSL-fabricated scaffolds described in this example can be used in the development of customized repair of cartilage in degenerative joint diseases, such as OA.

### EXAMPLE 9

### Combinations of Different Polymeric Materials

This example describes the use of various combinations of different polymers in tissue fabrications and how the polymer combinations benefit tissue formation. In an exemplary method, different amounts of hyaluronic acid were introduced into gelatin and cartilage formation was assessed after loading human bone marrow stem cells (hBMSCs).

Four exemplary combination groups were established: (1) 10% Gelatin, (2) 9.5% Gelatin +0.5% HA, (3) 9% Gelatin +1% HA, and (4) 8.5% Gelatin +1.5% HA. In each combination, 20 million/ml hBMSCs were incorporated. The gelation was conducted using a similar procedure to that described above in Example 3. After 9 weeks culture in TGF-β3 containing chondrogenic medium, the samples were collected; cell viability was assessed by Live/Dead staining, and chondrogenesis was assessed by real time PCR, GAG assay and histological staining.

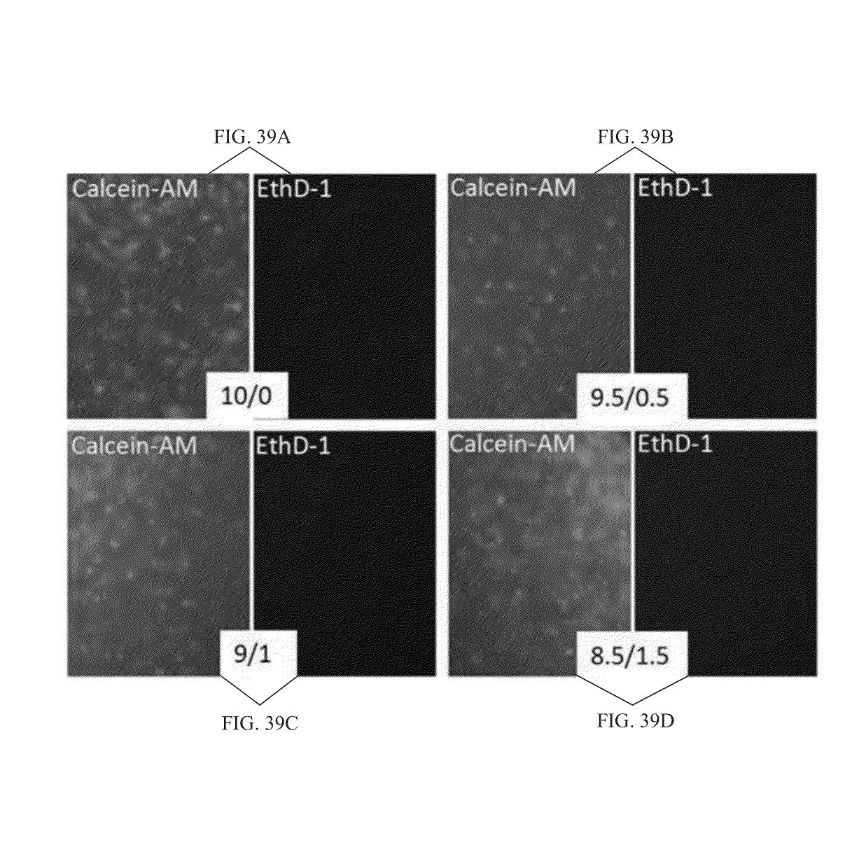

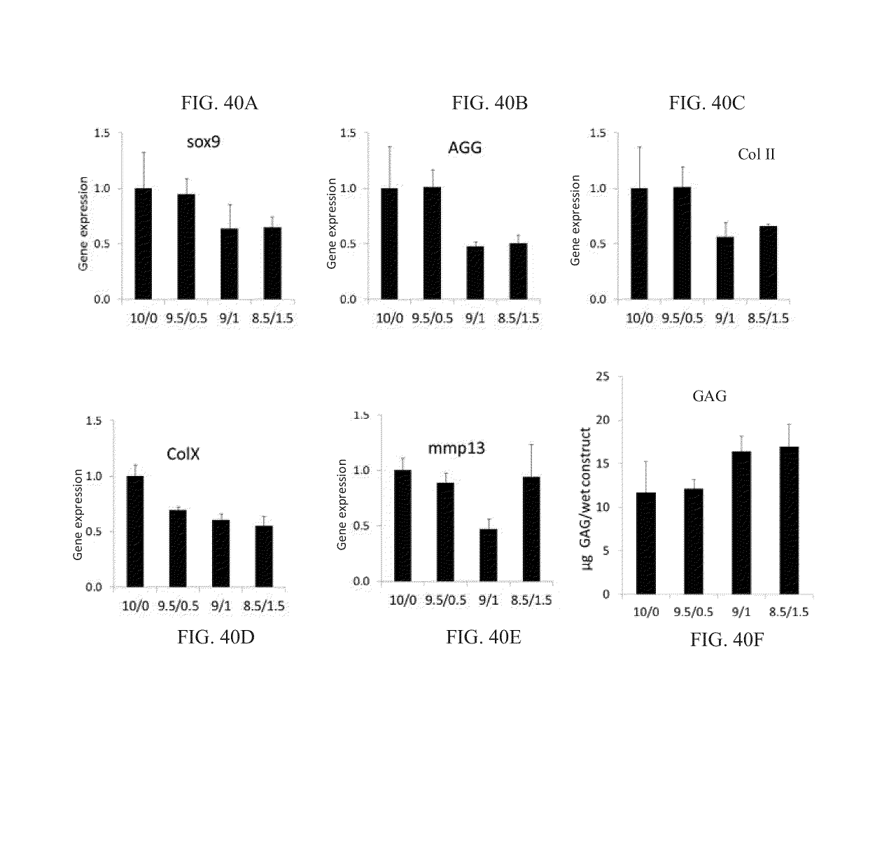

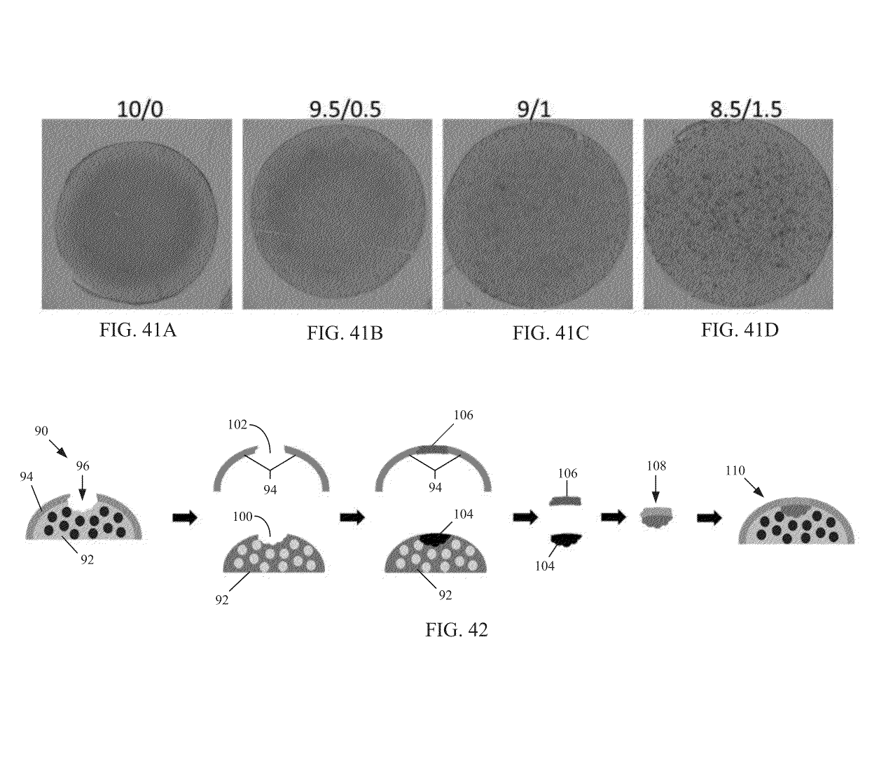

As shown in FIGS. 39A-39D, most of cells were viable in all groups. With increasing HA, the expression of chondrogenic genes decreased. However, expression of the hypertrophic genes collagen type X and MMP 13 was also down regulated (FIGS. 40A-40E), indicating the enhancement of neocartilage quality. Total glycosaminoglycan (GAG) deposited within scaffolds was then quantitated, which increased with the HA ratio. (FIG. 40F). Histological staining (FIGS. 41A-41D) also indicated robust chondrogenesis in all groups with different staining pattern.

In summary, all four combinations supported hBMSCs chondrogenesis and 9/1 appeared to be a desirable ratio.

### EXAMPLE 10

### Medical Imaging-Guided Fabrication of Human Osteochondral Tissues Using PSL

This example describes techniques for PSL fabrication of osteochondral scaffolds based on medical imaging, such as for focal or total articular cartilage surface repair (FIG. 42). FIG. 42 illustrates a method 90 for repairing a defect 96 in native osteochondral tissue comprising an interface between bone 92 and cartilage 94. The bone 92 can include a defect portion 100 and the cartilage 94 can include a defect portion 102. Cartilage tissue 106 can be fabricated to precisely match the cartilage defect portion 102 and bone tissue 104 can be fabricated to precisely match the bone defect portion 100. The fabricated tissues 104, 106 can be imaged and/or designed separately but can be fabricated by growing together as a single tissue 108 before being implanted in the defect 96 to form a repaired surface 110.

Exemplary fabrication methods involve the visible light-based photocrosslinking-based PSL methods described elsewhere herein. However, in some examples, hydrogels comprising a combination of different materials, including synthetic materials, (e.g., poly(D,L-lactide)-poly(ethylene glycol)-poly(D,L-lactide) (PDLLA-PEG)/hyaluronic acid (HA) hydrogels) can be used. Such combination hydrogels can be loaded with cells and TGFβ3 can be used to form the cartilage layer 106. Combination hydrogels can also be used to form the bone layer 104. For example, poly(ε-caprolactone) (PCL)/gelatin combination hydrogels infused with hydroxyapatite can be used to form the bone layer 104.

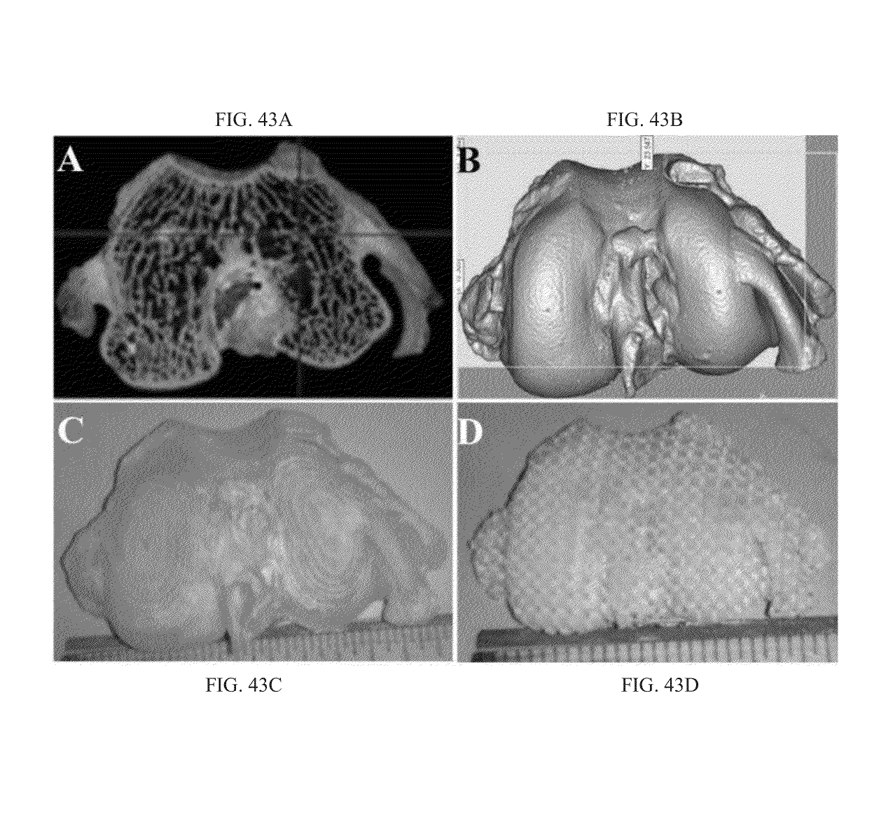

Furthermore, personalized/custom imaging of a defect site can be used instead of generic CAD models to create constructs that better match the geometry/anatomy of a specific defect site, as shown in FIGS. 43A-43D. For example, explanted joint tissue with iatrogenic osteochondral defects created after tissue harvest was incubated with Hexabrix and scanned by micro-CT. Image contrasts adjustments were made to enhance and distinguish bone versus cartilage. FIG. 43A shows an exemplary micro-CT image of osteochondral tissue with a defect site. The micro-CT imaging can be converted into 3D models (e.g., FIG. 43B) and used as the template for PSL fabrication of osteochondral tissues. FIG. 43C shows a solid scaffold fabricated from the 3D model of FIG. 43B. FIG. 43D shows a porous scaffold fabricated from the 3D model of FIG. 43B.

Using this method, custom shaped stem cell-laden osteochondral tissues were successfully created using patient-specific micro-CT imaging as the template, which had the same anatomical architecture as the original scanned native tissues. After fabrication, stem cells showed high viability (84%) and their ability to undergo osteogenesis or chondrogenesis in the bone or cartilage scaffolds, respectively, was also confirmed. Accordingly, this technology can be used for the repair of osteochondral defects.

### EXAMPLE 11

### Materials with Mechanical Properties Similar to Native Tissues

By using different materials and adjusting illumination time, we are able to make scaffolds that possess mechanical properties similar to various native tissues. For example, with 0.2% LAP, 10% gelatin (30 second illumination), 10% gelatin (5 minutes illumination), 30% PDLLA-PEG (5 minutes illumination), or 100% PCL (5 minutes illumination), fabricated scaffolds display 5.3 kPa, 35 kPa, 780 kPa or 120 MPa compressive moduli, which are similar to the stiffness of nerve, muscle, cartilage and bone respectively.

### EXAMPLE 12

### A Biocompatible, Biodegradable Synthetic Polymer that is Applicable in our Technology

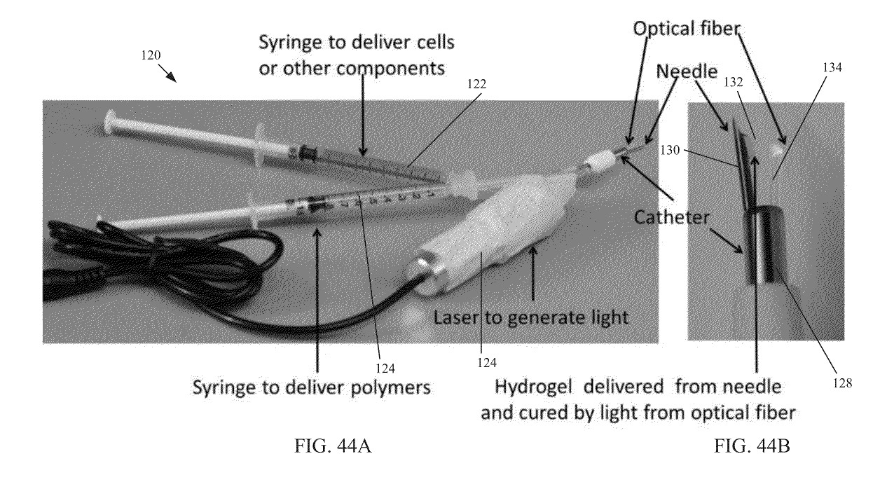

FIGS. 44A and 44B illustrate a system/device 120 that is able to simultaneously deliver polymers, cells and other liquid components via a common injection portal into a defect site, and also provide light to photocure the injected materials in the defect site. The system 120 includes a first syringe (or other delivery device) 122 that contains the polymer solution and another syringe (or other delivery device) 124 that contains live cells. In other embodiments, additional syringes or delivery devices can be included to deliver other components, such as other polymers or growth factors. The two streams from the syringes 122 and 124 meet and mix in a needle 130 and where the mixed liquid (e.g., a combination hydrogel as described above) 132 can be ejected and delivered into the defect site. The needle 130 can also be sharp to pierce tissue at or near the defect site. The system 120 can include a catheter 128 that contains both the needle 130 and an optical fiber 134, and the catheter can be operable to introduce the needle and optical fiber into the body to a defect site. The optical fiber 134 is coupled to a light source 126, such as a laser light source. When the cell-polymer mixture 132 passes through the tip of needle 130, the visible light delivered through optical fiber 134 can cure the mixture at the desired site within body in a short time period, e.g., nearly instantly. The usage of the system 120 allows for minimally invasive therapy.

For purposes of this description, certain aspects, advantages, and novel features of the embodiments of this disclosure are described herein. The disclosed methods, apparatuses, and systems should not be construed as limiting in any way. Instead, the present disclosure is directed toward all novel and nonobvious features and aspects of the various disclosed embodiments, alone and in various combinations and sub-combinations with one another. The methods, apparatuses, and systems are not limited to any specific aspect or feature or combination thereof, nor do the disclosed embodiments require that any one or more specific advantages be present or problems be solved.

Integers, characteristics, materials, and other features described in conjunction with a particular aspect, embodiment, or example of the disclosed technology are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

Although the operations of some of the disclosed methods are described in a particular, sequential order for convenient presentation, it should be understood that this manner of description encompasses rearrangement, unless a particular ordering is required by specific language. For example, operations described sequentially may in some cases be rearranged or performed concurrently. Moreover, for the sake of simplicity, the attached figures may not show the various ways in which the disclosed methods can be used in conjunction with other methods.

As used herein, the terms “a”, “an”, and “at least one” encompass one or more of the specified element. That is, if two of a particular element are present, one of these elements is also present and thus “an” element is present. The terms “a plurality of” and “plural” mean two or more of the specified element. As used herein, the term “and/or” used between the last two of a list of elements means any one or more of the listed elements. For example, the phrase “A, B, and/or C” means “A”, “B,”, “C”, “A and B”, “A and C”, “B and C”, or “A, B, and C.” As used herein, the term “coupled” generally means mechanically, chemically, or otherwise physically coupled or linked and does not exclude the presence of intermediate elements between the coupled items absent specific contrary language.

In view of the many possible embodiments to which the principles of the disclosed technology may be applied, it should be recognized that the illustrated embodiments are only examples and should not be taken as limiting the scope of the disclosure. Rather, the scope of the disclosure is at least as broad as the following claims. We therefore claim all that comes within the scope of the following claims.

1. A method of fabricating human musculoskeletal tissue, comprising:
injecting a liquid material into a musculoskeletal defect site, the liquid material comprising a biodegradable and biocompatible polymer, a photo-activated photoinitiator, and human cells capable of producing musculoskeletal tissue;
applying photoillumination to the injected liquid material within the musculoskeletal defect site to cause photocrosslinking of the polymer, such that the liquid material solidifies into a scaffold having a shape that corresponds to a shape of the musculoskeletal defect site with the human cells encapsulated within the scaffold.

2. The method of claim 1, wherein the photoinitiator is activatable by visible light, and applying photoillumination comprises applying visible light wavelength photoillumination having wavelength range from about 405 nm to about 490 nm.

3. The method of claim 1, wherein the polymer comprises natural gelatin or native collagen.

4. The method of claim 1, wherein the liquid material comprises a synthetic biodegradable polymer not native to humans.

5. The method of claim 1, wherein the polymer comprises hyaluronic acid.

6. The method of claim 1, wherein the liquid material comprises a combination polymer material in addition to the photo-activated photoinitiator and the human cells, wherein the combination polymer material comprises at least one polymer and at least a second material, and wherein the combination polymer material is biodegradable.

7. The method of claim 1, wherein the photoinitiator comprises LAP.

8. The method of claim 1, wherein the human cells comprise hBMSCs or hMSCs.

9. The method of claim 1, wherein the application of photoillumination causes the liquid material to gelate into an mGL hydrogel in the defect site.

10. The method of claim 1, wherein the liquid material comprises a soluble osteoinductive and chondroinductive biofactor.

11. The method of claim 10, wherein the osteoinductive biofactor comprises BMPs or TGF-βs.

12. The method of claim 1, wherein the liquid material comprises a viral vector.

13. The method of claim 1, further comprising methacrylating the polymer using methacrylic anhydride.

14. The method of claim 1, further comprising dissolving methacrylated gelatin in physiological saline to form a gelatin solution, and then adding LAP into the gelatin solution to form a gelatin/LAP mixture, such that the gelatin/LAP mixture is capable of producing free radicals and photocros slinking upon visible light exposure.

15. The method of claim 14, further comprising mixing human stem cells into the gelatin/LAP mixture to create an injectable liquid material.

16. The method of claim 1, wherein the musculoskeletal defect site comprises an osteochondral defect.

17. The method of claim 1, wherein the liquid material comprises predifferentiated hMSCs and the method comprises promoting chondral/osseous differentiation of the hMSCs after injection and solidification of the liquid material.

18. The method of claim 1, wherein the fabricated scaffold has physical properties of stiffness, elasticity, viscoelasticity, hardness, and/or tensile strength that are approximate that of native musculoskeletal tissue at the defect site.

19. A human cell-based engineered musculoskeletal tissue fabricated by the method of claim 1, wherein the fabricated musculoskeletal tissue mimics the physiology and function of native musculoskeletal tissue at the musculoskeletal defect site.

20. A system for fabricating human musculoskeletal tissue in situ, comprising:
an injection portion for injecting a liquid material directly into a musculoskeletal defect site, the liquid material comprising a biodegradable and biocompatible polymer, a photo-activatable photoinitiator, and human cells capable of producing musculoskeletal tissue, wherein the injection portion comprises at least a first injector configured to contain the polymer and the photo-activatable photoinitator, and at least a second injector configured to contain the human cells, wherein outputs of the first and second injectors merge and join with a needle having an outlet for ejecting the combined liquid material into the defect site; and
a photoillumination portion having a light source and a light emitter operable to deliver light to liquid material ejected from the injection portion within the musculoskeletal defect site, the light being sufficient to cause photocrosslinking of the polymer such that the light causes the liquid material to solidify into a scaffold in the defect site having a shape that corresponds to the shape of the musculoskeletal defect site with the human cells encapsulated within the scaffold.
